(19) United States
(12) Patent Application Publication
(10) Pub. No.: US 2005/0288336 A1
(43) Pub. Date: Dec. 29, 2005

(54) Cysteine protease inhibitors

(75) Inventors: Michael Graupe, Pacifica, CA (US); Agnes Lau, San Francisco, CA (US); John O. Link, San Francisco, CA (US); Yang Liu, Foster City, CA (US); Craig J. Mossman, Campbell, CA (US); John W. Patterson, Mountain View, CA (US); Sheila M. Zipfel, Mountain View, CA (US)

Correspondence Address: CELERA, AN APPLERA CORPORATION BUSINESS, 180 KIMBALL WAY, SOUTH SAN FRANCISCO, CA, 94080, US

(73) Assignee: AXYS PHARMACEUTICALS, INC., So. San Francisco, CA (US)

(21) Appl. No.: 10/514,804

(22) PCT Filed: May 14, 2003
(86) PCT No.: PCT/US03/15486

§ 371 Date: Aug. 3, 2005

(30) Foreign Application Priority Data
May 14, 2002 (US) 60380311
Oct. 30, 2002 (US) 60422337

(51) Int. Cl. A61K 31/4436; A61K 31/44; C07D 213/56

(52) U.S. Cl. 514/340; 514/357; 546/280.4; 546/336

(57) ABSTRACT

The present invention is directed to compounds that are inhibitors of cysteine protease, in particular, cathepsins B, K, L, F, and S and are therefore useful in treating diseases mediated by these proteases. The present invention is directed to pharmaceutical compositions comprising these compounds and processes for preparing them.

### BACKGROUND OF THE INVENTION

1. Field of Invention

The present invention is directed to compounds that are inhibitors of cysteine proteases, in particular Cathepsins B, K, L, F, and S and are therefore useful in treating diseases mediated by these proteases. The present invention is directed to pharmaceutical compositions comprising these compounds and processes for preparing them.

2. State of the Art

Cysteine proteases represent a class of peptidases characterized by the presence of a cysteine residue in the catalytic site of the enzyme. Cysteine proteases are associated with the normal degradation and processing of proteins. The aberrant activity of cysteine proteases, e.g., as a result of increased expression or enhanced activation, however, may have pathological consequences. In this regard, certain cysteine proteases are associated with a number of disease states, including arthritis, muscular dystrophy, inflammation, tumor invasion, glomerulonephritis, malaria, periodontal disease, metachromatic leukodystrophy and others. For example, increased cathepsin B levels and redistribution of the enzyme are found in tumors; thus, suggesting a role for the enzyme in tumor invasion and metastasis. In addition, aberrant cathepsin B activity is implicated in such disease states as rheumatoid arthritis, osteoarthritis, pneumocystis carinii, acute pancreatitis, inflammatory airway disease and bone and joint disorders.

The prominent expression of Cathepsin K in osteoclasts and osteoclast-related multinucleated cells and its high collagenolytic activity suggest that the enzyme is involved in ososteoclast-mediated bone resorption and, hence, in bone abnormalities such as occurs in osteoporosis. In addition, Cathepsin K expression in the lung and its elastinolytic activity suggest that the enzyme plays a role in pulmonary disorders as well.

Cathepsin L is implicated in normal lysosomal proteolysis as well as several disease states, including, but not limited to, metastasis of melanomas. Cathepsin S is implicated in Alzheimer's disease and certain autoimmune disorders, including, but not limited to juvenile onset diabetes, multiple sclerosis, pemphigus vulgaris, Graves' disease, myasthenia gravis, systemic lupus erythematosus, rheumatoid arthritis and Hashimoto's thyroiditis. In addition, Cathepsin S is implicated in allergic disorders, including, but not limited to asthma; and allogeneic immune reponses, including, but not limited to, rejection of organ transplants or tissue grafts.

Another cysteine protease, Cathepsin F, has been found in macrophages and is believed to be involved in antigen processing. It is believed that Cathepsin F is stimulated in lung macrophages and possibly in other antigen presenting cells and therefore could play a role in airway inflammation (see G. P. Shi et al, J. Exp. Med. 191,1177, 2000)

In view of the number of diseases wherein it is recognized that an increase in cysteine protease activity contributes to the pathology and/or symptomatology of the disease, molecules which inhibit the activity of this class of enzymes, in particular molecules which are inhibitors of Cathepsins B, K, L, F, and/or S, will therefore be useful as therapeutic agents.

### SUMMARY OF THE INVENTION

In one aspect, this invention is directed to a compound of Formula I: 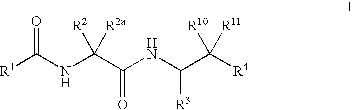
wherein:
R¹ is a group of formula: 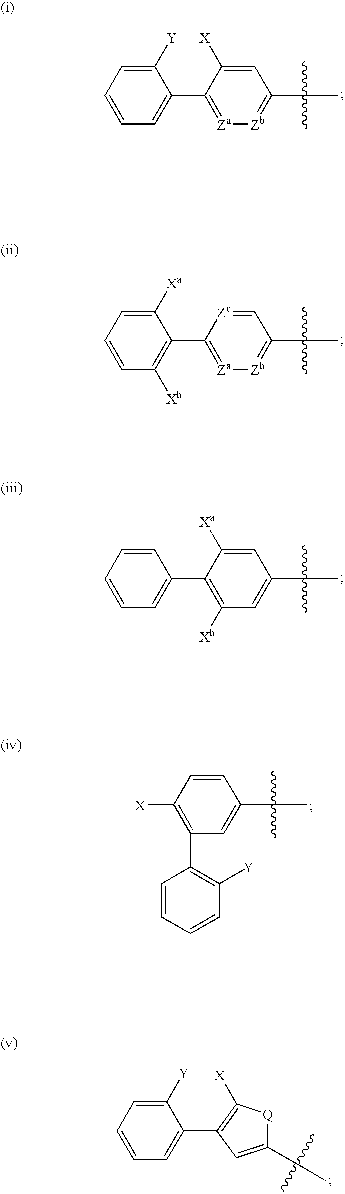 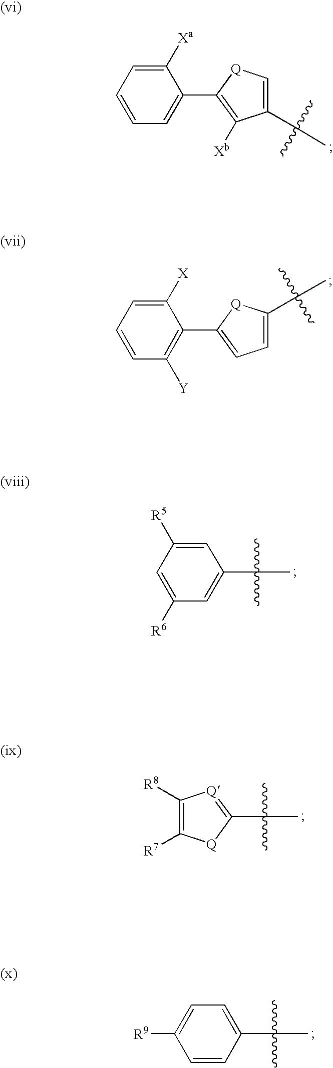 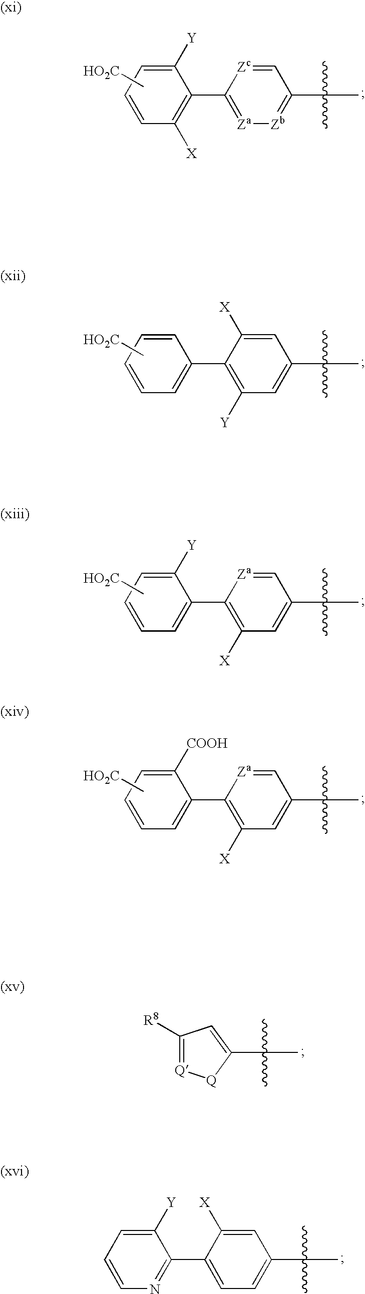
   (xix) 4-(3,5-dimethyloxazol-4-yl)phenyl;
   (xx) 4-(5-carboxy-2-methylthiophen-3-yl)phenyl;
   (xxi) 3-vinylphenyl;
   (xxii) 4-phenoxyphenyl;
   (xxiii) 4-acetylamino-3-methylphenyl; or
   (xxiv) 4-morpholin-4-ylphenyl;
   where:
Z^{a} is —CX— or —N— and Z^{b} and Z^{c} are independently selected from —CH— and —N— provided that if an R¹ group contains Z^{a}, Z^{b}, and Z^{c} simultaneously then, when Z^{c} is —N—, then Z^{a} is —N— or —CX— and Z^{b} is —H—; and when Z^{b} is —N— then both Z^{a} and Z^{c} cannot be —N— simultaneously;
Q is —NR— where R is hydrogen or alkyl, —O—, or —S—;
Q′ is —CH— or —N—;
X and Y are independently selected from hydrogen, halo, alkyl, alkoxy, haloalkyl, or haloalkoxy provided that both X and Y are not simultaneously hydrogen;
X^{a} and X^{b} are independently selected from alkyl, halo, alkoxy, haloalkyl, or haloalkoxy;
R⁵ and R⁶ are independently selected from phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl;
R⁷ and R⁸ are independently selected from phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl; and
R⁹ is a branched alkyl chain of 4-6 carbon atoms or trifluoroalkoxy;
R² is selected from the group consisting of hydrogen, methyl, ethyl, n-propyl, 2-propyl, cyclopentyl, cyclohexyl, cycloheptyl, 2-ethylbutyl, thiazolylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), benzyl (where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino), heteroaryl(C₃₋₄)alkyl and 1-heteroaryl(C₃₋₆)-cycloalkylmethyl and furthermore wherein the alkyl chain in the above groups is optionally substituted with one to six halo;
R^{2a} is hydrogen or R^{2a} and R² together with the carbon atom to which they are attached form cyclohexyl or cycloheptyl;
R³ is ethyl, propyl, or i-butyl;
R⁴ is benzoxazol-2-yl, oxazolo[4,5-b]pyridin-2-yl, 2-pyridin-3-yl-[1,3,5]-oxadiazol-5-yl, 2-pyridin-4-yl-[1,3,4]-oxadiazol-5-yl, 2-ethyl-[1,3,4]-oxadiazol-5-yl, 2-phenyl-[1,3,4]oxadiazol-5-yl, pyrazin-2-yl, pyrimidin-2-yl, pyridazin-3-yl, 3-phenyl-[1,2,4]-oxadiazol-5-yl, or 3-ethyl-[1,2,4]-oxadiazol-5-yl;
R¹⁰ is hydrogen, hydroxy, alkoxy; and
R¹¹ is hydroxy or alkoxy; or
R¹⁰ and R¹¹ together with the carbon atom to which they are attached form (═O) or —O—(C₂-C₄)alkylene-O— wherein the alkylene chain is optionally substituted with one or two alkyl; or a pharmaceutically acceptable salt thereof.

Preferably, the compound of Formula I is represent by Formula Ia: 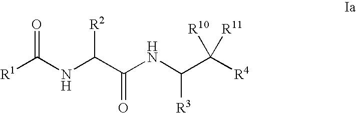
wherein:
R¹ is a group of formula: 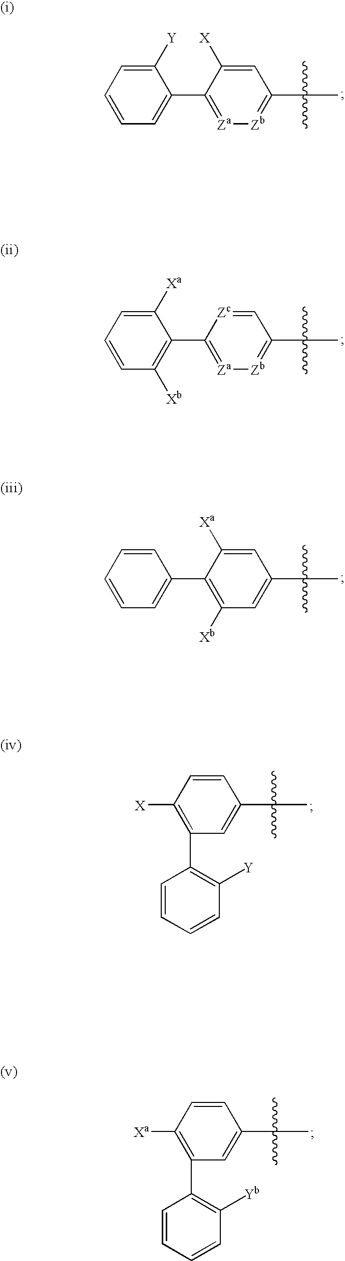 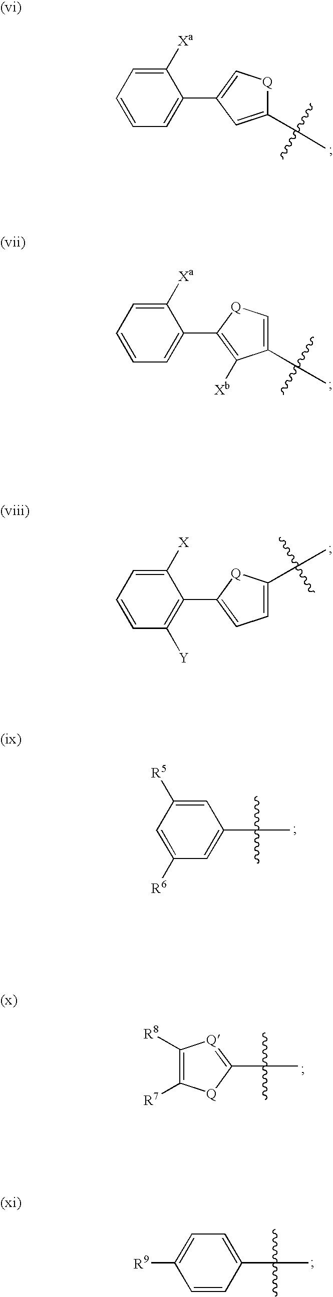
   (xii) 1-(4-aminosulfonylphenyl)-5-4-chlorophenyl)pyrazol-3-yl;
   (xiii) 1-methyl-3-trifluoro-1H-thieno-[2,3-c]-pyrazol-5-yl; 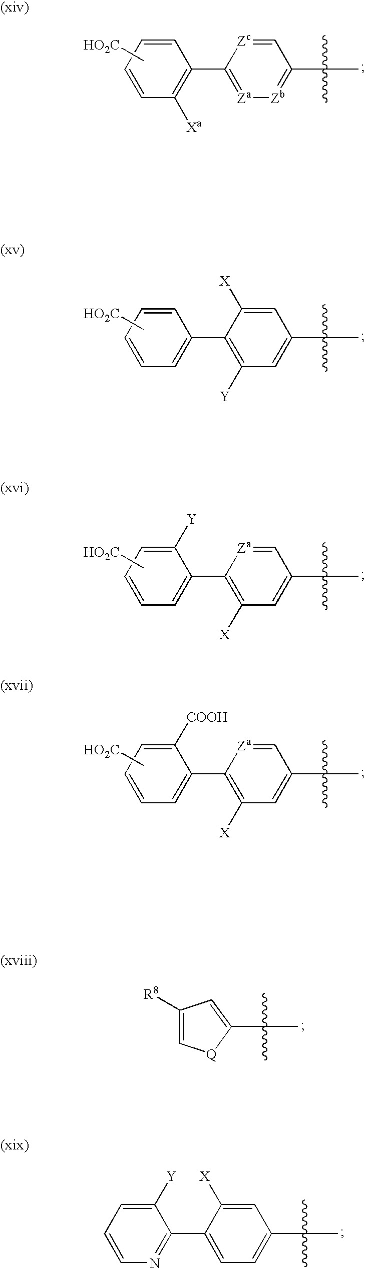
   (xx) 4-(3,5-dimethyloxazol-4-yl)phenyl; or
   (xxi) 4-(5-carboxy-2-methylthiophen-3-yl)phenyl;
   where:
Z^{a}, Z^{b}, and Z^{c} are independently selected from —H— or —N— provided that if an R¹ group contains Z^{a}, Z^{b}, and Z^{b} simultaneously then, when Z^{c} is —N—, then Z^{a} is —N— or —CH— and Z^{b} is —H—; and when Z^{b} is —N— then Z^{a} and Z_{b} are —H—; and if an R¹ group contains Zₐ and Z_{b} simultaneously, then both Zₐ and Z_{b} cannot simultaneously be —N—;
Q is —NR— where R is hydrogen or alkyl, —O—, or —S—;
Q′ is —CH— or —N—;
X and Y are independently selected from hydrogen, halo, alkyl, alkoxy, haloalkyl, or haloalkoxy provided that both X and Y are not simultaneously hydrogen;
X^{a} and X^{b} are independently selected from alkyl, halo, alkoxy, haloalkyl, or haloalkoxy,
R⁵ and R⁶ are independently selected from phenyl, 2-alkoxyphenyl, 3-alkoxyphenyl, 2-halophenyl, 2-alkylphenyl, 2-haloalkoxyphenyl, furan-2-yl, thiophen-3-yl, or pyridin-4-yl;
R⁷ and R⁸ are independently selected from phenyl, 2-alkoxyphenyl, 3-alkoxyphenyl, 2-halophenyl, 2-alkylphenyl, 2-haloalkoxyphenyl, furan-2-yl, thiophen-3-yl, or pyridin-4-yl; and
R⁹ is a branched alkyl chain of 4-6 carbon atoms or trifluoroalkoxy;
R² is selected from the group consisting of cyclopentyl, cyclohexyl, cycloheptyl, 2-ethylbutyl, thiazolylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy, and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino;
R³ is ethyl, propyl, or n-butyl;
R⁴ is benzoxazol-2-yl, oxazolo[4,5-b]pyridin-2-yl, 2-pyridin-3-yl-[1,3,5]-oxadiazol-5-yl, 2-pyridin-4-yl-[1,3,4]-oxadiazol-5-yl, 2-ethyl-[1,3,4]-oxadiazol-5-yl, 2-phenyl-[1,3,4]-oxadiazol-5-yl, pyrazin-2-yl, pyrimidin-2-yl, pyridazin-3-yl, 3-phenyl-[1,2,4]-oxadiazol-5-yl, or 3-ethyl-[1,2,4]-oxadiazol-5-yl;
R¹⁰ is hydrogen, hydroxy, alkoxy; and
R¹¹ is hydroxy or alkoxy; or
R¹⁰ and R¹¹ together with the carbon atom to which they are attached form (═O) or —O—(C₂-C₄)alkylene-O— wherein the alkylene chain is optionally substituted with one or two alkyl; or a pharmaceutically acceptable salt thereof.

More preferably, a compound of Formula Ia wherein R¹ is a group of formula: 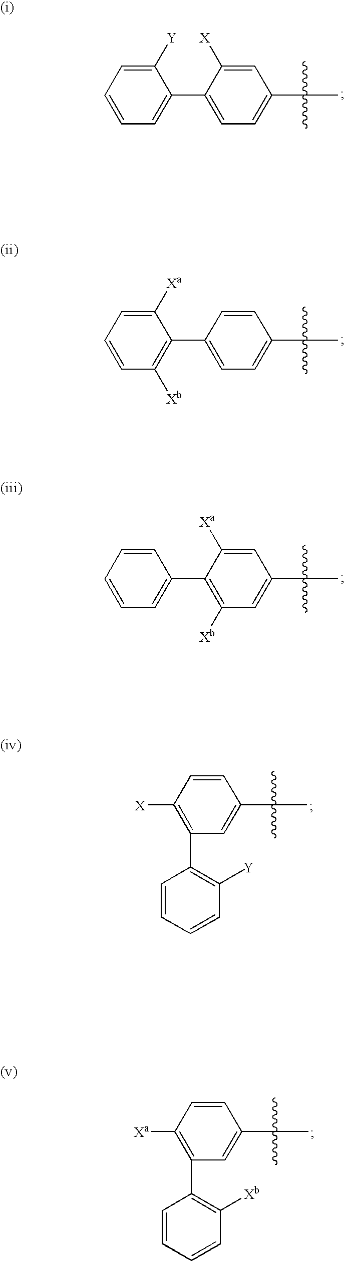 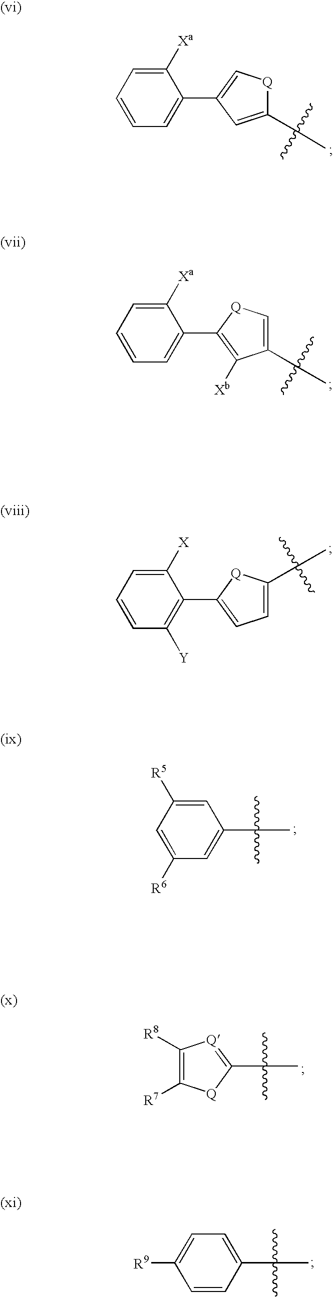
(xii) 1-(4-aminosulfonylphenyl)-5-(4-chlorophenyl)pyrazol-3-yl; or
(xiii) 1-methyl-3-trifluoro-1H-thieno-[2,3-c]-pyrazol-4-yl;
where:
Q is —NR— where R is hydrogen or alkyl, —O—, or —S—;
Q′ is —CH— or —N—;
X and Y are independently selected from hydrogen, halo, alkyl, alkoxy, or haloalkoxy provided that both X and Y are not simultaneously hydrogen;
X^{a} and X^{b} are independently selected from alkyl, halo, alkoxy, or haloalkoxy; and
R² is selected from the group consisting of cyclopentyl, cyclohexyl, 2-methylpropyl, 2,4,4-trimethylpentyl, 4-methylindol-3-ylmethyl, 2-napth-1-ylpropyl, benzyloxymethyl, 2-phenylprop-2-enyl, 2-phenyl-2-methylpropyl, 2-phenylpropyl, 2-phenylbutyl wherein the phenyl group in benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, or alkoxy, and benzyl where the phenyl ring is substituted with two halo groups;
R³ is ethyl, propyl, or n-butyl;
R⁴ is benzoxazol-2-yl, oxazolo-[4,5-b]-pyridin-2-yl, 2-pyridin-3-yl-[1,3,5]-25 oxadiazol-5-yl, 2-pyridin-4-yl-[1,3,4]-oxadiazol-5-yl, 2-ethyl-[1,3,4]-oxadiazol-5-yl, 2-phenyl-[1,3,4]-oxadiazol-5-yl, imidazol-2-yl, pyridazin-3-yl, 3-phenyl-[1,2,4]-oxadiazol-5 yl, or 3-ethyl-[1,2,4]-oxadiazol-5-yl;
R¹⁰ and R¹¹ together with the carbon atom to which they are attached form (═O); and
other groups are as defined in Formula Ia above.

In a second aspect, this invention is directed to a pharmaceutical composition comprising a compound of Formula I or Ia or a pharmaceutical acceptable salt thereof and a pharmaceutically acceptable excipient.

In a third aspect, this invention is directed to a method of treating a disease in a patient mediated by cathepsins B, K, L, F, and/or S which method comprises administering to said patient a pharmaceutical composition comprising a compound of Formula I or Ia or a pharmaceutical acceptable salt thereof and a pharmaceutically acceptable excipient. Preferably the disease is Alzheimer's disease, respiratory disease such as asthma, osteoporosis, atherosclerosis, restenosis, and autoimmune diseases such as rheumatoid arthritis, systemic lupus erythematosus, Crohn's disease, ulcerative colitis, multiple sclerosis, Guillain-Barre Syndrome, psoriasis, Grave's disease, myasthenia gravis, scleroderma, glomrulonenephritis, dermatitis, endometriosis or insulin dependent diabetes mellitus.

In a fourth aspect, this invention is directed to an intermediate of formula II: 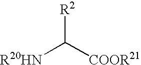
wherein:
R² is selected from the group consisting of cyclopentyl, cyclohexyl, cycloheptyl, 2-ethylbutyl, thiazolylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), benzyl (where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino), heteroaryl(C₃₋₆)alkyl and 1-heteroaryl(C₃₋₆)cycloalkylmethyl and furthermore wherein the alkyl chain in the above groups is optionally substituted with one to six halo. Preferably, R² is selected from the group consisting of cycloheptyl, 2-ethylbutyl, thiazolylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy, and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, haloalkoxy, alkylthio, alkylsulfinyl, alkylsulfonyl, cyano, amino, alkylamino, or dialkylamino;
R²⁰ is an amino-protecting group or hydrogen; preferably tert-butoxycarbonyl or benzyloxycarbonyl; and
R²¹ is a carboxy-protecting group or hydrogen.

### DETAILED DESCRIPTION OF THE INVENTION

Definitions:

Unless otherwise stated, the following terms used in the specification and claims are defined for the purposes of this Application and have the following meanings:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“Alkylene” means a linear saturated divalent hydrocarbon radical of one to six carbon atoms unless otherwise stated, e.g., (C₂₋₄)alkylene includes, but is not limited to, groups such as ethylene, propylene, 2-propylene, and butylene.

“Alkoxy” means a radical —OR where R is alkyl as defined above, e.g., methoxy, ethoxy, propoxy, or 2-propoxy, n-, iso-, or tert-butoxy, and the like, preferably methoxy.

“Alkoxyalkyloxy” means a radical —O-(alkylene)OR where R is alkyl as defined above, e.g., methoxymethyloxy, ethoxymethyloxy, 2-methoxyethyloxy, or 2-propoxyethyloxy, and the like.

“Alkoxyalkylthio” means a radical —S-(alkylene)OR where R is alkyl as defined above, e.g., methoxymethylthio, ethoxymethylthio, 2-methoxyethylthio, or 2-propoxyethylthio, and the like.

“Aminoalkyloxy” means a radical —O-(alkylene)NRR′ where R and R′ are independently hydrogen or alkyl as defined above, e.g., methylaininoethyloxy, dimethylaminoethyloxy, and the like.

“Aminoalkylthio” means a radical —S-(alkylene)NRR′ where R and R′ are independently hydrogen or alkyl as defined above, e.g., methylaminoethylthio, dimethylaminoethylthio, and the like.

“Alkylthio” means a radical —SR where R is alkyl as defined above, e.g., methylthio, ethylthio, and the like.

“Alkylsulfinyl” means a radical —S(O)R where R is alkyl as defined above, e.g., methylsulfinyl, ethylsulfinyl, and the like.

“Alkylsulfonyl” means a radical —S(O)₂R where R is alkyl as defined above, e.g., methylsulfonyl, ethylsulfonyl, and the like.

“Alkylamino” means a radical —NHR where R is alkyl as defined above, e.g., methylamino, ethylamino, and the like.

“Cycloalkyl” means a cyclic monovalent saturated monovalent hydrocarbon radical of three to six carbon atoms unless otherwise indicated e.g., cyclopropyl, cyclobutyl, and the like, preferably cyclopropyl.

“Dialkylamino” means a radical —NRR′ where R and R′ are independently alkyl as defined above, e.g., dimethylamino, methylethylamino, and the like.

“Halo” means fluoro, chloro, bromo, and iodo, preferably fluoro or chloro.

“Haloalkyl” means alkyl substituted with one or more halogen atoms, preferably one to three halogen atoms, preferably fluorine or chlorine, including those substituted with different halogens, e.g., —CH₂Cl, —CF₃, —CHF₂, and the like, preferably trifluoromethyl.

“Haloalkoxy” means a radical —OR where R is haloalkyl as defined above, e.g., trifluoromethoxy, 2,2,2-trifluoroethoxy, difluoromethoxy, and the like, preferably trifluoromethoxy.

“Heteroaryl” means a monovalent monocyclic or bicyclic aromatic radical of 5 to 10 ring atoms containing one or more, preferably one or two ring heteroatoms selected from N, O, or S, the remaining ring atoms being carbon. The heteroaryl ring is optionally substituted with one or more substituents, preferably one or two substituents, independently selected from alkyl, haloalkyl, alkoxy, alkylthio, halo, nitro, cyano, amino, alkyl or dialkylamino, hydroxy, carboxy, or —COOR where R is alkyl as define above. More specifically the term heteroaryl includes, but is not limited to, pyridyl, pyrrolyl, imidazolyl, thienyl, furanyl, indolyl, quinolyl, pyrazine, pyrimidine, pyradizine, oxazole, isooxazolyl, benzoxazole, quinoline, isoquinoline, benzopyranyl, thiazolyl, and the like.

“Heteroaryl(C₃₋₆)alkyl” means an alkylene chain of three to six carbon atoms carrying a heteroaryl group as defined above.

“1-Heteroaryl(C₃₋₆)cycloalkylmethyl” means a radial of the formula: 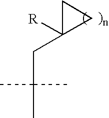
where R is a heteroaryl group as defined above and n is 1, 2, 3 or 4. Representative examples include, but are not limited to, 1-pyridin-2-ylcyclopropylmethyl, 1-pyridin-2-ylcyclobutylmethyl, and the like.

The present invention also includes the prodrugs of compounds of Formula I. The term prodrug is intended to represent covalently bonded carriers, which are capable of releasing the active ingredient of Formula I when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of Formula I are also within the scope of this invention.

The present invention also includes N-oxide derivatives and protected derivatives of compounds of Formula I. For example, when compounds of Formula I contain an oxidizable nitrogen atom, the nitrogen atom can be converted to an N-oxide by methods well known in the art or in vivo. For example, the nitrogen atom in a pyridyl group in a compound of Formula I can be oxidized to give a corresponding pyridyl-N-oxide compound of Formula I.

A “pharmaceutically acceptable salt” of a compound means a salt that is pharmaceutically acceptable and that possesses the desired pharmacological activity of the parent compound. Such salts include:
acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The compounds of the present invention may have asymmetric centers. Compounds of the present invention containing an asymmetrically substituted atom may be isolated in optically active or racemic forms. It is well known in the art how to prepare optically active forms, such as by resolution of materials. All enantiomeric, diastereomeric, and racemic forms are within the scope of this invention, unless the specific stereochemistry or isomeric form is specifically indicated.

Additionally, as used herein the terms alkyl includes all the possible isomeric forms of said alkyl group albeit only a few examples are set forth.

“Optional” or “optionally” means that the subsequently described event or circumstance may but need not occur, and that the description includes instances where the event or circumstance occurs and instances in which it does not. For example, the term “phenyl group optionally with an alkyl group” means that the alkyl may but need not be present, and the description includes situations where the phenyl group is substituted with an alkyl group and situations where the phenyl group is not substituted with the alkyl group.

A “pharmaceutically acceptable carrier or excipient” means a carrier or an excipient that is useful in preparing a pharmaceutical composition that is generally safe, nontoxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Treating” or “treatment” of a disease includes:
(1) preventing the disease, i.e. causing the clinical symptoms of the disease not to develop in a mammal that may be exposed to or predisposed to the disease but does not yet experience or display symptoms of the disease;
(2) inhibiting the disease, i.e., arresting or reducing the development of the disease or its clinical symptoms; or
(3) relieving the disease, i.e., causing regression of the disease or its clinical symptoms.

A “therapeutically effective amount” means the amount of a compound of Formula I that, when administered to a mammal for treating a disease, is sufficient to effect such treatment for the disease. The “therapeutically effective amount” will vary depending on the compound, the disease and its severity and the age, weight, etc., of the mammal to be treated.

### Preferred Embodiments

While the broadest definition of this invention is set forth in the Summary of the Invention, certain compounds of Formula I are preferred. For example:

1. A preferred group of compounds of Formula I is represented by Formula Ib: 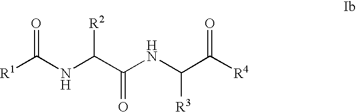
is that wherein:
R¹ is a group of formula: 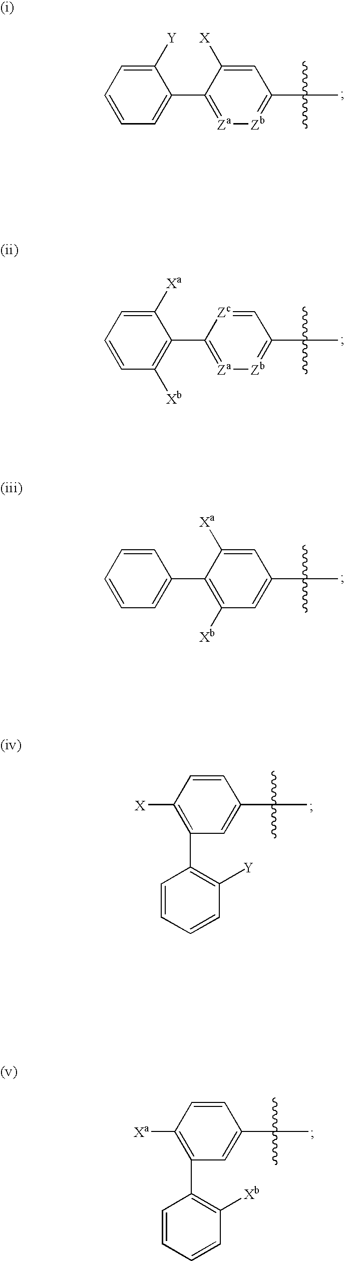 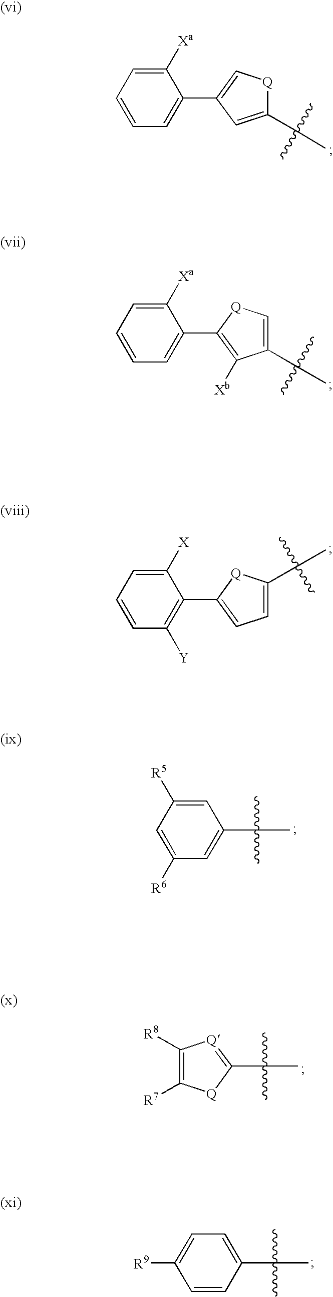
   (xii) 1-(4-aminosulfonylphenyl)-5-(4-chlorophenyl)pyrazol-3-yl; or
   (xiii) 1-methyl-3-trifluoro-1H-thieno-[2,3-c]-pyrazol-4-yl;
   wherein:
Z^{a}, Z^{b} and Z^{c} are —CH—;
Q is —NR— where R is hydrogen or alkyl, —O—, or —S—;
Q′ is —CH— or —N—;
   X and Y are independently selected from hydrogen, chloro, methyl, methoxy, trifluoromethyl, or trifluoromethoxy;
X^{a}, and X^{b} are independently selected from methyl, chloro, fluoro, methoxy, trifluoromethyl, or trifluoromethoxy;
R⁵ and R⁶ are independently selected from phenyl, 2-alkoxyphenyl, 3-alkoxyphenyl, 2-halophenyl, 2-alkylphenyl, 2-haloalkoxyphenyl, furan-2-yl, thiophen-3-yl, or pyridin-4-yl;
R⁷ and R⁸ are independently selected from phenyl, 2-alkoxyphenyl, 3-alkoxyphenyl, 2-halophenyl, 2-alkylphenyl, 2-haloalkoxyphenyl, furan-2-yl, thiophen-3-yl, or pyridin-4-yl;
R² is selected from the group consisting of cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy) and benzyl (where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino, preferably the phenyl ring in the benzyl group is substituted at the 2 and 6 positions with groups independently selected from methyl, chloro, fluoro, trifluoromethyl, methoxy, trifluoromethoxy, or difluoromethoxy and at the 4 position with hydrogen, methyl, ethyl, propyl, chloro, fluoro, trifluoromethyl, methoxy, 2-methoxyethyloxy, 2-dimethylaminoethyloxy, trifluoromethoxy, difluoromethoxy, methylthio, methylsulfinyl, methylsulfonyl, cyano, amino, methylamino or dimethylamino). R² is preferably selected from the group consisting of cyclohexyl, cycloheptyl, thiazol-2-ylmethyl, 2-ethylbutyl, pyrazol-1-ylmethyl, 2-methylpropyl, 2,4,4-trimethylpentyl, 2-napth-1-ylpropyl, 2-phenylprop-2-enyl, 2-phenyl-2-methylpropyl, 2-phenylpropyl, 2-(2-methoxyphenyl)propyl, 4-methylindol-3-ylmethyl, 2-(2,5-dimethylphenyl)propyl, benzyloxymethyl, 2-(2,4-dimethylphenyl)propyl, 2-(2,4-dichlorophenyl)-propyl, 2,6-difluorobenzyl, 2,5-difluorobenzyl, and 2,3-difluorobenzyl;
R⁴ is benzoxazol-2-yl, oxazolo[4,5-b]pyridin-2-yl, 2-pyridin-3-yl-[1,3,5]-oxadiazol-5-yl, 2-pyridin-4-yl-[1,3,4]-oxadiazol-5-yl, 2-ethyl-[1,3,4]-oxadiazol-5-yl, 2-phenyl-[1,3,4]-oxadiazol-5-yl, pyrazin-2-yl, pyrimidin-2-yl, pyridazin-3-yl, 3-phenyl-[1,2,4]-oxadiazol-5-yl, or 3-ethyl-[1,2,4]-oxadiazol-5-yl and R⁹ is as defined in the Summary of the Invention.

With the above group, a more preferred group of compounds is that wherein:
X and Y are independently selected from hydrogen, chloro, methyl, methoxy, or trifluoromethoxy, preferably hydrogen, chloro, methyl, or methoxy;
X^{a}, and X^{b} are independently selected from methyl, chloro, fluoro, methoxy, or trifluoromethoxy, preferably chloro, methyl, or methoxy;
R² is selected from the group consisting of 2-methylpropyl, 2,4,4-trimethylpentyl, 2-napth-1-ylpropyl, 2-phenylprop-2-enyl, 2-phenyl-2-methylpropyl, 2-phenylpropyl, 2-(2-methoxy-phenyl)propyl, 4-methylindol-3-ylmethyl, 2-2,5-dimethylphenyl)propyl, benzyloxymethyl, 2-(2,4-dimethylphenyl)propyl, 2-(2,4-dichlorophenyl)propyl, 2,6-difluorobenzyl, 2,5-difluorobenzyl, and 2,3-difluorobenzyl; preferably 2,6-difluorobenzyl or 2S-phenylpropyl and the stereochemistry at the carbon atom to which R² is attached is (S) when the Prelog rule places the order of the substituent 1) N, 2) —COOH, 3) R² and 4) H; and (R) when the Prelog rule places the order of the substituent 1) N, 2) R², 3) —COOH and 4) H;
R⁴ is benzoxazol-2-yl, oxazolo[4,5-b]pyridin-2-yl, 2-pyridin-3-yl-[1,3,5]-oxadiazol-5-yl, 2-pyridin-4-yl-[1,3,4]-oxadiazol-5-yl, 2-ethyl-[1,3,4]-oxadiazol-5-yl, 2-phenyl-[1,3,4]-oxadiazol-5-yl, pyridazin-3-yl, 3-phenyl-[1,2,4]-oxadiazol-5-yl, or 3-ethyl-[1,2,4]-oxadiazol-5-yl; and
the stereochemistry at the carbon atom to which R³ is attached is (S).

Within the above preferred and more preferred groups, an even more preferred group of compounds is that wherein R¹ is 2′-chlorobiphen-4-yl, 2′,3-dichlorobiphenyl-4-yl, 2′,6′-dichlorobiphen-4-yl, 2′,6′-dimethylbiphen-4-yl, 2′-methylbiphen-4-yl, 2′-fluorobiphen-4-yl; 4-trifluoromethoxyphenyl, 4-(2-butyl)phenyl, 3,5-diphenylphenyl, 2,3-diphenylthiophen-5-yl, 2-(2-methylphenyl)furan-5-yl, 2-(2-methoxyphenyl)furan-5-yl, 3-methoxy-2-(2-methylphenyl)thiophen-4-yl, 3-methoxy-2-(2-methoxyphenyl)thiophen-4-yl, 2,3-di(2-methoxyphenyl)thiophen-5-yl, 3,5-di(2-methoxyphenyl)phenyl, 3,5-di(3-methoxyphenyl-phenyl, 3,5-di(thiophen-3-yl)phenyl, 3,5-di(pyridin-4-yl)phenyl, 2,3-di(2-methylphenyl)-thiophen-5-yl, 4-tert-butylphenyl, 2,3-di(furan-2-yl)thiophen-5-yl, 3,5-di(furan-2-yl)phenyl, 4-(2-methylphenyl)thiophen-2-yl, 4-(2-methoxyphenyl)thiophen-2-yl, 2′-chlorobiphen-3-yl, 2′-methyl-4-chlorobiphenyl-3-yl, 3,5-di(2-chlorophenyl)phenyl, 2,3-di(2-chlorophenyl)thiophen-5-yl, 1-(4-aminosulfonylphenyl)-5-(4-chlorophenyl)pyrazol-3-yl, 2-(2,6-dichlorophenyl)furan-5-yl, 2,3-diphenylthiophen-5-yl, 4,5-diphenylthiazol-2-yl, 3-trifluoromethyl-1-methylthieno[2,3-c]pyrazol-5-yl, 3-methylbiphen-4-yl, 2′-methoxybiphen-4-yl, 2′-trifluoromethylbiphen-4-yl, or 2′-methyl-3-chlorobiphen-4-yl.

More preferably, R¹ is 2′-chlorobiphen-4-yl, 2′,3-dichlorobiphenyl-4-yl, 2′,6′-dichlorobiphen-4-yl, 2′,6′-dimethylbiphen-4-yl, 2′-methylbiphen-4-yl, 2′-fluorobiphen-4-yl; 4-trifluoromethoxy-phenyl, 4-(2-butyl)phenyl, 3,5-diphenylphenyl, 2,3-diphenylthiophen-5-yl, 2-(2-methylphenyl)-furan-5-yl, 2-(2-methoxyphenyl)furan-5-yl, 3-methoxy-2-(2-methylphenyl)thiophen-4-yl, 3-methoxy-2-(2-methoxyphenyl)thiophen-4-yl, 2,3-di(2-methoxyphenyl)thiophen-5-yl, 3,5-di(2-methoxyphenyl)phenyl, 3,5-di(3-methoxyphenyl)phenyl, 3,5-di(thiophen-3-yl)phenyl, 3,5-di(pyridin-4-yl)phenyl, 2,3-di(2-methylphenyl)thiophen-5-yl, 4-tert-butylphenyl, 2,3-di(furan-2-yl)thiophen-5-yl, 3,5-di(furan-2-yl)phenyl, 4-(2-methylphenyl)thiophen-2-yl, 4-(2-methoxyphenyl)-thiophen-2-yl, 2′-chlorobiphen-3-yl, 2′-methyl-4-chlorobiphenyl-3-yl, 3,5-di(2-chlorophenyl)-phenyl, 2,3-di(2-chlorophenyl)thiophen-5-yl, 1-(4-aminosulfonylphenyl)-5-(4-chlorophenyl)-pyrazol-3-yl, 2-(2,6-dichlorophenyl)furan-5-yl, 2,3-diphenylthiophen-5-yl, 4,5-diphenylthiazol-2-yl, or 3-trifluoromethyl-1-methylthieno[2,3-c]pyrazol-5-yl. Even more preferably, R¹ is 2′-chlorobiphen-4-yl, 2′,6′-dichlorobiphen-4-yl or 2′,3-dichlorobiphen-4-yl.

Within these preferred and more preferred groups, a particularly preferred group of compounds is that wherein R³ is ethyl.

Within these preferred, more preferred groups, and particularly preferred groups, a more particularly preferred group of compounds is that wherein R⁴ is benzoxazol-2-yl.

2. Another preferred group of compounds of Formula I is represented by Formula IC: 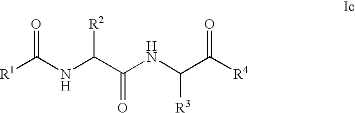
is that wherein:
R¹ is a group of formula: 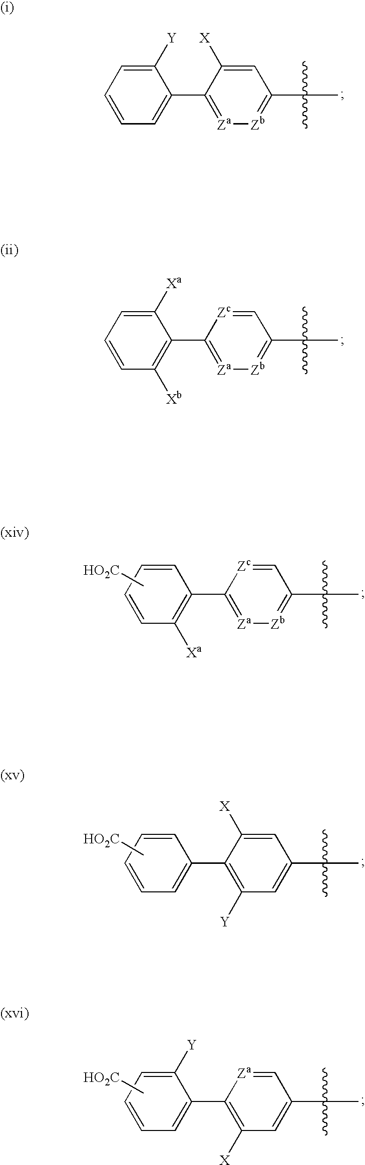 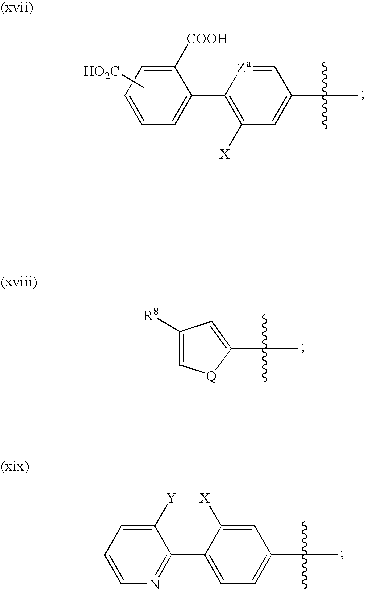
   (xx) 4-(3,5-dimethyloxazol-4-yl)phenyl; or
   (xxi) 4-(5-carboxy-2-methylthiophen-3-yl)phenyl;
   wherein:
Z^{a}, Z^{b}, and Z^{c} are independently selected from —CH— or —N— provided that when R¹ is a group of formula (i) then one of Z^{a} and Z^{b} is —N— and the other is —CH—; when R¹ is a group of formula (ii), then Z^{c} is —N—, Z^{a} is —N— or —H— and Z^{b} is —H—; or Z^{b} is —N— and Z^{a} and Z^{c} are —H—; and when R¹ is a group of formula (xiv), then when Z^{c} is —N—, then Z^{a} is —N— or —H—, and Z^{b} is —CH—; and when Z^{b} is —N—, then Z^{a} and Z^{c} are —H—;
X and Y are independently selected from hydrogen, chloro, methyl, methoxy, trifluoromethyl, or trifluoromethoxy, preferably hydrogen, chloro, methyl, methoxy, or trifluoromethoxy, more preferably hydrogen, chloro, methyl, or methoxy;
Z^{a}, and X^{b} are independently selected from methyl, chloro, fluoro, methoxy, trifluoromethyl, or trifluoromethoxy, preferably methyl, chloro, fluoro, methoxy, or trifluoromethoxy, more preferably chloro, methyl, or methoxy;
R⁸ is phenyl, 2-alkoxyphenyl, 3-alkoxyphenyl, 2-halophenyl, 2-alkylphenyl, 2-haloalkoxyphenyl, furan-2-yl, thiophen-3-yl, or pyridin-4-yl;
R² is selected from the group consisting of cyclopentyl, cyclohexyl, cycloheptyl, 2-ethylbutyl, thiazolylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy) and benzyl (where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino, preferably the phenyl ring in the benzyl group is substituted at the 2 and 6 positions with groups independently selected from methyl, chloro, fluoro, trifluoromethyl, methoxy, trifluoromethoxy, or difluoromethoxy and at the 4 position with hydrogen, methyl, ethyl, propyl, chloro, fluoro, trifluoromethyl, methoxy, 2-methoxyethyloxy, 2-dimethylaminoethyloxy, trifluoromethoxy, difluoromethoxy, methylthio, methylsulfinyl, methylsulfonyl, cyano, amino, methylamino or dimethylamino). R² is preferably selected from the group consisting of cyclohexyl, cycloheptyl, thiazol-2-ylmethyl, 2-ethylbutyl, pyrazol-1-ylmethyl, 2-methylpropyl, 2,4,4-trimethylpentyl, 2-napth-1-ylpropyl, 2-phenylprop-2-enyl, 2-phenyl-2-methylpropyl, 2-phenylpropyl, 2-(2-methoxyphenyl)propyl, 4-methylindol-3-ylmethyl, 2-(2,5-dimethylphenyl)propyl, benzyloxymethyl, 2-(2,4-dimethyl-phenyl)propyl, 2-(2,4-dichlorophenyl)propyl, 2,6-difluorobenzyl, 2,5-difluorobenzyl, and 2,3-difluorobenzyl; even more preferably 2-methylpropyl, 2,4,4-trimethylpentyl, 2-napth-1-yl-propyl, 2-phenylprop-2-enyl, 2-phenyl-2-methylpropyl, 2-phenylpropyl, 2-(2-methoxyphenyl)propyl, 4-methylindol-3-ylmethyl, 2-(2,5-dimethylphenyl)propyl, benzyloxymethyl, 2-(2,4-dimethyl-phenyl)propyl, 2-(2,4-dichlorophenyl)propyl, 2,6-difluorobenzyl, 2,5-difluorobenzyl, and 2,3-difluorobenzyl; particularly preferably R² is 2,6-difluorobenzyl or 2S-phenylpropyl and the stereochemistry at the carbon to which R² is attached is (S) when the Prelog rule places the order of the substituent 1) N, 2) COOH, 3) R² and 4) H; and (R) when the Prelog rule places the order of the substituent 1) N, 2) R², 3) COOH and 4) H;
R⁴ is benzoxazol-2-yl, oxazolo[4,5-b]pyridin-2-yl, 2-pyridin-3-yl-[1,3,5]-oxadiazol-5-yl, 2-pyridin-4-yl-[1,3,4]-oxadiazol-5-yl, 2-ethyl-[1,3,4]-oxadiazol-5-yl, 2-phenyl-[1,3,4]-oxadiazol-5-yl, pyrazin-2-yl, pyrimidin-2-yl, pyridazin-3-yl, 3-phenyl-[1,2,4]-oxadiazol-5-yl, or 3-ethyl-[1,2,4]-oxadiazol-5-yl; and
the stereochemistry at the carbon atom to which R³ is attached is (S).

Within the above preferred and more preferred groups, an even more preferred group of compounds is that wherein R¹ is 2-(2-chlorophenyl)pyridin-5-yl, 2-(2,6dichlorophenyl)pyridin-5-yl, 2-(2-trifluoromethylphenyl)pyridin-5-yl, 4-(3-methylpyridin-2-yl)phenyl, 2-(2-chlorophenyl)-3-chloropyridin-5-yl, 4′-carboxy-2′-chlorobiphen-4-yl, 4′-carboxy-2′-fluorobiphen-4-yl, 4′-carboxy-2′-methylbiphen-4-yl, 5′-carboxy-2′-methylbiphen-4-yl, 4-(3-methylpyridin-2-yl)phenyl, 5′-carboxy-2′-chlorobiphen-4-yl, 2-(4-carboxy-2-chlorophenyl)pyridin-5-yl, 2-(5-carboxy-2-chlorophenyl)pyridin-5-yl, 4-(5-carboxy-2-methylthiophen-3-yl)phenyl, or 4-(3-methoxy-phenyl)thiophen-2-yl. More preferably R¹ is 2-(2-chlorophenyl)pyridin-5-yl.

Within these preferred and more preferred groups, a particularly preferred group of compounds is that wherein R³ is ethyl.

Within these preferred, more preferred groups, and particularly preferred groups, a more particularly preferred group of compounds is that wherein R⁴ is benzoxazol-2-yl.

3. Yet another preferred group of compounds of Formula I is represented by Formula Id: 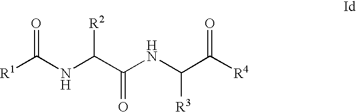
wherein:
R¹ is a group of formula: 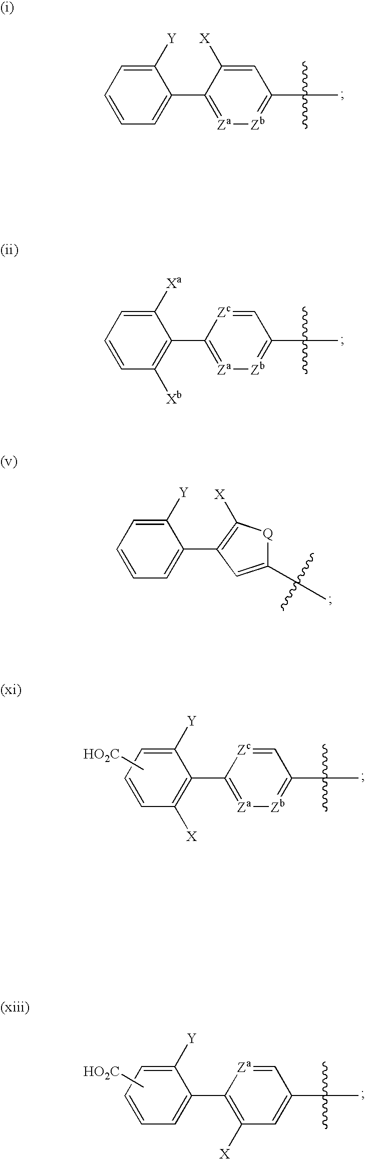 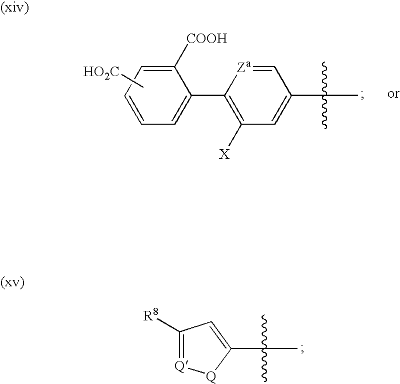
   where:
Z^{a} is —CX— or —N— and Z^{b} and Z^{c} are independently selected from —CH— and —N—, provided that if an R¹ group contains Z^{a}, Z^{b}, and Z^{c} simultaneously then, when Z^{c} is —N—, then Z^{a} is —N— or —CX— and Z^{b} is —CH—; and when Z^{b} is —N— then both Z^{a} and Z^{c} cannot be —N— simultaneously and further provided that X is not hydrogen; and
Q, Q′, X, Y, X^{a}, X^{b}, R², R³, R⁴ and R⁸ are as defined in the Summary of the Invention for Formula I.

More preferably, X and Y are independently selected from hydrogen, chloro, methyl, methoxy, trifluoromethyl, or trifluoromethoxy, preferably hydrogen, chloro, methyl, or methoxy;
X^{a}, and X^{b} are independently selected from methyl, chloro, fluoro, methoxy, trifluoromethyl, or trifluoromethoxy, preferably chloro, methyl, or methoxy; and
R² is selected from the group consisting of cyclohexyl, cycloheptyl, thiazol-2-ylmethyl, 2-ethylbutyl, pyrazol-1-ylmethyl, 2-methylpropyl, 2,4,4-trimethylpentyl, 2-napth-1-ylpropyl, 2-phenylprop-2-enyl, 2-phenyl-2-methylpropyl, 2-phenylpropyl, 2-(2-methoxyphenyl)propyl, 4-methylindol-3-ylmethyl, 2-(2,5-dimethylphenyl)propyl, benzyloxymethyl, 2-(2,4-dimethylphenyl)propyl, 2-(2,4-dichlorophenyl)-propyl, 2,6-difluorobenzyl, 2,5-difluorobenzyl, and 2,3-difluorobenzyl; preferably 2,6-difluorobenzyl or 2-phenylpropyl and the stereochemistry to which R² is attached is (S) when the Prelog rule places the order of the substituent 1) N, 2) —COOH, 3) R² and 4) H; and (R) when the Prelog rule places the order of the substituent 1) N, 2) R², 3) —COOH and 4) H; and
the stereochernistry at the carbon atom to which R³ is attached is (S).

Within the above preferred and more preferred groups, an even more preferred group of compounds is that wherein R¹ is 3-chloro-2-(2,6-dichlorophenyl)pyridin-5-yl or 3-(2-chlorophenyl)isoxazol-5-yl; R³ is ethyl, and R⁴ is benzoxazol-2-yl.

4. Yet another preferred group of compounds of Formula I is that wherein R¹⁰ is hydrogen, hydroxy, alkoxy; and R¹¹ is hydroxy or alkoxy; or R¹⁰ and R¹¹ together with the carbon atom to which they are attached form —O—C₂-C₄)alkylene-O— wherein the alkylene chain is optionally substituted with one or two alkyl.

Within this group a more preferred group of compounds is that wherein R¹, R², R³ and R⁴ are preferred groups disclosed in groups 1-3 above.

5. Yet another preferred group of compounds of Formula I is that wherein:
R¹ is 2′-Cl-biphenyl-4-yl, 2,3-diphenylthiophen-5-yl, 2-(2-Clphenyl)pyridin-5-yl, 2′,3-diCl-biphen-4-yl, 5′-carboxy-2′chlorobiphen-4-yl, 3-vinylphenyl, 4-phenoxyphenyl, 4-acetylamino-3-methyl-phenyl, 3,5-di(2-methoxyphenyl)-phenyl, 4-morpholin-4-ylphenyl, 1-methyl-3-trifluoromethyl-1H-thieno[2,3-c]pyrazol-5-yl, or 4-tert-butylphenyl;
R² is 2,6-difluorobenzyl, 2(S)-phenylpropyl, cyclohexyl, thiazol-2-ylmethyl, cycloheptyl, 2-ethylbutyl, pyrazol-1-yl-methyl, 2,4,6-trifluorobenzyl, indol-3-ylmethyl, N-phenyl-N-methylaminomethyl, methyl, 4-methylindol-3-ylmethyl, or hydrogen;
R^{2a} is hydrogen or R² and R^{2a} together with the carbon atom to which they are attached form cycloheptyl,
R³ is ethyl, n-propyl, n-butyl;
R⁴ is benzoxazol-2-yl, oxazolo[4,5-b]pyridin-2-yl, 2-pyridin-3-yl-[1,3,4]-oxadiazol-5-yl, 2-pyridin-4-yl-[1,3,4]-oxadiazol-5-yl, 2-phenyl-[1,3,4]-oxadiazol-5-yl, 2-ethyl-[1,3,4]-oxadiazol-5-yl, 2-phenyl-[1,3,4]-oxadiazol-5-yl, or 2-tert-butyl-[1,3,4]-oxadiazol-5-yl; and
R¹⁰ and R¹¹ together with the carbon atom to which they are attached form —C═O.

6. Yet another preferred group of compounds of Formula I is that wherein:
R¹ is 2′-chlorobiphen-4-yl, 2′,3-dichlorobiphenyl-4-yl, 2′,6′-dichlorobiphen-4-yl, 2′,6′-dimethylbiphen-4-yl, 2′-methylbiphen-4-yl, 2′-fluorobiphen-4-yl; 4-trifluoromethoxyphenyl, 4-(2-butyl)phenyl, 3,5-diphenylphenyl, 2,3-diphenylthiophen-5-yl, 2-2-methylphenyl)furan-5-yl, 2-(2-methoxyphenyl)furan-5-yl, 3-methoxy-2-(2-methylphenyl)thiophen-4-yl, 3-methoxy-2-(2-methoxyphenyl)thiophen-4-yl, 2,3-di(2-methoxyphenyl)thiophen-5-yl, 3,5-di(2-methoxyphenyl)phenyl, 3,5-di(3-methoxyphenyl)phenyl, 3,5-di(thiophen-3-yl)phenyl, 3,5-(pyridin-4-yl)phenyl, 2,3-di(2-methylphenyl)thiophen-5-yl, 4-tert-butylphenyl, 2,3-di(furan-2-yl)thiophen-5-yl, 3,5-di(furan-2-yl)phenyl, 4-(2-methylphenyl)thiophen-2-yl, 4-(2-methoxyphenyl)thiophen-2-yl, 2′-chlorobiphen-3-yl, 2′-methyl-4-chlorobiphenyl-3-yl, 3,5-di(2-chlorophenyl)phenyl, 2,3-di(2-chlorophenyl)thiophen-5-yl, 1-(4-aminosulfonylphenyl)-5-(4-chlorophenyl)pyrazol-3-yl, 2-(2,6-dichlorophenyl)furan-5-yl, 2,3-diphenylthiophen-5-yl, 4,5-diphenylthiazol-2-yl, 3-trifluoromethyl-1-methylthieno[2,3-c]pyrazol-5-yl, 3-methylbiphen-4-yl, 2′-methoxybiphen-4-yl, 2′-trifluoromethylbiphen-4-yl, 2′-methyl-3-chlorobiphen-4-yl, 2-(2-chlorophenyl)pyridin-5-yl, 2-(2,6-dichlorophenyl)pyridin-5-yl, 2-(2-trifluoromethyl-phenyl)pyridin-5-yl, 4-(3-methylpyridin-2-yl)phenyl, 2-(2-chlorophenyl)-3-chloropyridin-5-yl, 4′-carboxy-2′-chlorobiphen-4-yl, 4′-carboxy-2′-fluorobiphen-4-yl, 4′-carboxy-2′-methylbiphen-4-yl, 5′-carboxy-2′-methylbiphen-4-yl, 4-(3-methylpyridin-2-yl)phenyl, 5′-carboxy-2′-chlorobiphen-4-yl, 2-(4-carboxy-2-chlorophenyl)pyridin-5-yl, 2-(5-carboxy-2-chlorophenyl)-pyridin-5-yl, 4-(5-carboxy-2-methylthiophen-3-yl)phenyl, 3-chloro-2-(2,6-dichlorophenyl)-pyridin-5-yl, 3-(2-chlorophenyl)isoxazol-5-yl or 4-(3-methoxy-phenyl)thiophen-2-yl. More preferably, R¹ is 2′-chlorobiphen-4-yl, 2′,3-dichlorobiphenyl-4-yl, 2′,6′-dichlorobiphen-4-yl, 2′,6′-dimethylbiphen-4-yl, 2′-methylbiphen-4-yl, 2′-fluorobiphen-4-yl; 4-trifluoromethoxy-phenyl, 4-(2-butyl)phenyl, 3,5-diphenylphenyl, 2,3-diphenylthiophen-5-yl, 2-(2-methylphenyl)-furan-5-yl, 2-(2-methoxyphenyl)furan-5-yl, 3-methoxy-2-(2-methylphenyl)thiophen-4-yl, 3-methoxy-2-(2-methoxyphenyl)thiophen-4-yl, 2,3-di(2-methoxyphenyl)thiophen-5-yl, 3,5-di(2-methoxyphenyl)phenyl, 3,5-di(3-methoxyphenyl)phenyl, 3,5-di(thiophen-3-yl)phenyl, 3,5-di(pyridin-4-yl)phenyl, 2,3-di(2-methylphenyl)thiophen-5-yl, 4-tert-butylphenyl, 2,3-di(furan-2-yl)thiophen-5-yl, 3,5-di(furan-2-yl)phenyl, 4-(2-methylphenyl)thiophen-2-yl, 4-(2-methoxyphenyl)-thiophen-2-yl, 2′-chlorobiphen-3-yl, 2′-methyl-4-chlorobiphenyl-3-yl, 3,5-di(2-chlorophenyl)-phenyl, 2,3-di(2-chlorophenyl)thiophen-5-yl, 1-(4-aminosulfonylphenyl)-5-(4-chlorophenyl)-pyrazol-3-yl, 2-(2,6-dichlorophenyl)furan-5-yl, 2,3-diphenylthiophen-5-yl, 4,5-diphenylthiazol-2-yl, 3-trifluoromethyl-1-methylthieno[2,3-c]pyrazol-5-yl, 2-(2-chlorophenyl)pyridin-5-yl, 2-(2′,6′-dichlorophenyl)pyridin-5-yl, 2-(2-trifluoromethylphenyl)-pyridin-5-yl, 4-(3-methylpyridin-2-yl)phenyl, 2-(2-chlorophenyl)-3-chloropyridin-5-yl, 4′-carboxy-2′-chlorobiphen-4-yl, 4′-carboxy-2′-fluorobiphen-4-yl, 4′-carboxy-2′-methylbiphen-4-yl, 5′-carboxy-2′-chlorobiphen-4-yl, 2-(4-carboxy-2-chlorophenyl)pyridin-5-yl, 2-(5-carboxy-2-chlorophenyl)pyridin-5-yl, 4-(5-carboxy-2methylthiophen-3-yl)phenyl, or 4-(3-methoxy-phenyl)thiophen-2-yl. Even more preferably, R¹ is 2′-chlorobiphen-4-yl, 2′,6′-dichlorobiphen-4-yl, 2-(2-chlorophenyl)pyridin-5-yl, or 2′,3-dichlorobiphen-4-yl.

7. Yet another preferred group of compounds of Formula I is that wherein R² is selected from the group consisting of cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl, benzyl (where the phenyl ring in the benzyl group is substituted at the 2 and 6 positions with groups independently selected from methyl, chloro, fluoro, trifluoromethyl, methoxy, trifluoromethoxy, or difluoromethoxy and at the 4 position with hydrogen, methyl, ethyl, propyl, chloro, fluoro, trifluoromethyl, methoxy, 2-methoxyethyloxy, 2-dimethylaminoethyloxy, trifluoromethoxy, difluoromethoxy, methylthio, methylsulfinyl, methylsulfonyl, cyano, amino, methylamino or dimethylamino). R² is preferably selected from the group consisting of cyclohexyl, cycloheptyl, thiazol-2-ylmethyl, 2-ethylbutyl, pyrazol-1-ylmethyl, 2,4,4-trimethylpentyl, 2-mapth-1-ylpropyl, 2-phenylprop-2-enyl, 2-phenyl-2-methylpropyl, 2-phenylpropyl, 2-(2-methoxyphenyl)propyl, 4-methylindol-3-ylmethyl, 2-(2,5-dimethylphenyl)propyl, benzyloxymethyl, 2-(2,4-dimethylphenyl)-propyl, 2-(2,4-dichlorophenyl)-propyl, 2,6-difluorobenzyl, 2,5-difluorobenzyl, and 2,3-difluorobenzyl.

A number of different preferences have been given above, and following any one of these preferences results in a compound of this invention that is more presently preferred than a compound in which that particular preference is not followed. However, these preferences are generally independent and additive; and following more than one of these preferences may result in a more presently preferred compound than one in which fewer of the preferences are followed. Additional aspects of this invention are disclosed in Applicants' U.S. Provisional Application Ser. Nos. 60/380,311, filed on May 14, 2002, and 60/422,337, filed on Oct. 30, 2002, the disclosures of which are incorporated herein by reference in their entirety.

TABLE I

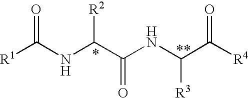

| | Stereochemistry | | | | |
|---|---|---|---|---|---|
| Cpd. # | at (C*, C**) | R¹ | R² | R³ | R⁴ |
| 1 | (S, S) | 2′-Cl-biphenyl-4-yl | 2,6-diF-benzyl | ethyl | benzoxazol-2-yl |
| 2 | (RS, S) | 2′-Cl-biphenyl-4-yl | 2,6-diF-benzyl | ethyl | benzoxazol-2-yl |
| 3 | (RS, S) | 2,3-diphenylthiophen-5-yl | 2,6-diF-benzyl | ethyl | benzoxazol-2-yl |
| 4 | (S, S) | 2′-Cl-biphenyl-4-yl | 2(S)-phenylpropyl | ethyl | benzoxazol-2-yl |
| 5 | (RS, RS) | 2′-Cl-biphenyl-4-yl | 2,6-diF-benzyl | n-propyl | benzoxazol-2-yl |
| 6 | (RS,S) | 2′-Cl-biphenyl-4-yl | 2,6-diF-benzyl | n-butyl | oxazolo[4,5-b]pyridin-2-yl |
| 7 | (AS, RS) | 2′-Cl-biphenyl-4-yl | 2,6-diF-benzyl | n-butyl | benzoxazol-2-yl |
| 8 | (S, S) | 2′-Cl-biphenyl-4-yl | 2,6-diF-benzyl | ethyl | oxazolo[4,5-b]pyridin-2-yl |
| 9 | (RS, S) | 2′-Cl-biphenyl-4-yl | 2,6-diF-benzyl | ethyl | oxazolo[4,5-b]pyridin-2-yl |
| 10 | (RS, S) | 2′-Cl-biphenyl-4-yl | 2,6-diF-benzyl | n-butyl | 2-pyridin-3-yl-[1,3,4]-oxadiazol-5- |
| | | | | | yl |
| 11 | (RS, S) | 2′-Cl-biphenyl-4-yl | 2,6-diF-benzyl | n-butyl | 2-pyridin-4-yl-[1,3,4]-oxadiazol-5- |
| | | | | | yl |
| 12 | (R, S) | 2′-Cl-biphenyl-4-yl | 2(S)-phenylpropyl | ethyl | benzoxazol-2-yl |
| 13 | (RS, S) | 2′-Cl-biphenyl-4-yl | 2,6-diF-benzyl | n-butyl | 2-phenyl-[1,3,4]-oxadiazol-5-yl |
| 14 | (S, S) | 2-(2-Clphenyl)pyridin-5-yl | 2,6-diF-benzyl | ethyl | benzoxazol-2-yl |
| 15 | (S, S) | 2′-Cl-biphen-4-yl | cyclohexyl | ethyl | oxazolo[4,5-b]pyridin-2-yl |
| 16 | (RS, S) | 2′-Cl-biphen-4-yl | 2,6-diF-benzyl | n-propyl | 2-ethyl-[1,3,4]-oxadiazol-5-yl |
| 17 | (RS, S) | 2′-Cl-biphen-4-yl | thiazol-2- | ethyl | benzoxazol-2-yl |
| | | | ylmethyl | | |
| 18 | (S, S) | 2-(2-Cl-phenyl)pyridin-5- | 2(S)-phenylpropyl | ethyl | 2-ethyl-[1,3,4]-oxadiazol-5-yl |
| | | yl | | | |
| 19 | (S, S) | 2,3-diphenylthiophen-5-yl | 2,6-diF-benzyl | ethyl | benzoxazol-2-yl |
| 20 | (S, S) | 2-(2-Clphenyl)pyridin-5-yl | cycloheptyl | ethyl | benzoxazol-2-yl |
| 21 | (S, S) | 2-(2-Clphenyl)pyridin-5-yl | 2(S)-phenylpropyl | ethyl | benzoxazol-2-yl |
| 22 | (RS, RS) | 2′,3-diCl-biphen-4-yl | cyclohexyl | ethyl | oxazolo[4,5-b]pyridin-2-yl |
| 23 | (RS, S) | 4-tert6-butylphenyl | 2-ethylbutyl | ethyl | benzoxazol-2-yl |
| 24 | (S, S) | 2′-Cl-biphen-4-yl | 2-ethylbutyl | ethyl | benzoxazol-2-yl |
| 25 | (S, S) | 2′-Cl-biphen-4-yl | cyclohexyl | ethyl | benzoxazol-2-yl |
| 26 | (S, S) | 2′-Cl-biphen-4-yl | cyclohexyl | n-propyl | 2-ethyl-[1,3,4]-oxadiazol-5-yl |
| 27 | (S, S) | 2′-Cl-biphen-4-yl | pyrazol-1-yl- | ethyl | benzoxazol-2-yl |
| | | | methyl | | |
| 28 | (R, RS) | 2′-Cl-biphen-4-yl | 2(S)-phenylpropyl | ethyl | oxazolo[4,5-b]pyridin-2-yl |
| 29 | (RS, S) | 2′,3-dichlorobiphen-4-yl | cyclohexyl | ethyl | benzoxazol-2-yl |
| 30 | (S, S) | 2′,3-dichlorobiphen-4-yl | cyclohexyl | n-propyl | 2-ethyl-[1,3,4]-oxadiazol-5-yl |
| 31 | (S, S) | 5′-carboxy- | 2,6-diF-benzyl | ethyl | benzoxazol-2-yl |
| | | 2′chlorobiphen-4-yl | | | |
| 32 | (S, S) | 4-morpholin-4-ylphenyl | 2S-phenylpropyl | ethyl | benzoxazol-2-yl |
| 33 | (RS, S) | 2′-chlorobiphen-4-yl | 2,6-diF-benzyl | n-butyl | oxazolo[4,5-b]pyridin-2-yl |
| 34 | (S, S) | 2′-chlorobiphen-4-yl | 2,4,6-triF-benzyl | ethyl | benzoxazol-2-yl |
| 35 | (S, S) | 1-methyl-3- | 2(S)-phenylpropyl | ethyl | benzoxazol-2-yl |
| | | trifluoromethyl-1H-thieno- | | | |
| | | [2,3-c]-pyrazol-5-yl | | | |
| 36 | (S, S) | 2′-chlorobiphen-4-yl | thiazol-2- | ethyl | benzoxazol-2-yl |
| | | | ylmethyl | | |
| 37 | (S, RS) | 2-(2-chlorophenyl)pyridin- | thiazol-2- | ethyl | benzoxazol-2-yl |
| | | 5-yl | ylmethyl | | |
| 38 | (S, S) | 2-(2-chlorophenyl)pyridin- | thiazol-2- | ethyl | benzoxazol-2-yl |
| | | 5-yl | yhnethyl | | |
| 39 | (S, S) | 3-vinylphenyl | 2(S)-phenylpropyl | ethyl | benzoxazol-2-yl |
| 40 | (RS, RS) | 4-phenoxyphenyl | 2,6-diF-benzyl | ethyl | benzoxazol-2-yl |
| 41 | (S, S) | 4-acetylamino-3-methyl- | 2(S)-phenylpropyl | ethyl | benzoxazol-2-yl |
| | | phenyl | | | |
| 42 | (S, S) | 2′-chlorobiphen-4-yl | cyclohexyl | ethyl | 2-phenyl-[1,3,4]-oxadiazol-5-yl |
| 43 | (S, S) | 2′-chlorobiphen-4-yl | indol-3-ylmethyl | ethyl | benzoxazol-2-yl |
| 44 | (S, S) | 2′-chlorobiphen-4-yl | N-phenyl-N- | ethyl | benzoxazol-2-yl |
| | | | methyl- | | |
| | | | aminomethyl | | |
| 45 | (S, S) | 2′,3-dichlorobiphen-4-yl | cyclohexyl | ethyl | 2-phenyl-[1,3,4]-oxadiazol-5-yl |
| 46 | (RS, S) | 2′-chlorobiphen-4-yl | 2,6-diF-benzyl | propyl | 2-tert-butyl-[1,3,4]-oxadiazol-5-yl |
| 47 | (S, S) | 3,5-di(2-methoxyphenyl)- | methyl | ethyl | benzoxazol-2-yl |
| | | phenyl | | | |
| 48 | (RS, S) | 2′-chlorobiphen-4-yl | 4-methylindol-3- | ethyl | benzoxazol-2-yl |
| | | | yl-methyl | | |
| 49 | (S, S) | 5′-carboxy-2′- | methyl | ethyl | benzoxazol-2-yl |
| | | chlorobiphen-4-yl | | | |
| 50 | (R, RS) | 2′-chlorobiphen-4-yl | 2(S)-phenylpropyl | ethyl | 2-phenyl-[1,3,4]-oxadiazol-5-yl |
| 51 | (S, S) | 2′,3-dichlorobiphen-4-yl | methyl | ethyl | benzoxazol-2-yl |
| 52 | (S) | 2′-chlorobiphen-4-yl | H | ethyl | benzoxazol-2-yl |
| 53 | (S) | 5′-carboxy-2′- | H | ethyl | benzoxazol-2-yl |
| | | chlorobiphen-4-yl | | | |

TABLE II

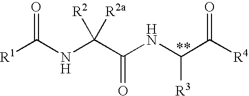

| | Stereochemistry | | | | |
|---|---|---|---|---|---|
| Cpd. # | at (C**) | R¹ | R² + R^{2a} | R³ | R⁴ |
| 54 | (S) | 2′-Cl-biphenyl-4-yl | cycloheptyl | ethyl | oxazolo[4,5-b]pyridin-2-yl |
| 55 | (S) | 2′,3-diCl-biphenyl-4-yl | cycloheptyl | ethyl | oxazolo[4,5-b]pyridin-2-yl |
| 56 | (S) | 2′,3-diCl-biphenyl-4-yl | cycloheptyl | ethyl | benzoxazol-2-yl |

### General Synthetic Scheme

Compounds of this invention can be made by the methods depicted in the reaction schemes shown below.

The starting materials and reagents used in preparing these compounds are either available from commercial suppliers such as Aldrich Chemical Co., (Milwaukee, Wis.) or Bachem (Torrance, Calif.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified, if desired, using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

Compounds of Formula I where R¹, R², R^{2a}, R³, R⁴ are as defined in the Summary of the Invention and R¹⁰ is hydrogen and R¹¹ is hydroxy or R¹⁰ and R¹¹ together with the carbon atom to which they are attached form carbonyl can be prepared as shown in Scheme 1 below.

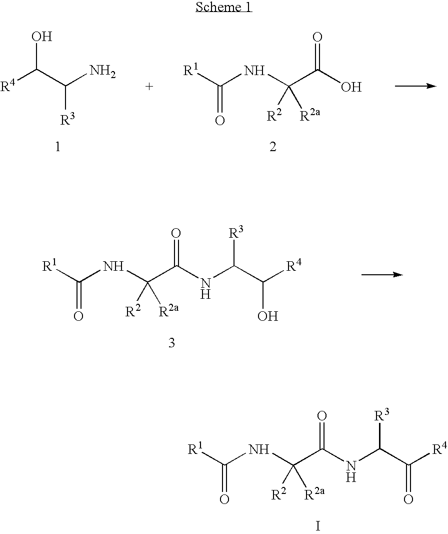

Reaction of an alpha-aminoalcohol compound of formula 1 with an N-acylated amino acid of formula 2 provides a compound of formula 3. The reaction is typically carried out in the presence of a suitable coupling agent e.g., benzotriazole-1-yloxytrispyrrolidinophosphonium hexafluorophosphate (PyBOP®), O-benzotriazol-1-yl-N,N,N′,N′-tetramethyl-uronium hexafluorophosphate (HBTU), O-(7-azabenzotriazol-1-yl)-1,1,3,3-tetramethyl-uronium hexafluorophosphate (HATU), 1-(3-dimethylaminopropyl)-3-ethylcarbodiimide hydrochloride (EDC), or 1,3-dicyclohexylcarbodiimide (DCC), optionally in the presence of 1-hydroxybenzotriazole (HOBT), and a base such as N,N-diisopropylethylamine, triethylamine, N-methylmorpholine, and the like. The reaction is typically carried out at 20 to 30° C., preferably at about 25° C., and requires 2 to 24 h to complete. Suitable reaction solvents are inert organic solvents such as halogenated organic solvents (e.g., methylene chloride, chloroform, and the like), acetonitrile, N,N-dimethylformamide, ethereal solvents such as tetrahydrofuran, dioxane, and the like. Preferably, the reaction is carried out with HOBt, and EDC in dichloromethane.

Alternatively, this reaction can be carried out by first converting 2 into an active acid derivative such as an acid chloride or succinimide ester and then reacting it with an amine of formula 1. The reaction typically requires 2 to 3 h to complete. The conditions utilized in this reaction depend on the nature of the active acid derivative. For example, if it is an acid chloride derivative of 2, the reaction is carried out in the presence of a suitable base (e.g. triethylamine, diisopropylethylamine, pyridine, and the like). Suitable reaction solvents are polar organic solvents such as acetonitrile, N,N-dimethylformamide, dichloromethane, or any suitable mixtures thereof.

Compounds of formula 1 can be prepared under deprotonation reaction conditions by treating benzoxazole, oxazolo[4,5-b]pyridine, 2-pyridin-3-yloxadiazole, 2-pyridin-4-yl-oxadiazole, 2-phenyloxadiazole, and the like, with a Grignard reagent such as isopropylmagnesium chloride and then reacting the resulting organomagnesium reagent with an alpha-(N-protected amino)aldehyde of formula R³CH(NHPG)CHO, where R³ is as defined in the Summary of the Invention and PG is a suitable amino protecting group (such as tert-butyoxycarbonyl, benzyloxycarbonyl, or benzyl) to provide an N-protected compound of formula 1 after treatment with an aqueous acid or buffer. Removal of the amino protecting group then provides a compound of formula 1.

The addition reaction is typically carried out in an ethereal organic solvent such as tetrahydrofuran, diethyl ether, dioxane, and the like, preferably tetrahydrofuran, at a temperature from about −78° C. to about 40° C. Preferably, the reaction is carried out from about −10° C. to about 40° C., more preferably from about −10° C. to about 10° C. The reaction typically requires an hour to complete. The nucleophilic addition reaction is typically carried out from about −10° C. to about room temperature. Compounds of formula R³CH(NHPG)CHO are prepared from commercially available starting materials by methods well known in the art.

The reaction conditions employed for removal of the amino protecting group depends on the nature of the protecting group. For example, if the protecting group is tert-butoxycarbonyl, it is removed under acid reaction conditions. Suitable acids are trifluoroacetic acid (TFA), hydrochloric acid, and the like. If the protecting group is benzyl or benzyloxycarbonyl, it is removed under catalytic hydrogenation reaction conditions. Suitable catalyst are palladium, platinum, rodium based catalysts and others known in the art. Other suitable reaction conditions for their removal can be found in Greene, T. W.; and Wuts, P. G. M.; Protecting Groups in Organic Synthesis; John Wiley & Sons, Inc. 1999. The reaction is carried out in an inert organic solvent methylene chloride, tetrahydrofuran, dioxane, dimethylformamide, and the like.

Compounds of formula 2 can be prepared by methods well known in the art. Some such procedures are described in PCT Application Publication No. WO 00/55144 the disclosure of which is incorporated herein in its entirety. For example, a compound of formula 2 can be prepared by reacting an amino acid of formula R²CH(NH₂)COOH with an acid derivative of the formula R¹COL where L is a suitable leaving group such as chloro and the like. Specifically, a compound of formula 2 where R² is 2,6-difluorobenzyl and R¹ is 2′-chlorobiphenyl-4-yl can be prepared by reacting 2,6-difluorophenylalanine with 2′-chlorobiphenyl-4-ylcarbonyl chloride in the presence of base such as triethylamine and in a suitable organic solvent such as acetonitrile. Amino acids of the formula R²CH(NH₂)COOH are either commercially available or they can be prepared by methods known in the art. For example, 2-amino-3-(4-methylindol-3-yl)propionic acid can be bought from Bachem. Syntheses of some amino acids are described in working examples below.

Acid derivatives of the formula R¹COL where L is a halogen can be prepared by reacting the corresponding acids with a halogenating agent such as oxalyl chloride, thionyl chloride, and the like. Acids of formula R¹COOH are either commercially available or they can be prepared from commercially available starting materials by methods known in the art. For example, 1-(4-aminosulfonylphenyl)-5-(4-chlorophenyl)pyrazol-3-ylcarboxylic acid and 1-methyl-3-trifluoro-1H-thieno[2,3-c]pyrazol-4-ylcarboxylic acid are commercially available from Bionet.

Oxidation of hydroxy group in 3 is carried out with a suitable oxidizing agent such as Dess-Martin Periodinane in a halogenated organic solvent such as methylene chloride, chloroform, carbon tetrachloride, and the like, or a mixture of TEMPO/bleach then provides a compound of Formula I.

A compound of Formula I can be converted to other compounds of Formula I. For example, a compound of Formula I where R¹⁰ is alkoxy can be prepared from a corresponding compound of Formula I where R¹⁰ is hydroxy under alkylating reaction conditions. A compound of Formula I where R¹⁰ and R¹¹ together with the carbon atom to which they are attached form C═O can be reacted with a diol such a ethylene glycol to form a compound where R¹⁰ and R¹¹ together form —O—(C₂)allkylene-O—.

### Pharmacology and Utility

The compounds of the invention are cysteine protease inhibitors. In particular the compounds of the invention inhibit the activity of cathepsins B, L, K, F and/or S and, as such, are useful for treating diseases in which cathepsin B, L, K, F and/or S activity contributes to the pathology and/or symptomatology of the disease. For example, the compounds of the invention are useful in treating tumor invasion and metastasis, in particular as anti-angiogenic agents, rheumatoid arthritis, osteoarthritis, pneumocystis carinii, acute pancreatitis, inflammatory airway disease, atherosclerosis, restenosis, and bone and joint disorders. Furthermore, the compounds of the invention are useful in treating bone resorption disorders, e.g., osteoporosis. The compounds of the invention also are useful in treating autoimmune disorders, including, but not limited to juvenile-onset diabetes, multiple sclerosis, pemphigus vulgaris, Graves disease, myasthenia gravis, systemic lupus erythematosus, rheumatoid arthritis and Hashimoto's thyroiditis. The compounds of the invention also are useful in treating allergic disorders, including, but not limited to asthma; and allogeneic immune reponses, including, but not limited to, organ transplants or tissue grafts.

The cysteine protease inhibitory activities of the compounds of the invention can be determined by methods known to those of ordinary skill in the art. Suitable in vitro assays for measuring protease activity and the inhibition thereof by test compounds are known. Typically, the assay measures protease-induced hydrolysis of a peptide-based substrate. Details of assays for measuring protease inhibitory activity are set forth in Biological Examples 1-5, infra.

### Administration and Pharmaceutical Compositions

In general, compounds of Formula I will be administered in therapeutically effective amounts via any of the usual and acceptable modes known in the art, either singly or in combination with another therapeutic agent. A therapeutically effective amount may vary widely depending on the severity of the disease, the age and relative health of the subject, the potency of the compound used and other factors. For example, therapeutically effective amounts of a compound of Formula I may range from about 10 micrograms per kilogram body weight (μg/kg) per day to about 20 milligram per kilogram body weight (mg/kg) per day, typically from about 100 μg/kg/day to about 10 mg/kg/day. Therefore, a therapeutically effective amount for a 80 kg human patient may range from about 1 mg/day to about 1.6 g/day, typically from about 1 μg/day to about 100 mg/day. In general, one of ordinary skill in the art, acting in reliance upon personal knowledge and the disclosure of this Application, will be able to ascertain a therapeutically effective amount of a compound of Formula I for treating a given disease.

The compounds of Formula I can be administered as pharmaceutical compositions by one of the following routes: oral, systemic (e.g., transdermal, intranasal or by suppository) or parenteral (e.g., intramuscular, intravenous or subcutaneous). Compositions can take the form of tablets, pills, capsules, semisolids, powders, sustained release formulations, solutions, suspensions, elixirs, aerosols, or any other appropriate composition and are comprised of, in general, a compound of Formula I in combination with at least one pharmaceutically acceptable excipient. Acceptable excipients are non-toxic, aid administration, and do not adversely affect the therapeutic benefit of the active ingredient. Such excipient may be any solid, liquid, semisolid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, and the like. Liquid and semisolid excipients may be selected from water, ethanol, glycerol, propylene glycol and various oils, including those of petroleum, animal, vegetable or synthetic origin (e.g., peanut oil, soybean oil, mineral oil, sesame oil, or the like). Preferred liquid carriers, particularly for injectable solutions, include water, saline, aqueous dextrose and glycols.

The amount of a compound of Formula I in the composition may vary widely depending upon the type of formulation, size of a unit dosage, kind of excipients and other factors known to those of skill in the art of pharmaceutical sciences. In general, a composition of a compound of Formula I for treating a given disease will comprise from 0.01 wt % to 10 wt %, preferably 0.3 wt % to 1 wt %, of active ingredient with the remainder being the excipient or excipients. Preferably the pharmaceutical compositions are administered in a single unit dosage form for continuous treatment or in a single unit dosage form ad libitum when relief of symptoms is specifically required. Representative pharmaceutical formulations containing a compound of Formula I are described below.

### EXAMPLES

The following preparations and examples are given to enable those skilled in the art to more clearly understand and to practice the present invention. They should not be considered as limiting the scope of the invention, but merely as being illustrative and representative thereof.

### SYNTHETIC EXAMPLES

### General Procedures

### Example A

### Synthesis of 2(S)-amino-1-(3-phenyl-[1,2,4]oxadiazol-5-yl)-butan-1-ol

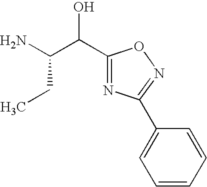

3-tert-Butoxycarbonylamino-2-hydroxy-pentanoic acid (500 mg, 2.14 mmol) was combined with EDC (600 mg, 3.14 mmol), HOBt (600 mg, 3.92 mmol), and N-hydroxy-benzamidine (292 mg, 2.14 mmol). Dichloromethane (10 mL) was added and then 4-methylmorpholine (1 mL). The mixture was stirred at ambient temperature for 16 h. After dilution with ethyl acetate (200 mL), the solution was washed with water (30 mL), saturated aqueous NaHCO₃ solution and brine, dried with MgSO₄, filtered, and evaporated under vacuum. The crude product was dissolved in pyridine (10 mL) and heated at 80° C. for 15 h. The pyridine was evaporated under vacuum and the residue was purified by flash chromatography on silica gel (eluent: ethyl acetate) to yield (290 mg 0.83 mmol). The oxadiazole (145 mg, 0.41 mmol) was dissolved in CH₂Cl₂ (4 mL) and TFA (4 mL) was added. After stirring for 1 h, the mixture was evaporated to dryness to yield 2(S)-amino-1-(3-phenyl-[1,2,4]oxadiazol-5-yl)-butan-1-ol as a TFA salt.

### Example B

### Synthesis of 2(RS)-benzyloxycarbonylamino-4(RS)-(2-methoxyphenyl)pentanoic acid

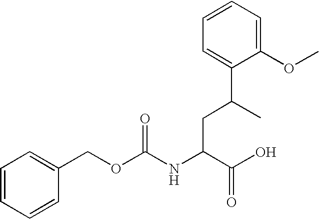

To d,l-2-methoxy-α-methylbenzyl alcohol (0.5 g, 3.29 mmol) was added 48% aq. HBr (2 mL) and the reaction mixture was stirred rapidly for 1.5 h. The reaction mixture was diluted with hexane (30 mL), washed with water, dried with MgSO₄, filtered, and evaporated under vacuum. The crude d,l-2-methoxy-α-methylbenzyl bromide was added to a solution of tributyltin hydride (0.67 mL, 2.49 mmol), Z-dehydroalanine methyl ester (0.25 g, 1.06 mmol), and 2,2′-azobisisobutyronitrile (15 mg, 0.09 mmol) in benzene (5 mL). The reaction mixture was heated at 80° C. under a nitrogen atmosphere for 5 h. Benzene was removed under vacuum and the residue was dissolved in methanol (20 mL). 2N KOH (5 was added and the mixture was rapidly stirred at room temperature over night. Methanol was removed under vacuum and the residue was diluted with water (20 mL). The aqueous solution was washed with ether to remove the tin by products. The aqueous layer was acidified with 6 N HCl (aq.) and the product was extracted with ethyl acetate. The combined organic layers were washed with brine, dried with MgSO₄, filtered, and evaporated under vacuum to give 2-benzyloxy-carbonylamino-4-(2-methoxyphenyl)pentanoic acid (190 mg, 0.53 mmol) as a mixture of diastereomers in sufficiently pure form to be used without further purification. MS: (M⁺+H) 358, (M⁺−H) 356.

Following the procedure described above, and utilizing appropriate starting materials the following amino acids were prepared:
2-benzyloxy-carbonylamino-4-(2-methoxyphenyl)hexanoic acid;
2-benzyloxy-carbonylamino-4-(4-fluorophenyl)pentanoic acid;
2-benzyloxy-carbonylamino-4-(4-chlorophenyl)pentanoic acid;
2-benzyloxy-carbonylamino-4-(4-methoxyphenyl)pentanoic acid;
2-benzyloxy-carbonylamino-4-(2-trifluoromethylphenyl)pentanoic acid;
2-benzyloxy-carbonylamino-4-(3-trifluoromethylphenyl)pentanoic acid;
2-benzyloxy-carbonylamino-4-(napth-1-yl)pentanoic acid;
2-benzyloxy-carbonylamino-4-(2,6-dimethylphenyl)pentanoic acid;
2-benzyloxy-carbonylamino-4-(2,4-difluorophenyl)pentanoic acid;
2-benzyloxy-carbonylamino-4-(2,4-dimethylphenyl)pentanoic acid;
2-benzyloxy-carbonylamino-4-(2,5-dimethylphenyl)pentanoic acid; and
2-benzyloxy-carbonylamino-4-(2,4-dichlorophenyl)pentanoic acid.

The benzyloxycarbonyl group can be removed as described in Example C below to give the corresponding free amino acid.

### Example C

### Synthesis of 2(S)-2,6-difluorophenylalanine

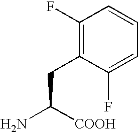 Step 1

N-(Benzyloxycarbonyl)-α-phosphonoglycine trimethyl ester (Aldrich No. 37,635-3; 6.7 g, 20 mmol) and 1,8-diazabicyclo[5,4,0]undec-7-ene (Aldrich No. 13, 900-9; 3.3 mL, 22 mmol) were dissolved in methylene chloride (11 mL) and stirred at room temperature for 15 min., and then cooled to <−30° C. A solution of 2,6-difluorobenzaldehyde (1.9 mL, 20 mmol) in methylene chloride (25 mL) was added to the reaction mixture dropwise over 20 min. The reaction mixture was stirred for another 20 min., and then allowed to warm up to room temperature for 30 min. The reaction mixture was then poured into ethyl ether (300 mL) and washed with 1 N HCl, brine and dried over MgSO₄. Rotary evaporation gave 2-benzyloxycarbonylamino-3-(2,6-difluorophenyl)acrylic acid methyl ester. This crude product was purified by chromatography on a Medium Pressure Liquid Column (MPLC) eluting with 20% ethyl acetate/80% hexane to give pure product (5 g, 72% yield, liquid).

Step 2

A mixture of 2-benzyloxycarbonylamino-3-(2,6-difluorophenyl)acrylic acid methyl ester (14.4 mmol), and catalyst, (+)-1,2-bis-[(2S, 5S)2,5-diethylphopholano]benzene (cyclooctadiene)rhodium (1) trifluoromethanesulfonate (Strem. Chemical No. 45-0151; 104 mg, 0.14 mmol) was dissolved in ethanol (150 mL). Hydrogenation was performed at 50 psi H₂ at room temperature over 2 days. The solvent was then removed by rotary evaporation to give 2(S)-benzyloxycarbonylamino-3-(2,6-difluorophenyl)propionic acid methyl ester.

Step 3

2(S)-Benzyloxycarbonylamino-3-2,6-difluorophenyl)propionic acid methyl ester (5 g, 14.4 mmol) was dissolved in methanol (60 mL) and cooled on ice. 1 N NaOH (22 mL, 22 mmol) was added dropwise over 15 min. The reaction mixture was removed from cooling and continue stirring at room temperature for 4 h. The solvent was then removed by rotary evaporation. The residue was treated with water (100 mL) and then with 1 N HCl to adjust the pH to 4. The product was extracted with ethyl acetate (300 mL, 200 mL). Evaporation of the solvent and crystallization of the residue from methylene chloride/hexane gave 2(S)-benzyloxycarbonylamino-3-(2,6-difluorophenyl)propionic acid (4.6 g, 13.7 mmol, 94% yield).

Step 4

2(S)-Benzyloxyrarbonylamino-3-2,6-difluorophenyl)-propionic acid was hydrogenated at 50 psi in ethanol (25 mL) in the presence of 5% palladium on activated carbon (600 mg) for 24 h. The catalyst was removed by filtration through celite and the solvent evaporated to give a residue which was crystalized from ethyl ether to give 2(S)-2,6-difluorophenylalanine (2.2 g, 11 mmol, 80% yield). ¹H NMR (DMSO-d₆): δ 7.28 (m, 1H), 7.0 (t, J=7.6 Hz, 2H), 2.77 (m, 2H). MS: 202.2 (M+1), 199.7(M−1).

### Example D

### Synthesis of 2(RS)-amino-4(RS)-6,6-trimethylheptanoic acid

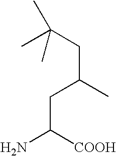 Step 1

To a mixture of the 3,5,5-trimethylhexanal (17.4 mL, 0.10 mol), ammonium chloride (53.5 g, 0.205 mol) and diethyl ether (113 mL) was added sodium cyanide (7.35 g, 0.15 mol) in water (38 mL). The reaction mixture was allowed to stir vigorously for 16 h. The layers were separated. The aqueous layer was extracted with diethyl ether. The combined organic layer was then extracted with 1 N HCl. Saturated sodium bicarbonate was then added until 1-cyano-3,5,5-trimethyl-hexylamine was completely precipitated. Vacuum filtration and washing with 5 mL ice cold water followed by lyophilization gave 1-cyano-3,5,5-trimethylhexylamine (5.805 g, 0.034 mol, 34.5%) as a white solid.

Step 2

1-Cyano-3,5,5-trimethylhexylamine (1.02 g, 5.0 mmol) was treated with 6 N HCl (10 mL) and heated at reflux for 30 h. The reaction mixture was allowed to cool to room temperature. Water (50 mL) was added, and the mixture was washed with diethyl ether.

The aqueous layer was basified to pH 8.5 with 2 M KOH. A white precipitate formed which was collected by vacuum filtration and lyophilized to give 2(RS)-amino-4(RS),6,6-trimethyl-heptanoic acid (364 mg).

### Example E

### Synthesis of 2(RS)-amino-4-methyl-4-phenylpentanoic acid

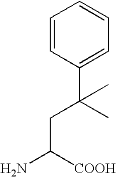 Step 1

4-Methyl-4-phenyl-1-pentene was prepared by reacting 2-phenyl-2-propanol with 3-(trimethylsilyl)propene by the method of Cella, J. Org. Chem., 1982, 47, 2125-2130.

Step 2

4-Methyl-4-phenyl-1-pentene was ozonolyzed at −78° C. in dichloromethane followed by dimethyl sulfide quenching to give crude product which was purified by silica gel chromatography to give 3-methyl-3-phenylbutanal which was then converted to the title compound by proceeding as described in Example D above.

### Example F

### Synthesis of 2(S)-amino-4-phenylpent-4-enoic acid

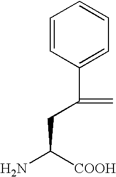 Step 1

Methyl triphenylphosphonium bromide (1.12 g, 3.14 mmol, 2.0 equiv.) was dissolved in THF (15 mL) and cooled to 0° C. Sodium bis(trimethylsilyl)amide (3.14 mL) was added and the reaction mixture was stirred for 30 min. 2(S)-Benzyloxycarbonyl-amino-3-benzoylpropionic acid ethyl ester (0.54 g, 1.57 mmol, 1.0 equiv. prepared by procedures outlined in Lin, W., et. al., Synthesis 2001, No. 7, 1007-1009 was dissolved in TBF (5 mL) and added to the reaction. After warming to room temperature, the reaction mixture was quenched with saturated ammonium chloride and partitioned between water and EtOAc. After concentration of the organic phase, purification was carried out with flash chromatography to provide 2-benzyl-oxycarbonylamino-4-phenyl-pent-4-enoic acid ethyl ester. Removal of the benzyloxycarbonyl group as described above, provided the title compound.

### Example G

### Synthesis of 2(RS)-benzyloxycarbonylamino-4-ethylhexanoic acid

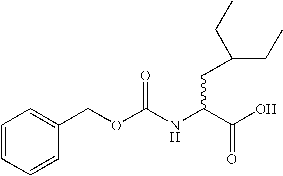 Step 1

A mixture of 2-benzyloxycarbonylaminomalonic acid diethyl ester (Bladon, C. M. J. Chem. Soc. Perkin Trans. 1990, 1, 1151-1158) (1.237 g), iodo-2-ethylbutane (1.272 g) and lithium hydroxide (0.287 g) in N-methylpyrrolidone (8 mL) was stirred for 2 days at room temperature and then diluted with ice water. The aqueous solution was extracted with ether and the product purified by chromatography on silica gel to give 2-benzyloxycarbonylamino-2-(2-ethylbutyl)malonic acid diethyl ester (0.520 g).

Step 2

A solution of 2-benzyloxycarbonylamino-2-(2-ethylbutyl)malonic acid diethyl ester (0.520 g) in ethanol (5 mL) was treated with sodium hydroxide (2.91 mL, 1 N) and then stirred at room temperature for 8 h. The reaction mixture was diluted with water and acidified with HCl and the product was then extracted with ethyl acetate to give 2-benzyloxycarbonylamino-2-(2-ethylbutyl)malonic acid monoethyl ester (0.461 g).

Step 3

2-Benzyloxycarbonylamino-2-(2-ethylbutyl)malonic acid monoethyl ester was heated at 75° C. in ethanol (5 mL) with sodium hydroxide (5 mL, 1 N) for 3 h and 2-benzyloxycarbonyl-amino-2-(2-ethylbutyl)malonic acid was isolated by extraction of the acidified reaction mixture. 2-Benzyloxycarbonylamino-2-(2-ethylbutyl)malonic acid was heated at 103° C. for 1 h and the resulting residue was purified by column chromatography on silica gel to give 2(RS)-benzyloxycarbonylamino-4-ethyl hexanoic acid (0.220 g).

### Example H

### Synthesis of 2′-chlorobiphenyl-4-carboxylic acid

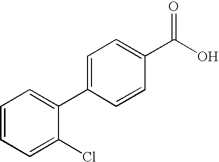 Step 1

4-Bromobenzoic acid ethyl ester (3.91 g, 17.0 mmol, 1.0 equiv.) was combined with Pd tetrakis(triphenylphosphine) (0.98 g, 0.85 mmol, 0.05 equiv.), ethanol (19 mL), and toluene (98 mL). The reaction mixture was stirred for 20 min., at room temperature. To this was added potassium carbonate (11.74 g, 85.0 mmol, 5.0 equiv.) and 2-chlorophenylboronic acid (4.0 g, 25.6 mmol, 1.5 equiv). The suspension was heated to 70° C. for 6 h. The reaction mixture was diluted with ether (400 mL) and extracted with water (400 mL). The organic layer was washed with brine and dried over anhydrous magnesium sulfate. After filtering and concentration the resulting oil was purified by flash chromatography (7% EtOAc/hexanes as eluent to afford 3.16 g of 2′-chlorobiphenyl-4-carboxylic acid ethyl ester.

Step 2

2′-Chlorobiphenyl-4-carboxylic acid ethyl ester was dissolved in MeOH (141 mL). To this was added sodium hydroxide (2.35 g) in water (30 mL). The solution was stirred for 6 h at room temperature, then diluted with 250 mL of water, followed by exatraction with ether (200 mL). The aqueous layer was acidified with conc. hydrochloric acid, extracted with ethyl acetate (300 mL), dried then concentrated to give 2′-chloro-biphenyl-4-carboxylic acid (2.81) as a white solid.

Following the procedure described in Example G above, the following starting materials were prepared:
2′-methylbiphenyl-3-carboxylic acid;
2′-methoxybiphenyl-3-carboxylic acid;
4-chlorobiphenyl-3-carboxylic acid;
4-chloro-2′-methyl-biphenyl-3-carboxylic acid;
4-(2-methylphenyl)thiophen-2-ylcarboxylic acid;
4-(2-methoxyphenyl)thiophen-2-ylcarboxylic acid;
4-(2-chlorophenyl)thiophen-2-ylcarboxylic acid;
2-(2-methylphenyl)-3-methoxythiophen-4-ylcarboxylic acid;
2-(2-methoxyphenyl)-3-methoxythiophen-4-ylcarboxylic acid;
2-(2-methylphenyl)thiophen-5-ylcarboxylic acid;
2-(2-methoxyphenyl)thiophen-5-ylcarboxylic acid;
2-(2-methylphenyl)furan-5-ylcarboxylic acid;
2-(2-methoxyphenyl)furan-5-ylcarboxylic acid;
2-(2,6-dichlorophenyl)thiophen-5-ylcarboxylic acid;
3,5-diphenylbenzoic acid;
3,5-di(2-methoxyphenyl)benzoic acid;
3,5-di(3-methoxyphenyl)benzoic acid;
3,5-dithiophen-3-ylbenzoic acid;
3,5-dipyridin-4-ylbenzoic acid;
3,5-difuran-2-ylbenzoic acid;
3,5-di(2-chlorophenyl)benzoic acid;
2,3-diphenylthiophen-5-carboxylic acid;
2,3-di(2-methoxyphenyl)thiophen-5-carboxylic acid;
2,3-di(2-methylphenyl)thiophen-5-carboxylic acid;
2,3-difuran-2-ylthiophen-5-carboxylic acid;
2,3-di(2-chlorophenyl)thiophen-5-carboxylic acid; and
4,5-diphenylthiazol-2-ylcarboxylic acid;

Starting materials for preparing the above acid via the Suzuki coupling were either commercially available from Aldrich, Frontier, or Lancaster or they were prepared from the synthetic procedure described in Heterocycles, 1995, 41, 1659-1666; Bioorg. Med. Chem. 1999, 7,1559-1566.

### Example I

### Synthesis of 2′,3-dichlorobiphenyl-4-carboxylic acid

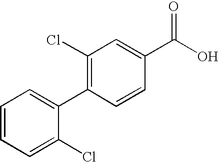 Step 1

3-Chloro-4-hydroxybenzoic acid methyl ester (3.0 g, 16.5 mmol, 1.0 equiv.) was dissolved in dichloromethane (60 mL) and cooled in an ice-water bath. After addition of 2,6-lutidine (9.6 mL), triflic anhydride (4.0 mL) was added dropwise. The reaction mixture was warmed to room temperature and subsequently stirred an additional 16 h. The reaction mixture was diluted with water and ethyl acetate. The organic layer was washed with 1 N HCl, saturated sodium bicarbonate, and dried over anhydrous sodium sulfate and concentrated to give 3-chloro-4-trifluoromethane-sulfonyloxybenzoic acid which was reacted with 2-chlorophenylboronic acid to give 2′,3-dichlorobiphenyl-4-carboxylic acid methyl ester which was converted to the free acid as described above.

Utilizing the procedure described in Example I above, but substituting 3-chloro-4-hydroxybenzoic acid methyl ester with 6-hydroxynicotinic acid provided 2-(2-chlorophenyl)-pyridine-5-carboxylic acid.

### Example J

### Synthesis of 2-(2′-chlorobiphen-4-ylcarbonylamino)-4-phenylpent-4-enoic acid

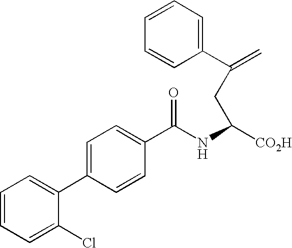 Step 1

α-Methylstyrene was heated with N-bromosuccinamide in carbon tetrachloride to 140° C. until foaming stopped. The reaction mixture was cooled to room temperature and filtered. α-Bromomethylstyrene and β-bromo-α-methylstyrene were obtained by distillation in an 80:20 ratio and used as such in the next step.

Step 2

Sodium ethoxide was generated from sodium metal in ethanol. To this solution was added diethyl malonate. After stirring the reaction mixture for 5 min., a mixture of α-bromomethylstyrene and β-bromo-α-methylstyrene was added and the reaction mixture was heated at 50° C. for 1 h and then allowed to stir at room temperature for 16 h. The reaction mixture was poured into ice water and extracted with ether, dried and concentrated. The crude product was purified from the mixture by silica gel chromatography to give 2-(2-phenylallyl)malonic acid dimethyl ester.

Step 3

2-(2-Phenylallyl)malonic acid dimethyl ester was heated with potassium hydroxide in water and ethanol mixture at 95° C. over 2 h. Ethanol was removed and the basic layer was washed with diethyl ether, acidified and extracted with ethyl acetate, dried and concentrated to give crude 2-(2-phenylallyl)malonic acid which upon heating at 145° C. gave 4-phenylpent-4-enoic acid, which was purified by silica gel chromatography.

Step 4

4-Phenylpent-4-enoic acid was converted to 4-phenylpent-4-enoyl chloride as described in Example 1 below. 4-Phenylpent-4-enoyl chloride was then converted to 2-(2′-chlorobiphen-4-ylcarbonylamino)-4-phenylpent-4-enoic acid by proceeding as described in Example 3, Steps 2-6 described below.

### Example K

### Synthesis of 2(S)-benzyloxycarbonylamino-3-pyrazol-1-ylpropionic acid

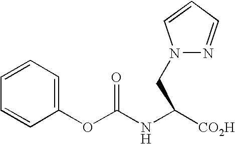

The title compound was prepared by treating S-benzyloxycarbonylserine-β-lactone with pyrazole in acetonitrile at 60° C. for 16 h (see J. Am. Chem. Soc., 1985, 107, 7105-7109).

Following the procedure described above, but substituting pyrazole with 1,2,4-triazole and 1,2,3-triazole provided 2(S)-benzyloxycarbonylamino-3-[1,2,4]-triazol-1-ylpropionic acid and 2(S)-benzyloxycarbonylamino-3-[1,2,3]-triazol-1-ylpropionic acid respectively.

### Example L

### Synthesis of 2(S)-(tert-butoxycarbonyl)amino-1-(oxazolo[4,5-b]pyridin-2-yl)butan-1-ol

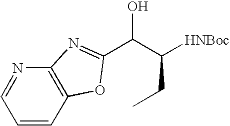 Step 1

A mixture of 2-amino-3-hydroxypyridine (11 g, 100 mmol), triethylorthoformate (80 mL) and p-toluenesulfonic acid (61 mg) was heated at 140° C. for 8 h. Excess triethylorthoformate was removed under vacuum and oxazolo[4,5-b]pyridine was crystalized from ethyl acetate (9 g).

Step 2

In a clean roundbottom flask equipped with stir bar was placed oxazolo[4,5-b]pyridine (600 mg, 5 mmol) in THF (30 mL) and the reaction mixture was cooled to 0° C. under N₂ atomosphere. Isopropylmagnesium chloride (2 M in THF, 2.5 mL, 5 mmol) was added. After stirring for 1 h at 0° C., (S)-2-(tert-butoxycarbonyl)aminobutyraldehyde (573 mg, 3 mmol) in THF (20 mL) was added. The ice bath was removed and the reaction mixture was allowed to warm to room temperature. After 2 h, the reaction mixture was quenched with saturated ammonium chloride solution and concentrated to dryness. The residue was extracted with EtOAc, then washed with brine, dried with anhyd. MgSO₄, 10-filtered and concentrated. The crude product was purified by chromatograph to yield 383 mg of the desired compound.

H¹ NMR (DMSO-d): δ 8.42 (m, 1H), 8.18 (m, 1H), 7.3 (m, 1H), 6.8-6.6 (dd, d, 1H, OH, diastereomer), 6.3-6.02 (d, d, 1H, NH, diastereomer), 4.82-4.5 (m,m, 1H, diastereomer), 1.8-1.3 (m, 2H), 1.2-1.05 (s,s, 9H, diastereomer), 0.89 (m, 3H). MS: 306.2 (M−1), 308.6 (M+1).

### Example M

### Synthesis of 2(S)-(tert-butoxycarbonyl)amino-3-thiazol-2-ylpropionic acid

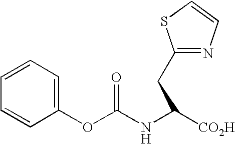

To 2-tert-butoxycarbonylamino-3-thiazol-2-yl-propionic acid methyl ester (500 mg, 1.75 mmol) in a mixture of acetonitrile (6 mL) and 0.2 M aqueous NaHCO₃ (12 mL) was added Alcalase (2.4 L, 0.08 mL), and the solution was stirred vigorously at room temperature for about 2.5 h. The reaction mixture was then evaporated at 30° C. to remove acetonitrile, and the aqueous residue was washed with ether. The aqueous phase was acidified with 6 N HCl to pH 3 and the solution was extracted with ethyl acetate. The combined organic layers were then dried and evaporated to yield 2(S)-tert-butoxycarbonylamino-3-thiazol-2-yl-propionic acid (204 mg).

### Example 1

### Synthesis of N-[1(S)-benzoxazol-2-ylcarbonylpropyl]-2(S)-2′-chlorobiphen-4-ylcarbonylamino)-3-(2,6-difluorophenyl)propionamide (compound 1)

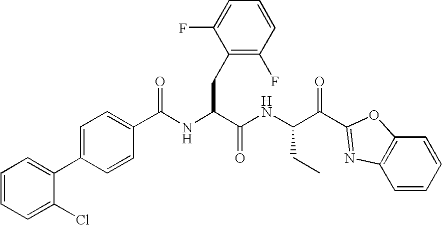 Step 1

A solution of (5S, 6R)-4-tert-butyoxycarbonyl)-5,6-diphenyl-2,3,5,6-tetrahydro-4H-1,4-oxazine-2-one (10.59 g, 0.03 mol) and 2,6-difluorobenzyl bromide (7.038 g, 0.034 mol) in tetrahydrofuran (150 mL) was cooled to −60° C. and then treated with sodium hexamethyldisilazane (32 mL of 1N in tetrahydrofuran) by slow addition over 20 min. The reaction mixture was stirred at −67° C. for 105 min., and then poured into cold water. The product was extracted with ethyl acetate. The extracts were dried and concentrated to 120 mL and cooled to 0° C. Filtration in two crops gave (3S, 5S, 6R)-4-(tert-butyloxycarbonyl)-3-(2,6-difluorophenylmethyl)-5,6-diphenyl-2,3,5,6-tetrahydro-4H-1,4-oxazine-2-one (8.898 g, 62%).

Step 2

A solution of (3S, 5S, 6R)-4-(tert-butyloxycarbonyl)-3-(2,6-difluorophenylmethyl)-5,6-diphenyl-2,3,5,6-tetrahydro-4H-1,4-oxazine-2-one (7.282 g, 0.0152 mol) in methylene chloride (150 mL) was cooled to 0° C. and treated with trifluoroacetic acid (15 mL) and then stirred at room temperature for 4.5 h. The reaction mixture was cooled to 0° C. and treated with triethylamine (27.8 mL). The reaction mixture was then concentrated at reduced pressure, diluted with cold water and the product extracted with ethyl acetate to give (3S, 5S, 6R)-3-(2,6-difluorophenylmethyl)-5,6-diphenyl-2,3,5,6-tetrahydro-4H-1,4-oxazine-2-one (5.86 g) as an oil which was used in the next step without purification.

Step 3

A solution of (3S, 5S, 6R)-3-(2,6-difluorophenylmethyl)-5,6-diphenyl-2,3,5,6-tetrahydro-4H-1,4-oxazine-2-one (5.86 g, 0.152 mol) in tetrahydrofuran (25 mL) and methanol (25 mL) was hydrogenated in the presence of palladium chloride (0.317 g) at 55 psi for 16 h. More (0.100 g) palladium chloride was added to the reaction mixture and the hydrogenation continued for an additional 3 h. The catalyst was removed by filtration, the solvents were removed at reduced pressure and the residue was acidified with 1N aqueous hydrochloric acid. After washing with ethyl acetate the aqueous layer was neutralized to pH 6.9 at 0° C. with 1N sodium hydroxide and then evaporated. The resulting solid was slurried and filtered with methanol (50 mL) twice. Cooling of the methanolic extracts on ice then gave 2(S)-(2,6-difluorophenyl)alanine (1.189 g) as white needles.

Step 4

2′-Chloro-4-biphenylcarboxylic acid (2.77 g, 11.9 mmol) was suspended in ethyl acetate (36 mL). A single drop of N,N-dimethylformamide was added and the suspension cooled in an ice bath. Oxalyl chloride was added dropwise over 5 min., the bath removed and the resulting solution stirred for an additional 20 min. The solvent removed in vacuo and the resulting 2′-chloro-4-biphenylcarbonyl chloride was used immediately without purification.

Step 5

2,6-Difluorophenylalanine (2.4 g, 11.9 mmol) was dissolved in 2 N NaOH (11.9 mL) and dioxane (10 mL) and the solution was cooled in an ice/water bath. A solution of 2′-chloro-4-biphenylcarbonyl chloride in tetrahydrofuran (12 mL) was added concurrently with 2 N NaOH solution (5.9 mL) over 20 min. The ice bath was removed and the reaction mixture was stirred an additional 45 min., after which it was acidified to pH 4 with concentrated HCl. The product was extracted with dichloromethane and the ethereal layer was concentrated to give 2-(2′-chlorobiphen-4-ylcarbonylamino)-3-(2,6-difluorophenyl)propionic acid (3.1 g) which was used in the next step without further purification.

Step 6

To a solution of 2(S)-Boc-aminobutanol in dichloromethane (10 vol) and water (7 vol) were added at 20° C. TEMPO (0.01 equiv.), sodium bromide (1 equiv.) and sodium hydrogen carbonate (3 equiv.). The reaction mixture was stirred at 0° C. and diluted bleach (1.3 equiv., 9 vol) was added over 40 min. The reaction mixture was stirred for 30 min., and then quenched with aq. thiosulfate. After decantation, and extractions (dichloromethane), the organic phase was washed with brine, dried and concentrated in vacuo to dryness, giving 77% of 2(S)-(tert-butoxycarbonyl)amino-butyraldehyde as a low-melting solid.

To a solution of 2(S)-(tert-butoxycarbonyl)aminobutyraldehyde (5.00 g) in toluene (5 vol) was added a solution of Grignard reagent of benzoxazole (prepared at −5° C. from 1.1 equiv. benzoxazole and 1.1 equiv. isopropylmagnesium chloride in THF-toluene 1/1, total 6.5 vol) was added over 30 min. at −5° C. The reaction mixture was stirred for 1 h at 0° C., then 2.5 h at r.t. Quenching with 5% acetic acid(aq.), washings with 5% Na₂CO₃(aq.), then brine and concentration to dryness gave crude 2(S)-(tert-butoxycarbonyl)amino-1-benzoxazol-2-yl-butan-1-ol in a quantitative yield. The residue was diluted with toluene (12 vol), and Florisil® (6 p.) was added. The slurry was filtered on a pad of Florisil® (6 p). Elution by toluene (40 vol) removed the non-polar impurities. Toluene-ethyl acetate (8/2) desorbed the 2(S)-(tert-butoxycarbonyl)-amino-1-benzoxazol-2-ylbutan-1-ol (60-65% yield; red resin).

2(S)-(tert-butoxycarbonyl)amino-1-benzoxazol-2-ylbutan-1-ol (2.5 g) was treated with trimethylchlorosilane (1.4 equiv.) in isopropanol (4 vol) for 5 h at 50° C. Concentration of the reaction mixture to dryness followed by addition of acetone (3 vol) afforded 2(S)-amino-1-benzoxazol-2-yl-butan-1-ol hydrochloride salt as a crystalline product (0.76 g).

Step 7

2-(2′-Chlorobiphen-4-ylcarbonylamino)-3-(2,6-difluorophenyl)propionic acid (5.1 g., 12.26 mmol) was dissolved in acetonitrile (40 mL). HBTU (5.46 g., 14.41 mmol), 2(S)-amino-1-benzoxazol-2-ylbutanol (2.97 g., 14.41 mmol), and N-methylmorpholine (4.34 g, 42.9 mmol) was added. The resulting solution was stirred at ambient temperature for 16 h. The reaction mixture was diluted with ethyl acetate (200 mL) and saturated ammonium chloride (30 mL), then stirred an additional 30 min. The organic layer was removed and the residue was extracted several times with ethyl acetate. The combined organic layers were washed with saturated sodium bicarbonate, saturated sodium chloride, and then dried over anhydrous magnesium sulfate. The solvent was removed and the resulting solid was purified by column chromatography using 50% ethyl acetate/hexanes as eluent to give N-[2(S)-1-benzoxazol-2-yl-1-hydroxybutan-2-yl]-2-(2′-chlorobiphen-4-ylcarbonylamino)-3-(2,6-difluorophenyl)propionamide (2.1 g) as a mixture of diastereomers.

Step 8

N-[2(S)-1-Benzoxazol-2-yl-1-hydroxybutan-2-yl]-2-(2′-chlorobiphen-4-ylcarbonylamino)-3-(2,6-difluorophenyl)propionamide (2.1 g, 3.48 mmol) was dissolved in dichloromethane (30 mL). Dess-Martin periodinane (2.21 g, 5.22 mmol) was added and the reaction mixture was stirred the reaction for 45 min. Na₂S₂O₃ (20 mL, 0.26 M) in saturated sodium bicarbonate and ethyl acetate (200 mL) was added and the reaction mixture was stirred for an additional 30 min. The organic layer was removed and the residue remaining was washed several times with ethyl acetate. The combined organic layers were washed with saturated sodium bicarbonate, saturated sodium chloride, then dried over anhydrous magnesium sulfate. The solvent was removed to give N-[1(S)-benzoxazol-2-ylcarbonylpropyl]-2(S)-(2′-chlorobiphen-4-ylcarbonylamino)-3-(2,6-difluorophenyl)-propionamide (2.1 g) as a white solid (a mixture of diastereomers approx. 8:1).

¹H NMR (400 MHz) (CDCl₃): δ 0.97 (t, J=6.97 Hz, 3H), 1.95 (m, 1H), 2.17 (m, 1H), 3.28 (m, 2H), 4.98 (m, 1H), 5.60 (m, 1H), 6.81 (t, J=8.0 Hz, 2H), 6.90 (d, J=8.8 Hz, 2H), 7.10 (t, J=8.0 Hz, 1H), 7.21 (d, J=7.6 Hz, 1H), 7.32 (m, 31), 7.50 (m, 4H), 7.57 (t, J=6.0 Hz, 1H), 7.68 (d, J=8.0 Hz, 1H), 7.79 (d, J=6.8 Hz, 2H), 7.88 (d, J=8 Hz, 1H).

Proceeding as described in Example 1 above, but substituting appropriate starting materials, the following compounds of the present invention were prepared in Table 1:

Compound 2:

N-[1 (S)-benzoxazol-2-ylcarbonylpropyl]-2(RS)-(2′-chlorobiphen-4-ylcarbonylamino)-3-(2,6-difluorophenyl)propionamide; ¹H NMR (DMSO-d₆): δ 8.69 (t, J=7.6 Hz, 1H), 8.59 (J=8 Hz, 1H), 7.98-7.2 (m, 12H), 6.97 (q, 3H), 5.1-4.9 (m, 1H), 4.9-4.8 (m, 1H), 3.24-2.9 (m, 2H), 1.95 (m, 1H), 1.7 (m, 1H); 0.88-0.83 (t, J=8 Hz, 3H); MS: 600.4 (M−1), 602.4 (M+1).

Compound 3:

N-[1(S)-benzoxazol-2-ylcarbonylpropyl]-2(RS)-(2,3-diphenylthiophen-2-ylcarbonylamino)-3-(2,6-difluorophenyl)propionamide; ¹H NMR (DMSO-d₆): δ 8.8-8.7 (m, 2H), 8.0-6.9 (m, 18H), 5.18 (m, 1H), 4.85 (m, 1H), 3.2-2.9 (m, 2H), 1.95 (m, 1H), 1.70 (m, 1H); 0.93-0.81(t, J=7.6 Hz, 3H); MS: 648.4 (M−1), 650.4 (M+1).

Compound 5:

N-[1(RS)-benzoxazol-2-ylcarbonylbutyl]-2(RS)-(2′-chlorobiphen-4-ylcarbonylamino)-3-(2,6-difluorophenyl)propionamide; ¹H NMR(DMSO-d₆): δ 8.7-8.54 (m, 2H), 7.88-7.76 (m, 13H), 7.04-6.92 (q, 2H), 5.3-5.2 (m, 1H), 4.85 (m, 1H), 3.15-2.95 (m, 2H), 1.9 (m, 1H), 1.65 (m, 1H), 1.4-1.1 (m, 2H), 0.8 (m, 3H); MS: 614.2 (M−1).

Compound 7:

N-[1 (RS)-benzoxazol-2-ylcarbonylpentyl]-2(RS)-(2′-chlorobiphen-4-ylcarbonylamino)-3-(2,6-difluorophenyl)propionamide; ¹H NMR (DMSO-d₆): δ 8.75-8.5 (m, 2H), 8.0-7.15 (m, 2H), 6.95 (q, 2H), 5.3 (m, 1H), 4.9 (m, 1H), 3.23-2.9 (m, 2H), 2.2-1.1 (m, 6H), 0.85 (m, 3H); MS: 628.6 (M−1), 630.4 (M+1).

Compound 19:

N-[1(S)-benzoxazol-2-ylcarbonylpropyl]-2(S)-(2,3-diphenylthiophen-5-ylcarbonylamino)-3-(2,6-difluorophenyl)propionamide; ¹H NMR (DMSO-d₆) δ 8.79-8.71 (m, 2H), 7.95 (d, J=8.4 Hz, 1H), 7.94, (s, 1H), 7.89 (d, J=8.4 Hz, 1H), 7.62 (t, J=8.4 Hz, 1H), 7.50 (t, J=8.4 Hz, 1H), 7.38-7.22 (m, 1H), 7.05-6.95 (m, 2H), 5.23-5.18 (m, 1H), 4.88-4.82 (m, 1H), 3.22-3.15 (m, 1H), 3.04-2.97 (m, 1H), 2.04-1.90 (m, 1H), 1.80-1.68 (m, 1H), 0.95 (t, J=7.2 Hz, 3H). MS: 650 (M+H)⁺.

Compound 40:

N-[1(RS)-benzoxazol-2-ylcarbonylpropyl]-2(RS)-(4-phenoxyphenylcarbonylamino)-3-(2,6-difluorophenyl)propionamide; H¹ NMR (DMSO-d₆): δ 8.7-8.59 (q, 1H), 8.55-8.45 (q, 1H), 8.1-6.95 (m, 16H), 5.15-5.05 (m, 1H), 4.95-4.86 (m, 1H), 3.25-3.1 (m, 1H), 3.05-3 (m, 1H), 2.05-1.9 (m, 1H), 2.9-1.65 (m, 1H), 1.0-0.8 (m, 3H). MS: 582.4 (M−1), 584.4 (M+1).

Compound 52:

N-[1(S)-benzoxazol-2-ylcarbonylpropyl]-2(S)-(2′-chlorobiphen-4-ylcarbonylamino)-acetamide; H¹ NMR (DMSO-d₆): δ 8.85-8.8 (t, J=6.4 Hz, 1H), 8.64-8.62 (d, J=6.4 Hz, 1H), 8.04-7.44 (m, 12H), 5.8-5.4 (m, 1H), 4.1-3.9 (m, 1H), 2.1-2.0 (m, 1H), 1.85-1.7 (m, 1H), 1.02(t, J=7.2 Hz, 3H). MS: 474.2 (M−1), 476.4 (M+1).

### Example 2

### Synthesis of N-[1(S)-benzoxazol-2-ylcarbonylpropyl]-2(S)-(2′-chlorobiphen-4-ylcarbonyl-amino)-4(S)-phenylpentamide and N-[1(S)-benzoxazol-2-ylcarbonylpropyl]-2(R)-2′-chlorobiphen-4-ylcarbonylamino)-4(S)-phenylpentamide (compounds 4 and 12)

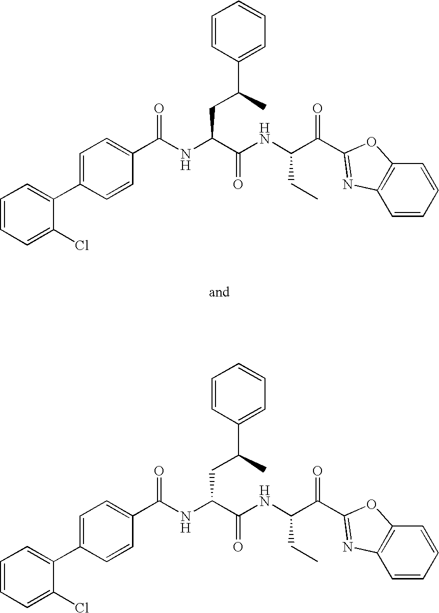 Step 1

2(S)-Phenylpropanol was converted to 1-trifluoromethanesulfonyloxy-2(S)-phenylpropane by the procedure given in Org. Syn. Col. Vol. VIII, p 126.

Step 2

6-Oxo-(2R,3S)-diphenylmorpholine-4-carboxylic acid benzyl ester was converted to 6-oxo-(2R,3S)-diphenyl-5-(2S-phenylpropyl)morpholine-4-carboxylic acid benzyl ester which was then converted to a mixture of 2(R)-amino-4(S)-phenylpentanoic acid and 2(S)-amino-4(S)-phenylpentanoic acid by the methods of Williams, et al., Methods in Molecular Medicine, in Peptidomimetics Protocols; Ed. Kazmierski, W. M. Humana Press Inc.: Totowa, N.J.; Vol. 23, p 339-356 and J. Am. Chem. Soc. 1991, 113, 9276-9286, respectively.

Step 3

A mixture of 2(R)-amino-4(S)-phenylpentanoic acid and 2(S)-amino-4(S)-phenylpentanoic acid was converted to N-[1(S)-benzoxazol-2-ylcarbonylpropyl]-2(S)-(2′-chlorobiphen-4-ylcarbonyl-amino)-4(S)-phenylpentamide and N-[1(S)-benzoxazol-2-ylcarbonylpropyl]-2(R)-(2′-chlorobiphen-4-ylcarbonylamino)-4(S)-phenylpentamide by following the procedure described in Example 1 above. The pure diastereomers were separated by silica gel chromatography using 2-5% diethyl ether in dichloromethane.

2(S)-Amino-4(S)-phenylpentanoic acid can also be prepared as a single (S,S) diastereomer from 6-oxo-(2R,3S)-diphenylmorpholine-4-carboxylic acid benzyl ester as described above by adding all reagents slowly enough to maintain an internal reaction temperature of less than −65° C.

N-[1(S)-Benzoxazol-2-ylcarbonylpropyl]-2(S)-(2′-chlorobiphen-4-ylcarbonyl-amino)-4(S)-phenylpentamide; ¹H NMR (CDCl₃): δ 0.93 (t, J=9 Hz, 3H), 1.29 (d, J=6 Hz, 3H), 1.93 (m, 1H), 2.17 (m, 3H), 2.93 (m, 1H), 4.57 (m, 1H), 5.57 (m, 1H), 6.70 (d, J=9 Hz, 1H), 6.77 (d, J=9 Hz, 1H), 7.17 (m, 1H), 7.30 (m, 7H), 7.47 (m, 4H), 7.53 (m, 1H), 7.67 (m, 3H), and 7.87 (d, J=9 Hz, 1H). MS: 594.5 (M+1); 616.4 (M+23).

N-[1(S)-Benzoxazol-2-ylcarbonylpropyl]-2(R)-(2′-chlorobiphen-4-yl-carbonylamino)-4(S)-phenylpentamide. ¹H NMR (CDCl₃): δ 0.93 (t, J=9 Hz, 3H), 1.33 (d, J=6 Hz, 3H), 1.87 (m, 1H), 2.17 (m, 1H), 2.27 (m, 2H), 3.00 (m, 1H), 4.70 (m, 1H), 5.57 (m, 1H), 6.47 (d, J=9 Hz, 1H), 7.07 (d, J=9 Hz, 1H), 7.20-7.33 (m, 8H), 7.40-7.57 (m, 7H), 7.63 (d, J=9 Hz, 1H), and 7.80 (d, J=9 Hz, 1H). MS: 616.4 (M+23).

### Example 3

### Synthesis of N-[1(S)-benzoxazol-2-ylcarbonylpropyl]-2(S)-(2′-chlorobiphen-4-ylcarbonyl-amino)-4-ethylhexanoamide (compound 24)

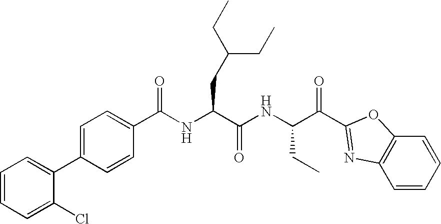 Step 1

A solution of 4-ethylhexanoic acid (prepared by the method described in P. Daud, C. Kaufman, P. Kaufman, Y. Paik, Tet. Lett., 1985, 26, 2279-2282) (11.63 g) in ethyl acetate (150 mL) and dimethyl formamide (2 drops) was treated with oxalyl chloride (10.5 mL) at 0° C. and then stirred at room temperature for 50 min. The solvents were evaporated to give 4-ethylhexanoyl chloride (11.75 g).

Step 2

A solution of 4(S)-phenylmethyl-2-oxazolidone (5.316 g) in THF (60 mL) was cooled to −65° C. and treated with n-butyllithium (20 mL, 1.6 M) over 20 min. A solution of 4-ethylhexanoyl chloride (20.04 g) in THF (5 mL) was added at −65° C. over 20 min. After 30 min., the reaction mixture was quenched in ice water and the product extracted with ethyl acetate to give 3-(4-ethylhexanoyl)-4(S)-phenylmethyl-2-oxazolidone (8.78 g).

Step 3

3-(4-Ethylhexanoyl)-4(S)-phenylmethyl-2-oxazolidone was converted to 3-(2S-azido-4-ethylhexanoyl)-4(S)-phenylmethyl-2-oxazolidone using potassium hexamethyldisilazide and trisyl azide as described by D. A. Evans, T. C. Britton, J. A. Ellman, R. L. Dorow, J. Am. Chem. Soc., 1990, 112, 4011-4030.

Step 4

3-(2S-Azido-4-ethylhexanoyl)-4(S)-phenylmethyl-2-oxazolidone (0.20 g) in methanol (6 mL) was treated with 5% Pd/C (70 mg) and the hydrogenated at 1 atm. When the reaction was complete the reaction mixture was filtered and the methanol evaporated to give 3-(2S-amino-4-ethylhexanoyl)-4(S)-phenylmethyl-2-oxazolidone.

Step 5

3-(2(S)-Amino-4-ethylhexanoyl))-4(S)-phenylmethyl-2-oxazolidone was dissolved in acetonitile and treated with HBTU (285 mg), 2′-chloro-4-biphenyl carboxylic acid (175 mg) and N-methylmorpholine (0.22 mL). After stirring at room temperature for 24 h the reaction mixture was diluted with water and the product extracted with ethyl acetate and purified by chromatography on silica gel to give 3-[2(S)-(2′-chlorobiphen-4-ylcarbonylamino)-4-ethylhexanoyl)]4(S)-phenylmethyl-2-oxazolidone (0.128 g).

Step 6

3-[2(S)-(2′-Chlorobiphen-4-ylcarbonylamino)-4-ethylhexanoyl)]-4(S)-phenylmethyl-2-oxazolidone (0.100 g) in THF (5 mL) was cooled on ice and treated with water (1.25 mL), hydrogen peroxide (30% 1.95 mL) and lithium hydroxide (0.0010 g). The reaction mixture was stirred at room temperature for 90 min. The reaction mixture was quenched with aqueous sodium sulfite and the product isolated from the acidified aqueous layer to give 2(S)-(2′-chlorobiphen-4-ylcarbonylamino)₄-ethylhexanoic acid (0.032 g).

Step 7

2(S)-(2′-Chlorobiphen-4-ylcarbonylamino)-4-ethylhexanoic acid in methylene chloride (2 mL) was treated with 2(S)-amino-1-benzoxazol-2-ylbutanol (25 mg), EDC (26 mg), HOBt (17 mg) and N-methylmorpholine (0.2 mL). The reaction mixture was stirred at room temperature for 35 minutes and then worked up with ethyl acetate and water to give N-[2(S)-1-benzoxazol-2-yl-1-hydroxybutan-2-yl]-2-(2′-chlorobiphen-4-ylcarbonylamino)-4-ethylhexanoamide (21 mg).

Step 8

N-[2(S)-1-benzoxazol-2-yl-1-hydroxybutan-2-yl]-2-(2′-chlorobiphen-4-ylcarbonylamino)-4-ethylhexanoamide (16 mg) was dissolved in methylene chloride (1.5 mL) and treated with Dess-Martin periodinane (16 mg). After stirring 20 min., at room temperature the reaction mixture was quenched with aqueous sodium thiosulfate and the product isolated by extraction with ethyl acetate to give the title compound (8 mg).

### Example 4

### Synthesis of N-[1(S)-benzoxazol-2-ylcarbonylpropyl]-2(S)-(2′-chlorobiphen-4-ylcarbonylamino)-cyclohexylacetamide (compound 25)

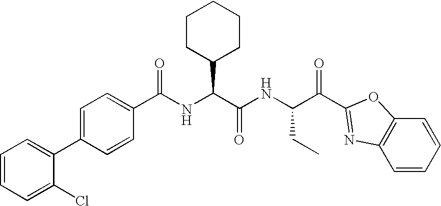 Step 1

To a stirred mixture of tert-butoxycarbonylaminocyclohexylacetic acid (250 mg, 0.97 mmol), 2-amino-1-benzooxazol-2-yl-butan-1-ol TFA salt (0.97 mmol), and HOBt (183 mg, 1.2 mmol) in acetonitrile (5 mL) was added EDC (290 mg, 1.5 mmol) and N-methylmorpholine (0.45 mL) at room temperature. After stirring for 14 h, the reaction mixture was extracted with ethyl acetate. The organic layer was washed with saturated NaHCO₃, brine, dried with MgSO₄ and concentrated to yield {[1-(benzoxazol-2-ylhydroxymethyl)propylcarbamoyl]-cyclohexylmethyl}-carbamic acid tert-butyl ester (400 mg) which was used in the next step without further purification.

Step 2

{[1 (Benzoxazol-2-ylhydroxymethyl)propylcarbamoyl]cyclohexylmethyl}carbamic acid tert-butyl ester (400 mg, 0.9 mmol) and methylene (5 mL) were mixed and TFA (1 mL) was added at room temperature. After stirring for 1 h, the solvent and excess TFA were removed under vacuum to 2-amino-N-[1-(benzoxazol-2-ylhydroxymethyl)propyl]-2-cyclohexylacetamide TFA salt.

Step 3

To a stirred mixture of 2-amino-N-[1-(benzoxazol-2-ylhydroxymethyl)propyl]-2-cyclohexylacetamide TFA salt (0.48 mmol) made as above, 2′-chloro-biphenyl-4-carboxylic acid (111 mg, 0.48 mmol) and HOBt (88 mg, 0.58 mmol) in MeCl₂ (5 mL) was added EDC (139 mg, 0.72 mmol) and N-methylmorpholine (0.32 mL) at room temperature. After stirring for 14 h, the reaction mixture was extracted with ethyl acetate. The organic layer was washed with saturated NaHCO₃, brine, dried with MgSO₄ and concentrated to yield 2′-chlorobiphenyl-4-carboxylic acid {[1-(benzoxazol-2-yl-hydroxy-methyl)propylcarbamoyl]cyclohexylmethyl}-amide (187 mg).

Step 4

2′-Chlorobiphenyl-4-carboxylic acid {[1-(benzoxazol-2-yl-hydroxy-methyl)propyl-carbamoyl]cyclohexylmethyl}amide was dissolved in MeCl₂ (5 mL) and the solution was treated with Dess-Martin periodinane (288 mg, 0.68 mmol) at room temperature. After stirring for 1 h, 5 mL of saturated Na₂S₂O₃—NaHCO₃ were added. After a further 0.5 h, the reaction mixture was extracted with ethyl acetate, washed with brine, dried with MgSO₄ and concentrated. The residue was purified with silica gel column chromatography to yield 100 mg of the title compound.

¹H NMR (DMSO-d₆): δ 8.7 (d, J=12.8 Hz, 1H), 8.28 (d, J=8.4 Hz, 1H), 8.0-7.8 (m, 4H), 7.65-7.38 (m, 8H), 5.17 (m, H), 4.4 (t, 1H), 2.0 (m, 1H), 1.8-1.5 (m, 7H), 1.2-1.0 (m, 5H), 0.98 (t, J=7.2 Hz, 3H). MS: 558.2 (M+1), 580.4 (M+Na).

Compound 15:

Following the procedure described in Example 4 above, but substituting 2-amino-1-benzoxazol-2-ylbutan-1-ol with 2-amino-1-(oxazolo[4,5-b]pyridin-2-yl)butan-1-ol provided N-[1-(S)-oxazolo[4,5-b]pyridin-2-ylcarbonylpropyl]-2(S)-(2′-chlorobiphen-4-ylcarbonylamino)-cyclohexylacetamide. ¹H NMR (DMSO-d₆): δ 8.79 (d, J=5.2 Hz, 1H), 8.7 (m, 1H), 8.86 (m, 2H), 8.28 (d, J=8.4 Hz, 1H), 7.86 (m, 1H), 7.65-7.6 (m, 1H), 7.6-7.55 (m, 1H), 7.5-7.38 (m, 5H), 5.12 (m, 1H), 4.4 (t, J=8.4 Hz, 1H), 2.1-1.9 (m, 1H), 1.85-1.5 (m, 7H), 1.2-1.1 (m, 5H), 0.99 (t, J=6.4 Hz, 3H). MS: 559.3 (M−1), 581.5 (M+Na).

Compound 22:

Following the procedure described in Example 4 above, but substituting 2-amino-1-benzoxazol-2-yl-butan-1-ol with 2-amino-1-(oxazolo[4,5-b]pyridin-2-yl)butan-1-ol and 2′-chlorobiphenyl-4-carboxylic acid with 2′,3-dichlorobiphenyl-4-carboxylic acid provided N-[1(RS)-oxazolo[4,5-b]pyridin-2-ylcarbonylpropyl]-2(RS)-(2′,3-dichlorobiphen-4-ylcarbonylamino)-cyclohexylacetamide. ¹H NMR (DMSO-d₆): δ 8.87 (d, J=4.8 Hz, 1H), 8.29 (d, J=8.8 Hz, 1H), 7.88-7.84 (m, 2H), 7.7-7.3 (m, 6H), 5.03 (m, 1H), 4.4 (t, J=8.4 Hz, 1H), 2.95 (q, J=6 Hz, 2H), 2-1 (m, 15H), 1.28 (t, J=7.6 Hz, 3H), 0.898 (t, J=5.6 Hz, 3H). MS: 551.4 (M−1), 573.4 (M+1).

Compound 26:

Following the procedure described in Example 4 above, but substituting 2-amino-1-benzoxazol-2-ylbutan-1-ol with 2-amino-(2-ethyl-[1.3.4]-oxadiazol-5-yl)pentan-1-ol provided N-[1-(S)-(2-thyl-[1.3.4]-oxadiazol-5-ylcarbonyl)butyl]-2(8)-(2′-chlorobiphen-4ylcarbonylamino)-cyclohexylacetamide. ¹H NMR (DMSO-d₆): δ 9-7.2 (m, 12H), 5.1-5 (m, 1H), 4.64.2 (m, 1H), 2.1-2 (m, 1H), 1.9-0.9 (m, 15H). MS: 593.3 (M+1), 615.3 (M+Na).

### Example 5

### Synthesis of N-[1-(S)-benzoxazol-2-ylcarbonylpropyl]-2(RS)-(2′-chlorobiphen-4-ylcarbonylamino)thiazol-2-ylpropionamide (compound 17)

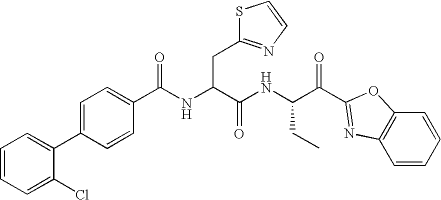 Step 1

To a solution of 2-amino-3-thiazol-2-ylpropionic acid (100 mg, 0.58 mmol) in the mixture of methanol (1 mL) and benzene (5 mL), was added (trimethyl)diazomethane (2 M solution in hexane, 0.76 mL) at room temperature. After 2 h, the solvent was removed under vacuum. The residue was used in the next reaction without further purification.

Step 2

To a stirred solution of 2-amino-3-thiazol-2-ylpropionic acid methyl ester in methylene chloride (5 mL) was added 2′-chlorobiphenyl-4-carboxylic acid (132 mg, 0.57 mmol), HOBt (105 mg, 0.68 mmol), and then EDC (165 mg, 0.86 mmol) and N-methylmorpholine (0.3 mL) at room temperature. After stirring for 14 h, the reaction mixture was extracted with ethyl acetate. The organic layer was washed with saturated NaHCO₃, brine, dried with MgSO₄ and concentrated to yield 2-(2′-chlorobiphenyl-4-carbonylamino)-3-thiazol-2-ylpropionic acid methyl ester.

Step 3

2-(2′-Chlorobiphenyl-4-carbonylamino)-3-thiazol-2-ylpropionic acid methyl ester was dissolved in MeOH (3 mL) and then aq. NaOH was added (1 N, 0.68 mL) at room temperature. After stirring for 1 h, the reaction mixture was acidified with 1 N HCl to pH 2. The product was extracted with ethyl acetate and washed with brine. After drying with MgSO4, the solvent was removed under vacuum and the product was crystallized from ethyl acetate and hexane to yield 176 mg of 2-(2′-chlorobiphenyl-4-carbonylamino)-3-thiazol-2-yl-propionic acid which was then converted to the title compound by following the procedure described in Example 1 above.

¹H NMR (DMSO-d₆): δ 8.86 (m, 2H), 7.96 (m, 1H), 7.88 (m, 2H), 7.69 (t, J=2.4 Hz, 1H), 7.65-7.35 (m, 10H), 5.22 (m, 1H), 5.0 (m, 1H), 3.6-3.4 (m, 21H), 2.0 (m, 1H), 1.77 (m, 1H), 0.98 (m, 3H). MS: 571.2(M−1), 573 (M−1), 595.2 (M+Na).

### Example 6

### Synthesis of N-[1(S)-benzoxazol-2-ylcarbonylpropyl]-2(S)-[2-(2-chlorophenyl)pyridin-5-ylcarbonylamino)-cycloheptylacetamide (compound 20)

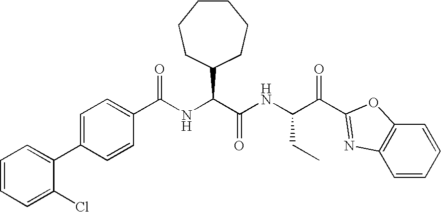

The title compound was prepared following the procedure described in Example 3 above, but substituting 2′chloro-4-biphenylcarboxylic acid with 2-(2-chlorophenyl)pyridinecarboxylic acid and 4-ethylhexanoic acid with 2-cycloheptylacetic acid.

¹H NMR (DMSO-d₆): δ 9.05 (d, J=1.2 Hz, 1H), 8.09 (dd, J=2.4 Hz, J=8 Hz, 1H), 7.816 (d, J=8 Hz, IIH), 7.68 (d, J=7.6 Hz, 1H), 7.6-7.2 (m, 7H), 8.864 (d, J=8 Hz, 1H), 6.592 (d, J=8 Hz, 1H), 5.584 (m, 1H), 4.6 (t, J=7.4 Hz, 1H), 2.25-1.2 (m, 15H), 0.956 (t, J=7.2 Hz, 3H). MS: 572.2(M−1), 573.4 (M−1), 595.5 (M+Na).

### Example 7

### Synthesis of N-[1(S)-benzoxazol-2-ylcarbonylpropyl]-2(S)-[2-(2-chlorophenyl)pyridin-1-yl-carbonylamino]-4(S)-phenylpentamide (compound 21)

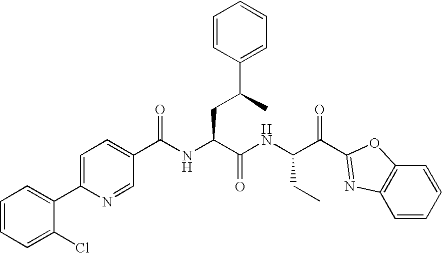

Proceeding as described in Example 1 above, but substituting 2,6-difluorophenylalanine with 2(S)-amino-3(S)-phenylpentanoic acid and 2′-chloro-4-biphenylcarbonyl chloride with 2-(2-chlorophenyl)pyridin-5-ylcarbonyl chloride provided 2-[2-(2-chlorophenyl)pyridin-5-ylcarbonyl-amino]-4(S)-phenylpentanoic acid which was then converted to N-[1(S)-benzoxazol-2-ylcarbonylpropyl]-2(S)-[2-(2-chlorophenyl)pyridin-1-ylcarbonylamino]-4(S)-phenyl-pentamide. MS 596.1 (M+1).

Analytical Data for Other Compound of the Invention is as Follows:

Compound 4:

N-[1(S)-benzoxazol-2-ylcarbonylpropyl]-2(S)-(2′-chlorobiphen-4-ylcarbonylamino)-4(S)-phenylpentamide. ¹H NMR (CDCl₃): δ 7.90 (m, 1H), 7.71 (m, 3H), 7.51 (m, 5H), 7.14-7.40 (m, 8H), 6.73 (m, 1H), 6.64 (m, 1H), 5.62 (m, 1H), 4.58 (m, 1H), 2.98 (m, 1H), 2.21 (m, 3H), 1.92 (m, 1H), 1.33 (m, 3H), 0.99 (m, 3H). MS: 592.2 (M−1), 594.3 (M+1).

Compound 8:

N-[1(S)-oxazolo-[4,5-b]pyridin-2-ylcarbonylpropyl]-2(S)-(2′-chlorobiphen-4-ylcarbonylamino)-3-(2,6-difluorophenyl)propionamide. MS: 603.4 (M+1), 601.5 (M−1).

Compound 9:

N-[1(S)-oxazolo-[4,5-b]-pyridin-2-ylcarbonylpropyl]-2(RS)-(2′-chlorobiphen-4-yl-carbonylamino)-3-(2,6-difluorophenyl)propionamide. MS: 603.4 (M−1), 605.2 (M+1).

Compound 10:

N-[1 (S)-(2-pyridin-3-yl-[1,3,4]-oxadiazol-5-ylcarbonyl)pentyl]-2(RS)-(2′-chlorobiphen-4-ylcarbonylamino)-3-(2,6-difluorophenyl)propionamide. MS: 656.4 (M−1), 658.6 (4+1).

Compound 11:

N-[1(S)-2-pyridin-4-yl-[1,3,4]-oxadiazol-5-ylcarbonyl)pentyl]-2(RS)-(2′-chlorobiphen-4-ylcarbonylamino)-3-(2,6-difluorophenyl)propionamide. MS: 656.4 (M−1), 658.6 (M+1).

Compound 13:

N-[1(S)-(2-phenyl-[1,3,4]xadiazol-5-ylcarbonyl)pentyl]-2(RS)-(2′-chlorobiphen-4-yl-carbonylamino)-3-2,6-difluorophenyl)propionamide. MS: 655.4 (4-1), 679.4 (M+Na).

Compound 14:

N-[1(S)-benzoxazol-2-ylcarbonylpropyl]-2(S)-[2-(2-chlorophenyl)pyridin-5-ylcarbonylamino]-3-(2,6-difluorophenyl)propionamide. MS: 604.3 (M−H).

Compound 16:

N-[1 (s)-(2-ethyl-[1,3,4]-oxadiazol-5-ylcarbonyl)butyl]-2(RS)-(2′-chlorobiphen-4-yl-carbonylamino)-3-(2,6-difluorophenyl)propionamide. ¹H-NMR (DMSO-d₆): 8.76˜8.54 (m, 2H), 7.90˜6.94 (m, 11H), 5.15˜4.95 (m, 1H), 4.83 (m, 1H), 3.26˜2.92 (m, 2H), 1.90˜1.54 (m, 2H), 1.48˜1.14 (m, 5H), 0.94˜0.82 (m, 3H). MS: 581 (M+1).

Compound 18:

N-[1(S)-(2-ethyl-[1,3,4]oxadiazol-5-ylcarbonyl)propyl]-2(S)-[2-(2-chlorophenyl)pyridin-5-ylcarbonylamino]-4(S)phenylpentamide. MS: 575.2 (M+1).

Compound 23:

N-[1(S)-benzoxazol-2-ylcarbonylpropyl]-2(RS)-(4-tert-butylphenylcarbonylamino)-4-ethylhexanoamide. MS: 506.6 (M+1), 504.4 (M−1).

Compound 27:

N-[1(S)-benzoxazol-2-ylcarbonylpropyl]-2(S-(2′-chlorobiphen-4-ylcarbonylamino)-pyrazol-1-ylpropionamide. MS: 5544.5 (M−1), 556.3 (M+1).

Compound 28:

N-[1(RS)-oxazolo-[4,5-b]-pyridin-2-ylcarbonylpropyl]-2(R)-(2′-chlorobiphen-4-yl-carbonylamino)-4(S)-phenylpentamide. MS: 593.6 (M−1), 595.2 (M+1).

Compound 29:

N-[1(S)-benzoxazol-2-ylcarbonylpropyl]-2(RS)2′,3-dichlorobiphen-4-ylcarbonyl-amino)-cyclohexylacetamide. MS: 614.3 (M+Na).

Compound 30:

N-[1(S)-(2-ethyl-[1,3,4]-oxadiazol-5-ylcarbonylbutyl]-2(S)-(2′,3-dichlorobiphen-4-yl-carbonylamino)-cyclohexylacetamide. MS: 607.4 (M+Na).

Compound 31:

N-[1(S)-benzoxazol-2-ylcarbonylpropyl]-2(S)-(5′-carboxy-2′-chlorobiphen-4-ylcarbonylamino)-3-(2,6-difluorophenyl)propionamide. ¹H-NMR (DMSO-d₆): δ 8.81 (m, 1H), 8.62 (m, 1H), 8.04˜9.98 (m, 14H), 5.27 (m, 1H), 4.87 (m, 1H), 3.40˜3.10 (m, 2H), 2.20˜1.65 (m, 2H), 1.03 (t, 3H), 0.94 (m, 3H). MS: 646 (M+1).

Compound 32:

N-[1(S)-benzoxazol-2-ylcarbonylpropyl]-2(S)-(4-morpholin-4-ylphenylcarbonylamino)-4(S)-phenylpentamide. ¹H NMR (CDCl₃): δ 8.660 (d, J=8.0 Hz, 1H), 8.526 (s, 1H), 7.960 (d, J=8.0 Hz, 2H), 7.56-7.59 (m, 1H), 7.521 (d, J=8.2 Hz, 2H), 7.434 (m, 2H), 7.24-7.30 (m, 3H), 7.14-7.18 (m, 1H), 4.624 (m, 1H), 2.83-2.92 (m, 1H), 2.660 (m, 2H), 2.316 (q, J=5.8 Hz, 2H), 2.217 (m, 4H), 2.00-2.19 (m, 1H), 1.80-1.95 (m, 3H), 1.231 (d, J=6.4 Hz, 3H), 0.971 (t, J=5.8 Hz, 3H). MS: 567.4 (M−1), 569.3 (M+1), 591.3 (M+Na).

Compound 33:

N-[1(S)-oxazolo[4,5-b]pyridin-2-ylcarbonylpentyl]-2(RS)-(2′-chlorobiphen-4-ylcarbonylamino)-3-(2,6-difluorophenyl)propionamide. ¹H NMR (DMSO-d₆): δ 8.8-8.55 (m, 3H), 8.45-8.34 (m, 1H), 8.0-7.4 (m, 9), 7.35-7.2 (m, 1H), 7.1-6.9 (q, 2H), 5.3-5.12 (m, 1H), 4.95-4.78 (m, 1H), 3.3-3.1 (m, 1H), 3.1-2.9 (m, 1H), 2.2-1.8 (m, 1H), 1.8-1.55 (m, 1H), 1.5-1.1 (m, 4H), 0.9-0.8 (m, 3H). MS: 631.2 (M−1), 633.4 (M+1).

Compound 34:

N-[1(S)-benzoxazol-2-ylcarbonylpropyl]-2(8-(2′-chlorobiphen-4-ylcarbonylamino)-3-(2,4,6-trifluorophenyl)propionamide. ¹H NMR (400 MHz, DMSO-d₆): δ 7.88-7.09 (m, 12H), 6.55 (m, 2H), 5.62 (m, 1H), 5.10 (m, 1H), 3.28 (dd, J=14.0, J=5.2 Hz, 1H), 3.47 (dd, J=14.0, J=9.2 Hz, 1H), 2.16 (m, 1H), 1.93 (m, 1H), 1.00 (t, J=7.2 Hz, 3H). MS: 620.3 (MH⁺).

Compound 35:

N-[1-(S)-benzoxazol-2-ylcarbonylpropyl]-2(S)-1-methyl-3-trifluoro-1H-thieno-[2,3-c]-pyrazol-5-ylcarbonylamino)-4(S)-phenylpentamide. ¹H NMR (CDCl₃): δ 7.79 (d, J=8.4 Hz, 1H), 7.60 (d, J=8.4 Hz, 1H), 7.35-7.50 (m, 2H), 7.09-7.28 (m, 5H), 6.96 (s, 1H), 6.50 (d, J=7.6 Hz, 1H), 6.38 (d, J=8.0 Hz, 1H), 5.49 (m, 1H), 4.38 (m, 1H), 3.95 (s, 3H), 2.86 (m, 1H, 2.10 (m, 3H), 1.82 (m, 1H), 1.21 (d, J=7.2 Hz, 3H)), 0.89 (t, J=8.0 Hz, 3H). MS: 610.2 (M−1), 612.3 (M+1), 634.6 (M+Na).

Compound 36:

N-[1(S)-benzoxazol-2-ylcarbonylpropyl]-2(S)-(2′-chlorobiphen-4-ylcarbonylamino)-3-thiazol-2-ylpropionamide. ¹H NMR (400 MHz, DMSO-d₆): δ 8.42 (d, J=6.8 Hz, 1H), 8.30 (d, J=6.8 Hz, 1H), 7.96 (d, J=8.4 Hz, 2H), 7.76-7.26 (m, 10H) 5.53 (m, 1H), 5.21 (m, 1H), 3.83 (dd, J=15.6, J=4.0 Hz, 1H), 3.47 (dd, J=15.6, 6.8 Hz, 1H), 2.10 (m, 1H), 1.91 (m, 1H), 0.92 (t, J=7.2 Hz, 3H). MS: 573.2(MH⁺).

Compound 37:

N-[1(RS)-benzoxazol-2-ylcarbonylpropyl]-2(S)-[2-(2-chlorophenyl)pyridin-5-ylcarbonylamino)-3-thiazol-2-ylpropionamide. ¹H NMR (DMSO-d₆): δ 9.08 (d, 1H), 9.02 (m, 1H), 8.88 (d,d, 1H), 8.21 (m, 1H), 7.98-7.93 (m, 1H), 7.89-7.83 (m, 1H), 7.76-7.72 (m, 1H), 7.68 (d, 1H), 7.64-7.43 (m, 7H), 5.25-5.17 (m, 1H), 5.08-4.96 (m, 1H), 3.7-3.5 (m, 1H), 3.5-3.3 (m, 1H), 2.1-1.95 (m, 1H), 1.85-1.65 (m, 1H), 0.99 (t, 3H). MS: 572.1 (M−1), 574.2 (M+1).

Compound 38:

N-[1-(S)-benzoxazol-2-ylcarbonylpropyl]-(2S)-[2-(2-chlorophenyl)pyridin-5-yl-carbonylamino)-3-thiazol-2-ylpropionamide. ¹H NMR (400 MHz, DMSO-d₆): δ 9.24 (dd, J=2.8, 0.8 Hz, 1H), 8.65 (d, J=6.8 Hz, 1H), 8.28-7.25 (m, 11H), 5.54 (m, 1H), 5.21 (m, 1H), 3.84 (dd, J=15.6, 4.0 Hz, 1H), 3.46 (dd, J=15.6, 6.8 Hz, 1H), 2.14 (m, 1H), 1.91 (m, 1H), 0.92 (t, J=8.0 Hz, 3H). MS: 574.1 (MH⁺).

Compound 39:

N-[1(S)-benzoxazol-2-ylcarbonylpropyl]-2(S)-(3-vinylphenylcarbonylamino)-4S-phenylpentamide. ¹H NMR (CDCl₃): δ 7.40-7.53 (m, 3H), 6.90-7.40 (m, 9H), 6.55-6.80 (m, 3H), 5.55-5.95 (m, 2H), 5.30 (m, 1H), 4.10-4.55 (m, 1H), 2.98 (m, 1H), 2.75-2.95 (m, 1H), 2.15 (m, 2H), 1.70-1.95 (m, 2H), 1.25 (m, 3H), 1.00 (m, 3H). MS: 508.1 (M−1), 532.2 (M+Na).

Compound 41:

N-[1(S)-benzoxazol-2-ylcarbonylpropyl]-2(S)-(4-acetylamino-3-methylphenylcarbonyl-amino)-4S-phenylpentamide. ¹H NMR (CDCl₃): δ 7.99 (d, J=7.6 Hz, 1H), 7.87 (d, J=6.0 Hz, 1H), 7.67 (d, J=7.6 Hz, 2H), 7.56 (m, 2H), 7.39-7.50 (m, 3H), 7.24-7.33 (m, 2H), 7.19 (m, 1H), 7.09 (m, 1H), 6.70 (d, J=6.4 Hz, 1H), 6.54 (d, J=7.6 Hz, 1H), 5.57 (m, 1H), 4.51 (m, 1H), 2.92 (m, 1H), 2.27 (s, 3H), 2.23 (m, 3H), 2.08-2.14 (m, 1H), 2.05 (m, 2H), 1.87 (m, 1H), 1.27 (m, 3H), 0.95 (m, 3H). MS: 553.4 (M−1), 555.5 (M+1), 577.4 (M+Na).

Compound 42:

N-[1(s)-2-phenyl[1,3,4]oxadiazol-5-ylcarbonylpropyl]-2(S)-(2′-chlorobiphen-4-ylcarbonylamino)-2-cyclohexylacetamide. H¹ NMR (DMSO-d₆): δ 8.75 (d, 1H), 8.39 (d, 1H), 8.1 (d, 2H), 7.9 (d, 2H), 27.7-7.5 (m, 4H), 7.5-7.3 (m, 5H), 5.1-4.9 (m, 1H), 4.5-4.3 (t, 1H), 2.1-1.95 (m, 1H), 1.9-1.5 (m, 8H]), 1.1-1 (m, 4H), 0.99 (t, 3H). MS: 585.3 (M+1), 607.4 (M+Na).

Compound 43:

N-[1(S)-benzoxazol-2-ylcarbonylpropyl]-2(S)-(2′-chlorobiphen-4-ylcarbonylamino)-3,indol-3-ylpropionamide. ¹H-NMR (DMSO-d₆): δ 8.80 (m, 1H), 8.60 (m, 1H), 8.06˜7.0 (m, 17H), 5.28 (m, 1H), 4.86 (m, 1H), 3.40˜3.05 (m, 2H), 2.08˜1.68 (m, 2H), 1.06˜0.86 (m, 3H). MS: 605 (M+1).

Compound 44:

N-[1 (S)-benzoxazol-2-ylcarbonylpropyl]-2(S)-(2′-chlorobiphen-4-ylcarbonylamino)-3-(N-phenyl-N-methylamino)propionamide. ¹H NMR (DMSO-d₆): 67.8-7.7 (m, 3H), 7.6 (d, 1H), 7.55-7.45 (m, 1H), 7.45-7.35 (m, 5H), 7.32 (d, 1H), 7.28-7.2 (m, 3H), 7.15-7.05 (m, 3H), 6.85 (d, 1H), 6.57 (t, 1H), 5.5-5.4 (m, 1H), 4.92-4.8 (m, 1H), 3.83 (d,d, 1H), 3.39 (d,d, 1H), 2.96 (s, 3H), 2.1-2 (m, 1H), 1.9-1.79 (m, 1H), 0.88 (t, 3H). MS: 593.3(M−1), 595.5 (M+1).

Compound 45:

N-[1-(S)-2-phenyl[1,3,4]oxadiazol-5-ylcarbonylpropyl]-2(S)-(2′,3-dichlorobiphen-4-ylcarbonylamino)-2-cyclohexylacetamide. ¹H NMR (DMSO-d₆): δ 8.8 (d, 1H), 8.45 (d, 1H), 8.05-7.9 (m, 3H), 7.8 (d, 1H), 7.7-7.2 (m, 8H), 5.1-4.9(m, 1H), 4.4 (t, 1H), 2.1-1.9 (m, 1H), 1.8-1.4 (m, 7H), 1.3-0.8 (m, 8H). MS: 641.3 (M+Na).

Compound 46:

N-[1(s)-2-tert-butyl[1,3,4]oxadiazol-5-ylcarbonylbutyl]-2(RS)-(2′-chlorobiphen-4-ylcarbonylamino)-3-(2,6-difluorophenyl)propionamide. ¹H-NMR (DMSO-d₆): δ 8.76˜8.54 (m, 2H), 7.92˜6.94 (m, 11H), 5.04 (m, 1H), 4.82 (m, 1H), 3.28˜2.98 (m, 2H), 1.90˜1.54 (m, 2H), 1.48˜1.14 (m, 9H), 0.94˜0.82 (m, 3H). MS: 609 (M+1).

Compound 47:

N-[1(S)-benzoxazol-2-ylcarbonylpropyl]-2(S)-[3,5-di(2-methoxyphenyl)phenylcarbonyl-amino]propionamide. ¹H NMR (DMSO-d₆): δ 8.58 (s, 2H), 8.00˜7.04 (m, 15H), 5.21 (m, 1H), 4.60 (m, 1H), 3.76 (s, 6H), 2.02 (m, 1H), 1.76 (m, 1H), 0.89 (d, J=20 Hz, 6H). MS: 592 (M+1).

Compound 48:

N-[1 (S)-benzoxazol-2-ylcarbonylpropyl]-2(RS)-(2′-chlorobiphen-4-ylcarbonylamino)-3-(4-methylindol-3-yl)propionamide. ¹H-NMR (DMSO-d₆): δ 8.64˜8.72 (m, 2H), 8.10˜6.68 (m, 14H), 5.24 (m, 1H), 4.82 (m, 1H), 2.62 (m, 2H), 2.18˜1.70 (m, 3H), 1.30˜0.76 (m, 5H). MS: 619 (M+1).

Compound 49:

N-[1(S)-benzoxazol-2-ylcarbonylpropyl]-2(S)-(5′-carboxy-2′-chlorobiphen-4-ylcarbonylamino)propionamide. ¹H-NMR (DMSO-d₆): δ 8.64˜8.52 (m, 2H), 8.00˜7.8 (m, 6H), 7.74˜7.50 (m, 5H), 5.23 (m, 1H), 4.60 (m, 1H), 2.01 (m, 1H), 1.76 (m, 1H), 1.35 (t, 3H), 0.99 (m, 3H). MS: 534 (M+1).

Compound 50:

N-[1(RS)-2-phenyl[1,3,4]oxadiazol-5-ylcarbonylpropyl]-2(R)-(2′-chlorobiphen-4-ylcarbonylamino)-4S-phenylpentamide. ¹H NMR (DMSO-d₆): δ 8.65-8.5 (m, 2H), 8.15-8.02 (m, 2H), 7.95-7.83 (m, 2H), 7.72-7.52 (m, 4H), 7.52-7.35 (m, 5H), 7.3-7.16 (m, 3H), 7.16-7.08 (m, 2H), 5.07-4.949 (m, 1H), 4.17 (m, 1H), 2.85-2.79 (m, 1H), 2.0-1.8 (m, 3H), 1.8-1.65 (m, 1H), 1.25-1.1 (d, 3H), 0.92 (t, 3H). MS: 519.6(M−1), 621.3 (M+1), 643.5 (M+Na).

Compound 51:

N-[1(S)-benzoxazol-2-ylcarbonylpropyl]-2(S)-(2′,3-dichlorobiphen-4-ylcarbonylamino)-propionamide. ¹H NMR (DMSO-d₆): δ 8.70 (m, 1H), 8.61 (m, 1H), 8.10-7.30 (m, 10H), 5.20 (m, 1H), 4.59 (m, 1H), 2.01 (m, 1H), 1.76 (m, 1H), 1.35 (t, 3H), 1.01 (m, 3H). MS: 524 (M+1).

Compound 54:

2′-Chlorobiphen-4-ylcarboxylic acid {1-[1(S)-oxazolo[4,5-b]pyridin-2-ylcarbonyl)-propylaminocarbonyl]cycloheptyl}amide; NMR: δ 9.2 (s, 1H), 8.78 (s, 1H), 8.45 (d, J=6.8 Hz, 1H), 8.20 (s, 1H), 7.9 (d, J=8.0 Hz, 2H), 7.40-7.70 (m, 8H), 5.38 (s, 1H), 1.05-2.2 (m, 17H). MS: M−1 (558.6).

Compound 55:

2′,3-Dichlorobiphen-4-ylcarboxylic acid {1-[1(S)-oxazolo[4,5-b]pyridin-2-ylcarbonyl)-propylaminocarbonyl]cycloheptyl}amide MS: M−1 (592.1).

Compound 56:

2′,3-Dichlorobiphen-4-ylcarboxylic acid {1-[1-benzoxazol-2-ylcarbonyl)propyl-aminocarbonyl]cycloheptyl}amide MS: M−2 (590.1).

### BIOLOGICAL EXAMPLES

### Example 1

### Cathepsin B Assay

Solutions of test compounds in varying concentrations were prepared in 10 μL of dimethyl sulfoxide (DMSO) and then diluted into assay buffer (40 μL, comprising: N,N-bis(2-hydroxyethyl)-2-aminoethanesulfonic acid (BES), 50 mM (pH 6); polyoxyethylenesorbitan monolaurate, 0.05%; and dithiothreitol (DTT), 2.5 mM). Human cathepsin B (0.025 pMoles in 25 μL of assay buffer) was added to the dilutions. The assay solutions were mixed for 5-10 seconds on a shaker plate, covered and incubated for 30 minutes at room temperature. Z-FR-AMC (20 nMoles in 25 μL of assay buffer) was added to the assay solutions and hydrolysis was followed spectrophotometrically at (λ460 nm) for 5 minutes. Apparent inhibition constants (Kᵢ) were calculated from the enzyme progress curves using standard mathematical models.

Compounds of the invention were tested by the above-described assay and observed to exhibit cathepsin B inhibitory activity.

### Example 2

### Cathepsin K Assay

Solutions of test compounds in varying concentrations were prepared in 10 μL of dimethyl sulfoxide (DMSO) and then diluted into assay buffer (40 μL, comprising: MES, 50 mM (pH 5.5); EDTA, 2.5 mM; and DTT, 2.5 mM). Human cathepsin K (0.0906 pMoles in 25 μL of assay buffer) was added to the dilutions. The assay solutions were mixed for 5-10 seconds on a shaker plate, covered and incubated for 30 minutes at room temperature. Z-Phe-Arg-AMC (4 nMoles in 25 μL of assay buffer) was added to the assay solutions and hydrolysis was followed spectrophotometrically at (λ 460 nm) for 5 minutes. Apparent inhibition constants (Kᵢ) were calculated from the enzyme progress curves using standard mathematical models.

Compounds of the invention were tested by the above-described assay and observed to exhibit cathepsin K inhibitory activity.

### Example 3

### Cathepsin L Assay

Solutions of test compounds in varying concentrations were prepared in 10 μL of dimethyl sulfoxide (DMSO) and then diluted into assay buffer (40 μL, comprising: MES, 50 mM (pH 5.5); EDTA, 2.5 mM; and DTT, 2.5 mM). Human cathepsin L (0.05 pMoles in 25 μL of assay buffer) was added to the dilutions. The assay solutions were mixed for 5-10 seconds on a shaker plate, covered and incubated for 30 minutes at room temperature. Z-Phe-Arg-AMC (1 nMoles in 25 μL of assay buffer) was added to the assay solutions and hydrolysis was followed spectrophotometrically at (λ 460 nm) for 5 minutes. Apparent inhibition constants (Kᵢ) were calculated from the enzyme progress curves using standard mathematical models.

Compounds of the invention were tested by the above-described assay and observed to exhibit cathepsin L inhibitory activity.

### Example 4

### Cathepsin S Assay

Solutions of test compounds in varying concentrations were prepared in 10 μL of dimethyl sulfoxide (DMSO) and then diluted into assay buffer (40 μL, comprising: MES, 50 mM (pH 6.5); EDTA, 2.5 mM; and NaCl, 100 mM); p-mercaptoethanol, 2.5 mM; and BSA, 0.00%. Human cathepsin S (0.05 pMoles in 25 μL of assay buffer) was added to the dilutions. The assay solutions were mixed for 5-10 seconds on a shaker plate, covered and incubated for 30 minutes at room temperature. Z-Val-Val-Arg-AMC (4 nMoles in 25 μL of assay buffer containing 10% DMSO) was added to the assay solutions and hydrolysis was followed spectrophotometrically (at λ 460 nm) for 5 minutes. Apparent inhibition constants (Kᵢ) were calculated from the enzyme progress curves using standard mathematical models.

Compounds of the invention were tested by the above-described assay and observed to exhibit cathepsin S inhibitory activity.

### Example 5

### Cathepsin F Assay

Solutions of test compounds in varying concentrations were prepared in 10 μL of dimethyl sulfoxide (DMSO) and then diluted into assay buffer (40 μL, comprising: MES, 50 mM (pH 6.5); EDTA, 2.5 mM; and NaCl, 100 mM); DIT, 2.5 mM; and BSA, 0.01%. Human cathepsin F (0.1 pMoles in 25 μL of assay buffer) was added to the dilutions. The assay solutions were mixed for 5-10 seconds on a shaker plate, covered and incubated for 30 minutes at room temperature. Z-Phe-Arg-AMC (2 nMoles in 25 μL of assay buffer containing 10% DMSO) was added to the assay solutions and hydrolysis was followed spectrophotometrically (at λ 460 nm) for 5 minutes. Apparent inhibition constants (Kᵢ) were calculated from the enzyme progress curves using standard mathematical models.

Compounds of the invention were tested by the above-described assay and observed to exhibit cathepsin F inhibitory activity.

### Pharmaceutical Composition Examples

The following are representative pharmaceutical formulations containing a compound of Formula I.

### Tablet Formulation

The following ingredients are mixed intimately and pressed into single scored tablets.

| | Quantity per |
|---|---|
| Ingredient | tablet, mg |
| compound of this invention | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| | Quantity per |
|---|---|
| Ingredient | capsule, mg |
| compound of this invention | 200 |
| lactose, spray-dried | 148 |
| magnesium stearate | 2 |

### Suspension Formulation

The following ingredients are mixed to form a suspension for oral administration.

| Ingredient | Amount | |
|---|---|---|
| compound of this invention | 1.0 | g |
| fumaric acid | 0.5 | g |
| sodium chloride | 2.0 | g |
| methyl paraben | 0.15 | g |
| propyl paraben | 0.05 | g |
| granulated sugar | 25.5 | g |
| sorbitol (70% solution) | 12.85 | g |
| Veegum K (Vanderbilt Co.) | 1.0 | g |
| flavoring | 0.035 | mL |
| colorings | 0.5 | mg |
| distilled water | q.s. to 100 | mL |

### Injectable Formulation

The following ingredients are mixed to form an injectable formulation.

| Ingredient | Amount |
|---|---|
| compound of this invention | 1.2 g |
| sodium acetate buffer solution, | 0.4 M 2.0 mL |
| HCl (1 N) or NaOH (1 N) | q.s. to suitable pH |
| water (distilled, sterile) | q.s.to 20 mL |

All of the above ingredients, except water, are combined and heated to 60-70° C. with stirring. A sufficient quantity of water at 60° C. is then added with vigorous stirring to emulsify the ingredients, and water then added q.s. to 100 g.

### Suppository Formulation

A suppository of total weight 2.5 g is prepared by mixing the compound of the invention with Witepsol.RTM. H-15 (triglycerides of saturated vegetable fatty acid; Riches-Nelson, Inc., New York), and has the following composition:

| | |
|---|---|
| compound of the invention | 500 mg |
| Witepsol ® H-15 | balance |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

1. A compound of Formula I: 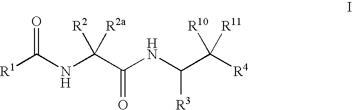
wherein:
R¹ is a group of formula: 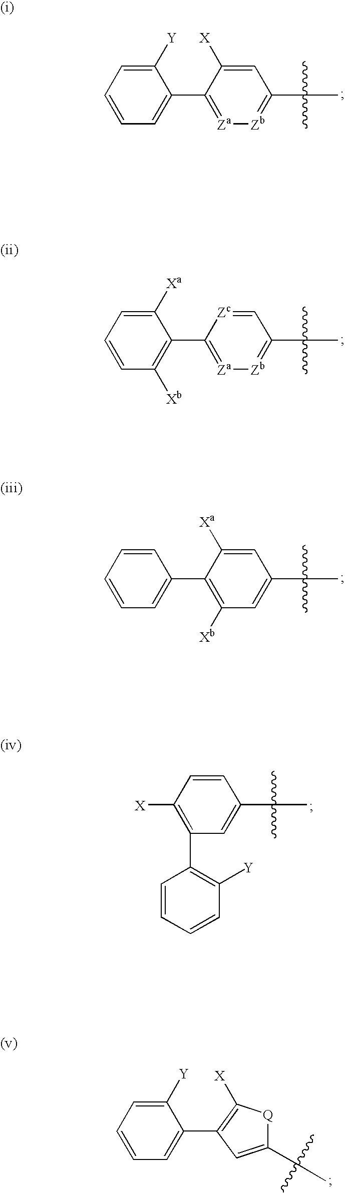 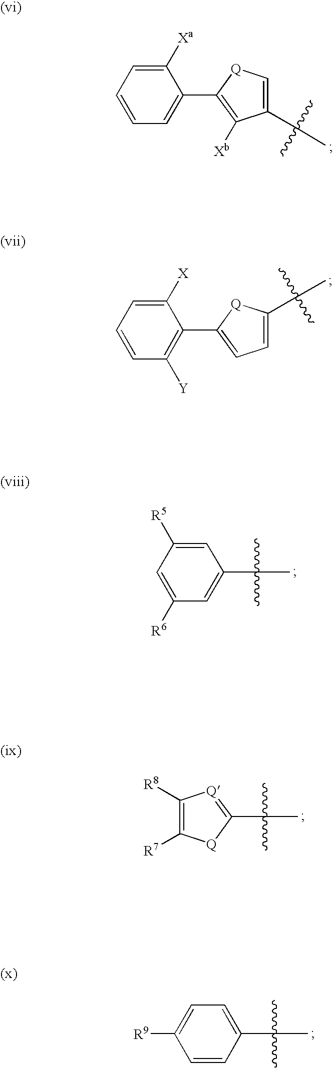 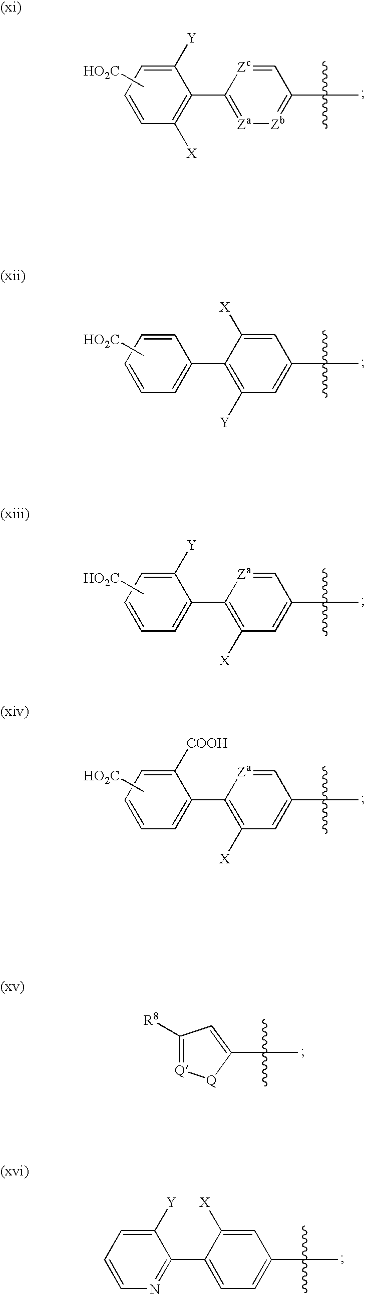
(xvii) 1-(4-aminosulfonylphenyl)-5-(4-chlorophenyl)pyrazol-3-yl;
(xviii) 1-methyl-3-trifluoro-1H-thieno-[2,3-c]-pyrazol-5-yl;
(xix) 4-(3,5-dimethyloxazol-4-yl)phenyl;
(xx) 4-(5-carboxy-2-methylthiophen-3-yl)phenyl;
(xxi) 3-vinylphenyl;
(xxii) 4-phenoxyphenyl;
(xxiii) 4-acetylamino-3-methylphenyl; or
(xxiv) 4-morpholin-4-ylphenyl;
where:
Z^{a} is —CX— or —N— and Z^{b} and Z^{c} are independently selected from —H— and —N— provided that if an R¹ group contains Z^{a}, Z^{b}, and Z^{c} simultaneously then, when Z^{c} is —N—, then Z^{a} is —N— or —CX— and Z^{b} is —CH—; and when Z^{b} is —N— then both Z^{a} and Z^{c} cannot be —N— simultaneously;
Q is —NR— where R is hydrogen or alkyl, —O—, or —S—;
Q′ is —CH— or —N—;
X and Y are independently selected from hydrogen, halo, alkyl, alkoxy, haloalkyl, or haloalkoxy provided that both X and Y are not simultaneously hydrogen;
X^{a} and X^{b} are independently selected from alkyl, halo, alkoxy, haloalkyl, or haloalkoxy;
R⁵ and R⁶ are independently selected from phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl;
R⁷ and R⁸ are independently selected from phenyl, 2-alkoxyphenyl, 3-alkoxyphenyl, 2′-halophenyl, 2-alkylphenyl, 2-haloalkylphenyl, 2-haloalkoxyphenyl, furan-2-yl, thiophen-3-yl, or pyridin-4-yl; and
R⁹ is a branched alkyl chain of 4-6 carbon atoms or trifluoroalkoxy;
R² is selected from the group consisting of hydrogen, methyl, ethyl, n-propyl, 2-propyl, cyclopentyl, cyclohexyl, cycloheptyl, 2-ethylbutyl, thiazolylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), benzyl (where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino), heteroaryl(C₃₋₆)alkyl and 1-heteroaryl(C₃₋₆)cycloalkylmethyl and furthermore wherein the alkyl chain in the above groups is optionally substituted with one to six halo;
R^{2a} is hydrogen or R^{2a} and R² together with the carbon atom to which they are attached form cyclohexyl or cycloheptyl;
R³ is ethyl, propyl, or n-butyl;
R⁴ is benzoxazol-2-yl, oxazolo[4,5-b]pyridin-2-yl, 2-pyridin-3-yl-[1,3,5]-oxadiazol-5-yl, 2-pyridin-4-yl-[1,3,4]-oxadiazol-5-yl, 2-ethyl-[1,3,4]-oxadiazol-5-yl, 2-phenyl-[1,3,4]-oxadiazol-5-yl, pyrazin-2-yl, pyrimidin-2-yl, pyridazin-3-yl, 3-phenyl-[1,2,4]-oxadiazol-5-yl, or 3-ethyl-[1,2,4]-oxadiazol-5-yl;
R¹⁰ is hydrogen, hydroxy, alkoxy; and
R¹¹ is hydroxy or alkoxy; or
R¹⁰ and R¹¹ together with the carbon atom to which they are attached form (═O) or —O—(C₂-C₄)alkylene-O— wherein the alkylene chain is optionally substituted with one or two alkyl; or a pharmaceutically acceptable salt thereof.

2. A compound of Formula Ia: 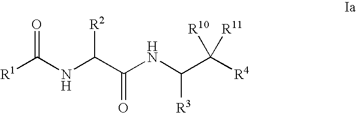
wherein:
R¹ is a group of formula: 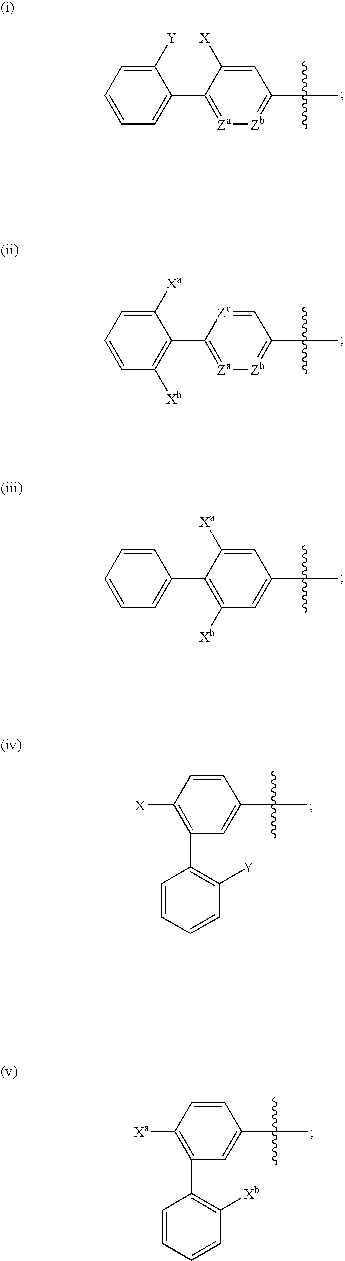 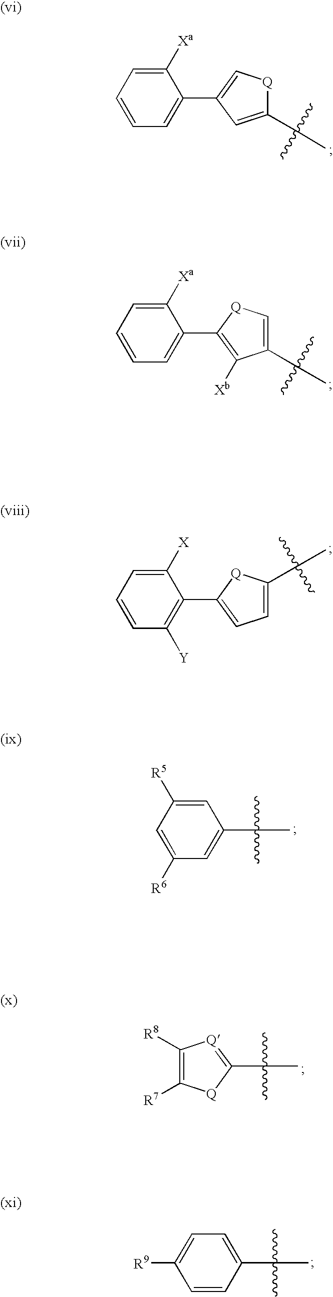
(xii) 1-(4-aminosulfonylphenyl)-5-(4-chlorophenyl)pyrazol-3-yl;
(xiii) 1-methyl-3-trifluoro-1H-thieno-[2,3-c]-pyrazol-5-yl; 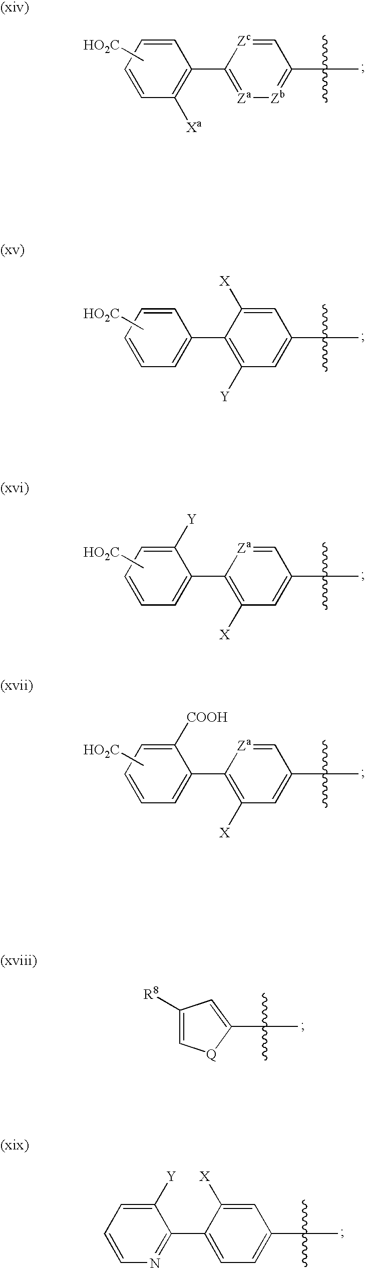
(xx) 4-(3,5-dimethyloxazol-4-yl)phenyl; or
(xxi) 4-(5-carboxy-2-methylthiophen-3-yl)phenyl;
where:
Z^{a}, Z^{b}, and Z^{c} are independently selected from —CH— or —N— provided that if an R¹ group contains Z^{a}, Z^{b}, and Z^{c} simultaneously then, when Z^{c} is —N—, then Z^{a} is —N— or —CH— and Z^{b} is —H—; and when Z^{b} is —N— then Z^{a} and Z^{c} are —CH—; and if an R¹ group contains Z^{a} and Z^{b} simultaneously, then both Z^{a} and Z^{b} cannot simultaneously be —N—;
Q is —NR— where R is hydrogen or alkyl, —O—, or —S—;
Q′ is —H— or —N—;
X and Y are independently selected from hydrogen, halo, alkyl, alkoxy, haloalkyl, or haloalkoxy provided that both X and Y are not simultaneously hydrogen;
X^{a} and X^{b} are independently selected from alkyl, halo, alkoxy, haloalkyl, or haloalkoxy;
R⁵ and R⁶ are independently selected from phenyl, 2-alkoxyphenyl, 3-alkoxyphenyl, 2-halophenyl, 2-alkylphenyl, 2-haloalkoxyphenyl, furan-2-yl, thiophen-3-yl, or pyridin-4-yl;
R⁷ and R⁸ are independently selected from phenyl, 2-alkoxyphenyl, 3-alkoxyphenyl, 2-halophenyl, 2-alkylphenyl, 2-haloalkoxyphenyl, furan-2-yl, thiophen-3-yl, or pyridin-4-yl; and
R⁹ is a branched alkyl chain of 4-6 carbon atoms or trifluoroalkoxy;
R² is selected from the group consisting of cyclopentyl, cyclohexyl, cycloheptyl, 2-ethylbutyl, thiazolylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl wherein the phenyl group in 1-phenyl-cyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy, and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino;
R³ is ethyl, propyl, or n-butyl; and
R⁴ is benzoxazol-2-yl, oxazolo[4,5-b]pyridin-2-yl, 2-pyridin-3-yl-[1,3,5]-oxadiazol-5-yl, 2-pyridin-4-yl-[1,3,4]-oxadiazol-5-yl, 2-ethyl-[1,3,4]-oxadiazol-5-yl, 2-phenyl-[1,3,4]-oxadiazol-5-yl, pyrazin-2-yl, pyrimidin-2-yl, pyridazin-3-yl, 3-phenyl-[1,2,4]-oxadiazol-5-yl, or 3-ethyl-[1,2,4]-oxadiazol-5-yl;
R¹⁰ is hydrogen, hydroxy, alkoxy; and
R¹¹ is hydroxy or alkoxy; or
R¹⁰ and R¹¹ together with the carbon atom to which they are attached form (═O) or —O—(C₂-C₄)alkylene-O wherein the alkylene chain is optionally substituted with one or two alkyl; or a pharmaceutically acceptable salt thereof.

3. The compound of claim 2 wherein:
R¹ is a group of formula: 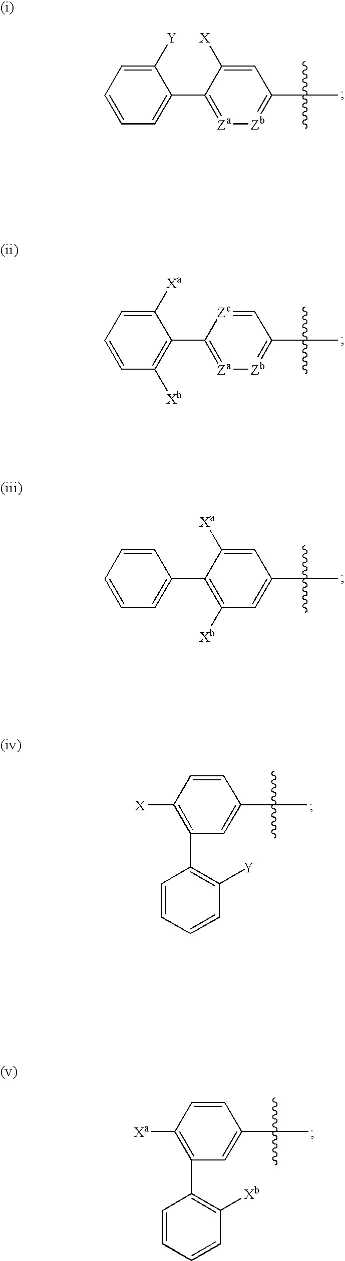 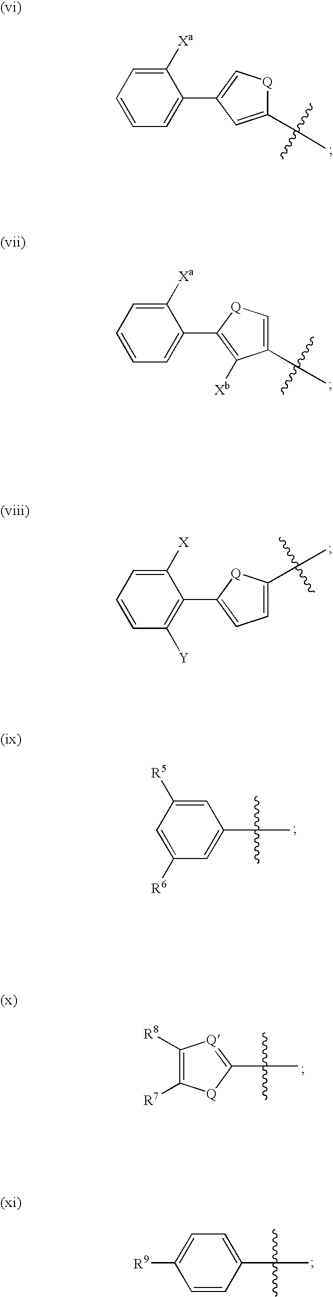
(xii) 1-(4-aminosulfonylphenyl)-5-(4-chlorophenyl)pyrazol-3-yl; or
(xiii) 1-methyl-3-trifluoro-1H-thieno-[2,3-]-pyrazol-4-yl;
wherein:
Z^{a}, Z^{b} and Z^{c} are —CH—;
X and Y are independently selected from hydrogen, chloro, methyl, methoxy, trifluoromethyl, or trifluoromethoxy;
X^{a}, and X^{b} are independently selected from methyl, chloro, fluoro, methoxy, trifluoromethyl, or trifluoromethoxy; and
R¹⁰ and R¹¹ together with the carbon atom to which they are attached form (═O).

4. The compound of claim 2 or 3 wherein:
X and Y are independently selected from hydrogen, chloro, methyl, methoxy, or trifluoromethoxy;
X^{a}, and X^{b} are independently selected methyl, chloro, fluoro, methoxy, or trifluoromethoxy;
R² is selected from the group consisting of 2-methylpropyl, 2,4,4-trimethylpentyl, 2-napth-1-ylpropyl, 2-phenylprop-2-enyl, 2-phenyl-2-methylpropyl, 2-phenylpropyl, 2-(2-methoxy-phenyl)propyl, 4-methylindol-3-ylmethyl, 2-(2,5-dimethylphenyl)propyl, benzyloxymethyl, 2-(2,4-dimethylphenyl)propyl, 2-(2,4-dichlorophenyl)propyl, 2,6-difluorobenzyl, 2,5-difluorobenzyl, and 2,3-difluorobenzyl; and the stereochemistry at the carbon to which R² is attached is (S); and
R¹⁰ and R¹¹ together with the carbon atom to which they are attached form (═O).

5. The compound of claim 1, 2, 3, or 4 wherein R¹ is 2′-chlorobiphen-4-yl, 2′,3-dichlorobiphenyl-4-yl, 2′,6′-dichlorobiphen-4-yl, 2′,6′-dimethylbiphen-4-yl, 2′methylbiphen-4-yl, 2′-fluorobiphen-4-yl; 4-trifluoromethoxyphenyl, 4-2-butyl)phenyl, 3,5-diphenylphenyl, 2,3-diphenylthiophen-5-yl, 2-(2-methylphenyl)furan-5-yl, 2-(2-methoxyphenyl)furan-5-yl, 3-methoxy-2-(2-methylphenyl)thiophen-4-yl, 3-methoxy-2-(2-methoxyphenyl)thiophen-4-yl, 2,3-di(2-methoxyphenyl)thiophen-5-yl, 3,5-di(2-methoxyphenyl)phenyl, 3,5-di(3-methoxyphenyl)-phenyl, 3,5-di(thiophen-3-yl)phenyl, 3,5-di(pyridin-4-yl)phenyl, 2,3-di(2-methylphenyl)-thiophen-5-yl, 4-tert-butylphenyl, 2,3-di(furan-2-yl)thiophen-5-yl, 3,5-di(furan-2-yl)phenyl, 4-(2-methylphenyl)thiophen-2-yl, 4-(2-methoxyphenyl)thiophen-2-yl, 2′-chlorobiphen-3-yl, 2′-methyl-4-chlorobiphenyl-3-yl, 3,5-di(2-chlorophenyl)phenyl, 2,3-di(2-chlorophenyl)thiophen-5-yl, 1-(4-aminosulfonylphenyl)-5-(4-chlorophenyl)pyrazol-3-yl, 2-(2,6-dichlorophenyl)furan-5-yl, 2,3-diphenylthiophen-5-yl, 4,5-diphenylthiazol-2-yl, 3-trifluoromethyl-1-methylthieno[2,3-c]pyrazol-5-yl, 3-methylbiphen-4-yl, 2′-methoxybiphen-4-yl, 2′-trifluoromethylbiphen-4-yl, or 2′-methyl-3-chlorobiphen-4-yl.

6. The compound of claim 1, 2, 3, or 4 wherein R¹ is 2′-chlorobiphen-4-yl, 2′,3-dichlorobiphenyl-4-yl, 2′,6′-dichlorobiphen-4-yl, 2′,6′-dimethylbiphen-4-yl, 2′-methylbiphen-4-yl, 2′-fluorobiphen-4-yl; 4-trifluoromethoxyphenyl, 4-(2-butyl)phenyl, 3,5-diphenylphenyl, 2,3-diphenylthiophen-5-yl, 2-(2-methylphenyl)furan-5-yl, 2-(2-methoxyphenyl)furan-5-yl, 3-methoxy-2-(2-methylphenyl)thiophen-4-yl, 3-methoxy-2-(2-methoxyphenyl)thiophen-4-yl, 2,3-di(2-methoxyphenyl)thiophen-5-yl, 3,5-di(2-methoxyphenyl)phenyl, 3,5-di(3-methoxyphenyl)-phenyl, 3,5-di(thiophen-3-yl)phenyl, 3,5-di(pyridin-4-yl)phenyl, 2,3-di(2-methylphenyl)-thiophen-5-yl, 4-tert-butylphenyl, 2,3-di(furan-2-yl)thiophen-5-yl, 3,5-di(furan-2-yl)phenyl, 4-(2-methylphenyl)thiophen-2-yl, 4-(2-methoxyphenyl)thiophen-2-yl, 2′-chlorobiphen-3-yl, 2′-methyl-4-chlorobiphenyl-3-yl, 3,5-di(2-chlorophenyl)phenyl, 2,3-di(2-chlorophenyl)thiophen-5-yl, 1-(4-aminosulfonylphenyl)-5-(4-chlorophenyl)pyrazol-3-yl, 2-(2,6-dichlorophenyl)furan-5-yl, 2,3-diphenylthiophen-5-yl, 4,5-diphenylthiazol-2-yl, or 3-trifluoromethyl-1-methylthieno-[2,3-c]-pyrazol-5-yl.

7. The compound of claim 1, 2, 3, 4, 5, or 6 wherein R³ is ethyl and the stereochemistry at the carbon atom to which R³ is attached is (S).

8. The compound of any of the claim 1-7 wherein R⁴ is benzoxazol-2-yl.

9. The compound of claim 2 wherein:
R¹ is a group a group of formula (i), (ii) and (xiv)-(xxi) wherein:
Z^{a}, Z^{b}, and Z^{c} are independently selected from —CH— or —N— provided that when R¹ is a group of formula (i) then one of Z^{a} and Z^{b} is —N— and the other is —CH—; when R¹ is a group of formula (ii), then Z^{c} is —N—, Z^{a} is —N— or —CH— and Z^{b} is —H—; or Z^{b} is —N— and Z^{a} and Z^{c} are —CH—; and when R¹ is a group of formula (xiv), then when Z^{c} is —N—, then Z^{a} is —N— or —CH—, and Z^{b} is —H—; and when Z^{b} is —N—, then Z^{a} and Z^{c} are —CH—;
X and Y are independently selected from hydrogen, chloro, methyl, methoxy, trifluoromethyl, or trifluoromethoxy;
X^{a}, and X^{b} are independently selected from methyl, chloro, fluoro, methoxy, trifluoromethyl, or trifluoromethoxy; and
R¹⁰ and R¹¹ together with the carbon atom to which they are attached form (═O).

10. The compound of claim 2 wherein:
R¹ is a group a group of formula (i), (ii) and (xiv)-(xxi) wherein:
Z^{a}, Z^{b}, and Z^{c} are independently selected from —CH— or —N— provided that when R¹ is a group of formula (i) then one of Z^{a} and Z^{b} is —N— and the other is —CH—; when R¹ is a group of formula (ii), then Z^{c} is —N—, Z^{a} is —N— or —CH— and Z^{b} is —H—; or Z^{b} is —N— and Z^{a} and Z^{c} are —CH—; and when R¹ is a group of formula (xiv), then when Z^{b} is —N—, then Z^{a} is —N— or —CH—, and Z^{b} is —CH—; and when Z^{b} is —N—, then Z^{a} and Z^{c} are —CH—;
X and Y are independently selected from hydrogen, chloro, methyl, methoxy, trifuoromethyl, or trifluoromethoxy;
X^{a}, and X^{b} are independently selected from methyl, chloro, fluoro, methoxy, trifluoromethyl, or trifluoromethoxy;
R² is selected from the group consisting of 2-methylpropyl, 2,4,4-trimethylpentyl, 2-napth-1-ylpropyl, 2-phenylprop-2-enyl, 2-phenyl-2-methylpropyl, 2-phenylpropyl, 2-(2-methoxy-phenyl)propyl, 4-methylindol-3-ylmethyl, 2-(2,5-dimethylphenyl)propyl, benzyloxymethyl, 2-(2,4-dimethylphenyl)propyl, 2-(2,4-dichlorophenyl)propyl, 2,6-difluorobenzyl, 2,5-difluorobenzyl, and 2,3-difluorobenzyl; and the stereochemnistry at the carbon to which R² is attached is (S); and
R¹⁰ and R¹¹ together with the carbon atom to which they are attached form (═O).

11. The compound of any of the claims 1, 2, 9 and 10 wherein R¹ is 2-(2-chloro-phenyl)pyridin-5-yl, 2-(2′,6′-dichlorophenyl)pyridin-5-yl, 2-(2-trifluoromethylphenyl)pyridin-5-yl, 4-(3-methylpyridin-2-yl)phenyl, 2-(2-chlorophenyl)-3-chloropyridin-5-yl, 4′-carboxy-2′-chlorobiphen-4-yl, 4′-carboxy-2′-fluorobiphen-4-yl, 4′-carboxy-2′-methylbiphen-4-yl, 5′-carboxy-2′-chlorobiphen-4-yl, 2-(4-carboxy-2-chlorophenyl)pyridin-5-yl, 2-(5-carboxy-2-chlorophenyl)pyridin-5-yl, 4-(5-carboxy-2-methylthiophen-3-yl)phenyl, or 4-(3-methoxy-phenyl)thiophen-2-yl.

12. The compound of claim 11 wherein R³ is ethyl and the stereochemistry at the carbon atom to which R³ is attached is (S).

13. The compound of claim 12 wherein R⁴ is benzoxazol-2-yl.

14. The compound of claim 1 or 2 wherein R¹ is 2′-chlorobiphen-4-yl, 2′,3-dichloro-biphenyl-4-yl, 2′,6′-dichlorobiphen-4-yl, 2′,6′-dimethylbiphen-4-yl, 2′-methylbiphen-4-yl, 2′-fluorobiphen-4-yl; 4-trifluoromethoxyphenyl, 4-(2-butyl)phenyl, 3,5-diphenylphenyl, 2,3-diphenylthiophen-5-yl, 2-(2-methylphenyl)furan-5-yl, 2-(2-methoxyphenyl)furan-5-yl, 3-methoxy-2-(2-methylphenyl)thiophen-4-yl, 3-methoxy-2-(2-methoxyphenyl)thiophen-4-yl, 2,3-di(2-methoxyphenyl)thiophen-5-yl, 3,5-di(2-methoxyphenyl)phenyl, 3,5-di(3-methoxyphenyl)-phenyl, 3,5-di(thiophen-3-yl)phenyl, 3,5-di(pyridin-4-yl)phenyl, 2,3-di(2-methylphenyl)-thiophen-5-yl, 4-tert-butylphenyl, 2,3-di(furan-2-yl)thiophen-5-yl, 3,5-di(furan-2-yl)phenyl, 4-(2-methylphenyl)thiophen-2-yl, 4-(2-methoxyphenyl)thiophen-2-yl, 2′-chlorobiphen-3-yl, 2′-methyl-4-chlorobiphenyl-3-yl, 3,5-di(2-chlorophenyl)phenyl, 2,3-di(2-chlorophenyl)thiophen-5-yl, 1-(4-aminosulfonylphenyl)-5-(4-chlorophenyl)pyrazol-3-yl, 2-(2,6-dichlorophenyl)furan-5-yl, 2,3-diphenylthiophen-5-yl, 4,5-diphenylthiazol-2-yl, 3-trifluoromethyl-1-methylthieno[2,3-c]pyrazol-5-yl, 3-methylbiphen-4-yl, 2′-methoxybiphen-4-yl, 2′-trifluoromethylbiphen-4-yl, 2′-methyl-3-chlorobiphen-4-yl, 2-(2-chlorophenyl)pyridin-5-yl, 2-(2,6-dichlorophenyl)pyridin-5-yl, 2-(2-trifluoromethylphenyl)pyridin-5-yl, 4-(3-methylpyridin-2-yl)phenyl, 2-(2-chloro-phenyl)-3-chloropyridin-5-yl, 4′-carboxy-2′-chlorobiphen-4-yl, 4′-carboxy-2′-fluorobiphen-4-yl, 4′-carboxy-2′-methylbiphen-4-yl, 5′-carboxy-2′-methylbiphen-4-yl, 4-(3-methylpyridin-2-yl)phenyl, 5′-carboxy-2′-chlorobiphen-4-yl, 2-(4-carboxy-2-chlorophenyl)pyridin-5-yl, 2-(5-carboxy-2-chlorophenyl)pyridin-5-yl, 4-(5-carboxy-2-methylthiophen-3-yl)phenyl, 3-chloro-2-(2,6-dichlorophenyl)pyridin-5-yl, 3-(2-chlorophenyl)isoxazol-5-yl or 4-(3-methoxy-phenyl)thiophen-2-yl;
R² is selected from the group consisting of hydrogen, methyl, ethyl, propyl, cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl, benzyl (where the phenyl ring in the benzyl group is substituted at the 2 and 6 positions with groups independently selected from methyl, chloro, fluoro, trifluoromethyl, methoxy, trifluoromethoxy, or difluoromethoxy and at the 4 position with hydrogen, methyl, ethyl, propyl, chloro, fluoro, trifluoromethyl, methoxy, 2-methoxyethyloxy, 2-dimethylaminoethyloxy, trifluoromethoxy, difluoromethoxy, methylthio, methylsulfinyl, methylsulfonyl, cyano, amino, methylamino or dimethylamino);
R^{2a} is hydrogen; and
R¹⁰ and R¹¹ together with the carbon atom to which they are attached form (═O).

15. The compound of claim 1 wherein:
R¹ is 2′-Cl-biphenyl-4-yl, 2,3-diphenylthiophen-5-yl, 2-(2-Clphenyl)pyridin-5-yl, 2′,3-diCl-biphen-4-yl, 5′-carboxy-2′chlorobiphen-4-yl, 3-vinylphenyl, 4-phenoxyphenyl, 4-acetylamino-3-methyl-phenyl, 3,5-di(2-methoxyphenyl)-phenyl, 4-morpholin-4-ylphenyl, 1-methyl-3-trifluoromethyl-1H-thieno[2,3-c]pyrazol-5-yl, or 4-tert-butylphenyl;
R² is 2,6-difluorobenzyl, 2(S)-phenylpropyl, cyclohexyl, thiazol-2-ylmethyl, cycloheptyl, 2-ethylbutyl, pyrazol-1-yl-methyl, 2,4,6-trifluorobenzyl, indol-3-ylmethyl, N-phenyl-N-methylaminomethyl, methyl, 4-methylindol-3-ylmethyl, or hydrogen;
R^{2a} is hydrogen or R² and R²′ together with the carbon atom to which they are attached form cycloheptyl,
R³ is ethyl, n-propyl, n-butyl;
R⁴ is benzoxazol-2-yl, oxazolo[4,5-b]pyridin-2-yl, 2-pyridin-3-yl-[1,3,4]-oxadiazol-5-yl, 2-pyridin-4-yl-[1,3,4]-oxadiazol-5-yl, 2-phenyl-[1,3,4]-oxadiazol-5-yl, 2-ethyl-[1,3,4]-oxadiazol-5-yl, 2-phenyl-[1,3,4]-oxadiazol-5-yl, or 2-tert-butyl-[1,3,4]-oxadiazol-5-yl; and
R¹⁰ and R¹¹ together with the carbon atom to which they are attached form —C═O.

16. A compound selected from:
N-[1(8)-benzoxazol-2-ylcarbonylpropyl]-2(5)-(2′-chlorobiphen-4-ylcarbonylamino)-3-(2,6-difluorophenyl)propionamide (compound 1);
N-[1(S)-benzoxazol-2-ylcarbonylpropyl]-2(RS)-(2′-chlorobiphen-4-ylcarbonylamino)-3-(2,6-difluorophenyl)propionamide (compound 2);
N-[1(S)-benzoxazol-2-ylcarbonylpropyl]-2(RS)-(2,3-diphenylthiophen-2-ylcarbonyl-amino)-3-(2,6-difluorophenyl)propionamide (compound 3);
N-[1(S)-benzoxazol-2-ylcarbonylpropyl]-2(S)-(2′-chlorobiphen-4-ylcarbonylamino)-4(s)-phenylpentamide (compound 4);
N-[1(RS)-benzoxazol-2-ylcarbonylbutyl]-2(RS)-(2′-chlorobiphen-4-ylcarbonylamino)-3-(2,6-difluorophenyl)propionamide(compound 5);
N-[1(S)-oxazolo-[4,5-b]-pyridin-2-ylcarbonylpentyl]-2(RS)-(2′-chlorobiphen-4-yl-carbonylamino)-3-(2,6-difluorophenyl)propionamide (compound 6);
N-[1 (RS)-benzoxazol-2-ylcarbonylpentyl]-2(RS)-(2′-chlorobiphen-4-ylcarbonylamino)-3-(2,6-difluorophenyl)propionamide(compound 7);
N-[1(S)-oxazolo-[4,5-b]-pyridin-2-ylcarbonylpropyl]-2(S)-(2′-chlorobiphen-4-yl-carbonylamino)-3-(2,6-difluorophenyl)propionamide (compound 8);
N-[1 (S)-oxazolo-[4,5-b]-pyridin-2-ylcarbonylpropyl]-2(RS)-(2′-chlorobiphen-4-yl-carbonylamino)-3-(2,6-difluorophenyl)propionamide (compound 9);
N-[1 (S)-(2-pyridin-3-yl-[1,3,4]-oxadiazol-5-ylcarbonyl)pentyl]-2(RS)-(2′-chlorobiphen-4-ylcarbonylamino)-3-(2,6-difluorophenyl)propionamide(compound 10);
N-[1(S)-(2-pyridin-4-yl-[1,3,4]-oxadiazol-5-ylcarbonyl)pentyl]-2(RS)-(2′-chlorobiphen-4-ylcarbonylamino)-3-(2,6-difluorophenyl)propionamide (compound 11);
N-[1 (S)-benzoxazol-2-ylcarbonylpropyl]-2(R)-(2′-chlorobiphen-4-ylcarbonylamino)-4(S)-phenylpentamide (compound 12);
N-[1(S)-(2-phenyl-[1,3,4]-oxadiazol-5-ylcarbonyl)pentyl]-2(RS)-(2′-chlorobiphen-4-yl-carbonylamino)-3-(2,6-difluorophenyl)propionamide (compound 13);
N-[1(S)-benzoxazol-2-ylcarbonylpropyl]-2(S)-[2-(2-chlorophenyl)pyridin-5-yl-carbonylamino]-3-(2,6-difluorophenyl)propionamide (compound 14);
N-[1(S)-oxazol-[4,5-b]-pyridin-2-ylcarbonylpropyl]-2(S)-(2′-chlorobiphen-4-yl-carbonylamino)-cyclohexylacetamide (compound 15);
N-[1(S)-(2-ethyl-[1,3,4]-oxadiazol-5-ylcarbonyl)butyl]-2(RS)-(2′-chlorobiphen-4-yl-carbonylamino)-3-(2,6-difluorophenyl)propionamide (compound 16);
N-[1(S)-benzoxazol-2-ylcarbonylpropyl]-2(RS)-(2′-chlorobiphen-4-ylcarbonyl-amino)thiazol-2-ylpropionamide (compound 17);
N-[1(S)-(2-ethyl-[1,3,4]-oxadiazol-5-ylcarbonyl)propyl]-2(S)-[2-(2-chlorophenyl)-pyridin-5-ylcarbonylamino]-4(S)phenylpentamide (compound 18);
N-[1(S)-benzoxazol-2-ylcarbonylpropyl]-2(S)-(2,3-diphenylthiophen-5-yl-carbonylamino)-3-(2,6-difluorophenyl)propionamide (compound 19);
N-[1(S)-benzoxazol-2-ylcarbonylpropyl]-2(8)-[2-(2-chlorophenyl)pyridin-5-yl-carbonylamino)-cycloheptylacetamide (compound 20);
N-[1(S)-benzoxazol-2-ylcarbonylpropyl]-2(S)-[2-(2-chlorophenyl)pyridin-1-yl-carbonylamino]-4(S)-phenylpentamide (compound 21);
N-[1(RS)-oxazolo-[4,5-b]-pyridin-2-ylcarbonylpropyl]-2(RS)-2′,3-dichlorobiphen-4-yl-carbonylamino)-cyclohexylacetamide (compound 22);
N-[1(S)-benzoxazol-2-ylcarbonylpropyl]-2(RS)-(4-tert-butylphenylcarbonylamino)-4-ethylhexanoamide (compound 23);
N-[1(S)-benzoxazol-2-ylcarbonylpropyl]-2(5)-2′-Chlorobiphen-4-ylcarbonylamino)-4-ethylhexanoamide (compound 24);
N-[1(S)-benzoxazol-2-ylcarbonylpropyl]-2(S)-(2′-chlorobiphen-4-ylcarbonylamino)-cyclohexylacetamide (compound 25);
N-[1(S)-(2-ethyl-[1,3,4]-oxadiazol-5-ylcarbonyl)butyl]-2(S)-(2′-chlorobiphen-4-yl-carbonylamino)-cyclohexylacetamide (compound 26);
N-[1(S)-benzoxazol-2-ylcarbonylpropyl]-2(S)-(2′-chlorobiphen-4-ylcarbonylamino)-pyrazol-1-ylpropionamide (compound 27);
N-[1(RS)-oxazolo-[4,5-b]-pyridin-2-ylcarbonylpropyl]-2(R)-(2′-chlorobiphen-4-ylcarbonylamino)-4(S)-phenylpentamide (compound 28);
N-[1(S)-benzoxazol-2-ylcarbonylpropyl]-2(RS)-(2′,3-dichlorobiphen-4-ylcarbonyl-amino)-cyclohexylacetamide (compound 29);
N-[1(S)-(2-ethyl-[1,3,4]-oxadiazol-5-ylcarbonylbutyl]-2(S)-(2′,3-dichlorobiphen-4-yl-carbonylamino)-cyclohexylacetamide (compound 30);
N-[1(S)-benzoxazol-2-ylcarbonylpropyl]-2(S)-(5′-carboxy-2′-chlorobiphen-4-yl-carbonylamino)-3-(2,6-difluorophenyl)propionamide (compound 31);
N-[1(S)-benzoxazol-2-ylcarbonylpropyl]-2(S)-(4-morpholin-4-ylphenylcarbonylamino)-4(S)-phenylpentamide (compound 32);
N-[1(S)-oxazolo-[4,5-b]-pyridin-2-ylcarbonylpentyl]-2(RS)-(2′-chlorobiphen-4-yl-carbonylamino)-3-(2,6-difluorophenyl)propionamide (compound 33);
N-[1(S)-benzoxazol-2-ylcarbonylpropyl]-2(S)-(2′-chlorobiphen-4-ylcarbonylamino)-3-(2,4,6-trifluorophenyl)propionamide (compound 34);
N-[1(S)-benzoxazol-2-ylcarbonylpropyl]-2(S)-(1-methyl-3-trifluoro-1H-thieno[2,3-c]-pyrazol-5-ylcarbonylamino)-4S-phenylpentamide (compound 35);
N-[1(S)-benzoxazol-2-ylcarbonylpropyl]-2(S)-(2′-chlorobiphen-4-ylcarbonylamino)-3-thiazol-2-ylpropionamide (compound 36);
N-[1(RS)-benzoxazol-2-ylcarbonylpropyl]-2(S)-[2-(2-chlorophenyl)pyridin-5-yl-carbonylamino)-3-thiazol-2-ylpropionamide (compound 37);
N-[1(S)-benzoxazol-2-ylcarbonylpropyl]-(2S)-[2-(2-chlorophenyl)pyridin-5-yl-carbonylamino)-3-thiazol-2-ylpropionamide (compound 38);
N-[1(S)-benzoxazol-2-ylcarbonylpropyl]-2(S)-(3-vinylphenylcarbonylamino)-4S-phenylpentamide (compound 39);
N-[1(RS)-benzoxazol-2-ylcarbonylpropyl]-2(RS)-(4-phenoxyphenylcarbonylamino)-3-(2,6-difluorophenyl)propionamide (compound 40);
N-[1(S)-benzoxazol-2-ylcarbonylpropyl]-2(S)-(4-acetylamino-3-methylphenylcarbonyl-amino)-4S-phenylpentamide (compound 41);
N-[1(s)-2-phenyl-[1,3,4]-oxadiazol-5-ylcarbonylpropyl]-2(5)-(2′-chlorobiphen-4-yl-carbonylamino)-2-cyclohexylacetamide (compound 42);
N-[1(S)-benzoxazol-2-ylcarbonylpropyl]-2(S)-(2′-chlorobiphen-4-ylcarbonylamino)-3-indol-3-ylpropionamide (compound 43);
N-[1(S)-benzoxazol-2-ylcarbonylpropyl]-2(S)-(2′-chlorobiphen-4-ylcarbonylamino)-3-(N-phenyl-N-methylamino)propionamide (compound 44);
N-[1-(S)-2-phenyl-[1,3,4]-oxadiazol-5-ylcarbonylpropyl]-2(5)-(2′,3-dichlorobiphen-4-ylcarbonylamino)-2-cyclohexylacetamide (compound 45);
N-[1(S)-2-tert-butyl-[1,3,4]-oxadiazol-5-ylcarbonylbutyl]-2(RS)-(2′-chlorobiphen-4-ylcarbonylamino)-3-(2,6-difluorophenyl)propionamide (compound 46);
N-[1(S)-benzoxazol-2-ylcarbonylpropyl]-2(S)-[3,5-di(2-methoxyphenyl)phenylcarbonyl-amino]propionamide (compound 47);
N-[1(S)-benzoxazol-2-ylcarbonylpropyl]-2(RS)-(2′-chlorobiphen-4-ylcarbonylamino)-3-(4-methylindol-3-yl)propionamide (compound 48);
N-[1(S)-benzoxazol-2-ylcarbonylpropyl]-2(S)-(5′-carboxy-2′-chlorobiphen-4-ylcarbonylamino)propionamide (compound 49);
N-[1 (RS)-2-phenyl-[1,3,4]-oxadiazol-5-ylcarbonylpropyl]-2(R)-(2′-chlorobiphen-4-ylcarbonylamino)₄S-phenylpentamide (compound 50);
N-[1(S)-benzoxazol-2-ylcarbonylpropyl]-2(S)-(2′,3-dichlorobiphen-4-ylcarbonylamino)-propionamide (compound 51);
N-[1(S)-benzoxazol-2-ylcarbonylpropyl]-2-(2′-chlorobiphen-4-ylcarbonylamino)-acetamide (compound 52);
N-[1(S)-benzoxazol-2-ylcarbonylpropyl]-2-(5′-carboxy-2′-chlorobiphen-4-yl-carbonylamino)-acetamide (compound 53);
2′-Chlorobiphen-4-ylcarboxylic acid {1-[1(S)-oxazolo-[4,5-b]-pyridin-2-ylcarbonyl)-propylaminocarbonyl]cycloheptyl}amide (compound 54);
2′,3-Dichlorobiphen-4-ylcarboxylic acid {1-[1(S)-xazolo-[4,5-b]-pyridin-2-ylcarbonyl)-propylaminocarbonyl]cycloheptyl}amide (compound 55); and
2′,3-Dichlorobiphen-4-ylcarboxylic acid {1-[1-benzoxazol-2-ylcarbonyl)propylamino-carbonyl]cycloheptyl}amide (compound 56).

17. A pharmaceutical composition comprising a compound of any of the claims 1-16 or a pharmaceutical acceptable salt thereof and a pharmaceutically acceptable excipient.

18. A method of treating a disease in a patient mediated by cathepsins B, K, L, F, and/or which method comprises administering to said patient a pharmaceutical composition comprising a compound of any of the claims 1-16 or a pharmaceutical acceptable salt thereof and a pharmaceutically acceptable excipient.
